(19) United States
(12) Patent Application Publication
(10) Pub. No.: US 2024/0229088 A1
(43) Pub. Date: Jul. 11, 2024

(54) POLYAMINE ANALOG PRODUCING YEASTS

(71) Applicant: CHRYSEA LIMITED, Dublin (IE)

(72) Inventors: Jiufu Qin, Kongens Lyngby (DK); Jens Nielsen, Hellerup (DK)

(21) Appl. No.: 17/768,613

(22) PCT Filed: Oct. 27, 2020
(86) PCT No.: PCT/EP2020/080137

§ 371 Date: Apr. 13, 2022

(30) Foreign Application Priority Data
Oct. 28, 2019 (SE) 1951231-8

(51) Int. Cl. C12P 13/00; C12N 9/00; C12N 9/10; C12N 15/52

(52) U.S. Cl. CPC C12P 13/001; C12N 9/1029; C12N 9/1085; C12N 9/93; C12N 15/52; C12Y 205/01022; C12Y 205/01044; C12Y 602/01012

(57) ABSTRACT

The invention relates to production of polyamine analogs in yeast cells that are capable of producing at least one polyamine. The yeast cells also comprise a 4-coumarate:CoA ligase encoding gene, at least one polyamine N-acyltransferase gene and at least one polyamine synthase encoding gene but lacks a polyamine oxidase encoding gene or comprises a disrupted polyamine oxidase encoding gene. The yeast cells are capable of producing mono- and/or multi-substituted N-acylated polyamines.

### TECHNICAL FIELD

The present invention generally relates to genetically engineered yeasts, and in particular to such yeasts capable of producing polyamine analogs.

### BACKGROUND

Polyamine analogues, which are widely distributed in nature, are being applied to tackle challenges in health and agricultural sectors. For instance, amide bond-containing polyamine analogues, such as N¹-coumaroyl-spermine, N¹-guanyl-1,7-diamine-heptane and N¹,N¹¹-diethyl-norspermine, represent an important class of antivirals, antioxidants, antagonists, and chemotherapeutic agents with potential applications in fighting human diseases, such as cancer and emerging viral threats, e.g., COVID-19. Likewise, widely distributed and amide bond-containing hydroxycinnamic acid amides of the di- and polyamines, such as di-p-coumaroyl-caffeoyl-spermidine, could significantly reduce powdery mildew fungus (Blumeria graminis) infection, demonstrating potential applications in combating fungal pathogens.

However, due to the structural complexity and low abundance in nature, it is hard to obtain polyamine analogs from either traditional synthetic chemistry or extraction from the natural sources. Microbial-based production in fast-growing, genetically tractable species has been pursued as an alternative to the traditional supply chains for natural products and their derivatives. In particular, the baker's yeast Saccharomyces cerevisiae has served as a cell factory for producing many different fuels, chemicals, food ingredients, and pharmaceuticals, in particular for its production of natural products. Indeed, Microbial Cell Factories (2016) 15: 198 discloses cloning of different BAHD acyltransferase coding sequences into a vector containing Arabidopsis thaliana gene At4CL5 for co-expression of a BAHD acyltransferase and At4CL5 in the yeast S. cerevisiae and production of various hydroxycinnamate and benzoate conjugates.

Unfortunately, unlocking the chemical space of polyamine analogues for further pharmacological and insecticidal studies is hindered by several limitations. (i) It is hard to access diverse polyamines, i.e., the precursors for the synthesis or biosynthesis of polyamine analogues from either traditional synthetic chemistry or extraction from natural resources, thereby limiting the diversity of polyamine analogues; (ii) lack of knowledge, e.g., the biosynthetic enzymes, about the biosynthesis of polyamine and polyamine analogues; and (iii) lack of knowledge about the biochemical functions of polyamines, thereby limiting their clinical use.

Consequently, it is desirable to develop new methods for the production of natural polyamines and their analogues, and development of new techniques and value chains for sourcing both natural and unnatural variants of these structures.

### SUMMARY

It is a general objective to provide yeast cells capable of producing polyamine analogs.

This and other objectives are met by the embodiments.

The present invention is defined in the independent claims. Further embodiments of the invention are defined in the dependent claims.

The invention relates to a yeast cell capable of producing at least one polyamine analog. The yeast cell is capable of producing at least one polyamine. The yeast cell also comprises a 4-coumarate:CoA ligase encoding gene, at least one polyamine N-acyltransferase gene and at least one polyamine synthase encoding gene but lacks a polyamine oxidase encoding gene or comprises a disrupted polyamine oxidase encoding gene.

The invention also relates to a method of producing polyamine analogs. The method comprises culturing a yeast cell according to the invention in a culture medium and in culture conditions suitable for production of the polyamine analogs by the yeast cell. The method also comprises collecting the polyamine analogs from the culture medium and/or from the yeast cell.

The present invention provides an efficient means for the production of various polyamine analogs, including mono- and/or multi-substituted N-acylated polyamines. The invention can therefore be used as a cost efficient alternative to prior art methods involving traditional synthetic chemistry or extraction from natural sources to obtain polyamine analogs.

### BRIEF DESCRIPTION OF THE DRAWINGS

The embodiments, together with further objects and advantages thereof, may best be understood by making reference to the following description taken together with the accompanying drawings, in which:

FIGS. 1a to 1f illustrate engineering yeast metabolism for spermidine and higher-polyamine over-synthesis.

FIGS. 2a and 2b illustrate kukosamine production in yeast.

FIGS. 3a to 3f illustrate biosynthesis of complex phenolamides in yeast.

FIGS. 4a to 4c illustrate de novo biosynthesis of complex phenolamides in yeast.

FIGS. 5a and 5b illustrate the engineered pathways for the biosynthesis of complex phenolamides in yeast.

FIG. 6 illustrates in vivo production of fluorine substituted and hydrogenated hydroxycinnamic acid with a p-coumaric acid over-producing stain (QL58) when feeding with fluorine-substituted aromatic amino acid (3-fluoro-L-phenylalanine; 3-F-L-Phe). (6a), fluorine substituted cinnamic acid (3F-CA). (6b), fluorine substituted p-coumaric acid (3-F-pHCA). (6c), fluorine substituted and hydrogenated p-coumaric acid (3-F-DHpHCA). Cell culture supernatant was subject for LC-MS analyses. LC-MS chromatograms were selected for the theoretical m/z values of the respective compounds of interest.

FIG. 7 illustrates in vivo production of fluorine substituted hydroxycinnamic acid-putrescine conjugates with the yeast polyamine platform. (7a), N¹-3-fluorocinnamoyl putrescine. (7b), N¹-3-fluorocoumaroyl putrescine. (7c), N¹-3-fluorohydrocoumaroyl putrescine. (7d), N¹,N⁶-bis(3-fluorocoumaroyl) putrescine. Cell culture supernatant were subject for LC-MS analyses. LC-MS chromatograms were selected for the theoretical m/z values of the respective compounds of interest.

FIG. 8 illustrates in vivo production of fluorine substituted hydroxycinnamic acid-spermidine conjugates with the yeast polyamine platform. (8a), N¹ or N¹⁰-3-fluorocinnamoyl spermidine. (8b), N¹ or N¹⁰-3-fluorocoumaroyl spermidine. (8c), N¹ or N¹⁰-3-fluorohydrocoumaroyl spermidine. (8d), N¹,N¹⁰-bis (3-fluorocoumaroyl) spermidine. (8e), N¹,N⁵,N¹⁰-tri (3-fluorocoumaroyl) spermidine. Cell culture supernatant were subject for LC-MS analyses. LC-MS chromatograms were selected for the theoretical m/z values of the respective compounds of interest.

### DETAILED DESCRIPTION

To enable efficient access to the diversity of polyamine analogs, we engineered yeast metabolism to over-produce a category of complex polyamines, e.g., spermidine, homo-spermidine, thermospermine, and spermine. The versatility of this yeast platform is demonstrated by biosynthesis of diverse polyamine analogs with tailoring pathways. Specially, we systematically refactored yeast central carbon and nitrogen metabolism, methionine salvage pathway, salvage pathways of adenine, polyamine transport machinery, and polyamine degradation pathway, thereby enabling yeasts capable of producing >400 mg/I spermidine in deep-well scale fermentation. Furthermore, by plugging in tailoring pathways and creating synthetic consortium, we demonstrated the de novo biosynthesis of polyamine analogs including tri-substituted N-acylated spermidine phenolamides in yeast.

The present invention now will be described hereinafter with reference to the accompanying drawings and examples, in which embodiments of the invention are shown. This description is not intended to be a detailed catalogue of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined herein, scientific and technical terms used herein will have the meanings that are commonly understood by those of ordinary skill in the art.

Generally, nomenclatures used in connection with techniques of biochemistry, enzymology, molecular and cellular biology, microbiology, genetics and protein and nucleic acid chemistry and hybridization, described herein, are those well-known and commonly used in the art.

Conventional methods and techniques mentioned herein are explained in more detail, for example, in Molecular Cloning, a laboratory manual [second edition] Sambrook et al. Cold Spring Harbor Laboratory, 1989, for example in Sections 1.21 “Extraction And Purification Of Plasmid DNA”, 1.53 “Strategies For Cloning In Plasmid Vectors”, 1.85 “Identification Of Bacterial Colonies That Contain Recombinant Plasmids”, 6 “Gel Electrophoresis Of DNA”, 14 “In vitro Amplification Of DNA By The Polymerase Chain Reaction”, and 17 “Expression Of Cloned Genes In Escherichia coli” thereof.

Enzyme Commission (EC) numbers (also called “classes” herein), referred to throughout this specification, are according to the Nomenclature Committee of the International Union of Biochemistry and Molecular Biology (NC-IUBMB) in its resource “Enzyme Nomenclature” (1992, including Supplements 6-17) available, for example, as “Enzyme nomenclature 1992: recommendations of the Nomenclature Committee of the International Union of Biochemistry and Molecular Biology on the nomenclature and classification of enzymes”, Webb, E. C. (1992), San Diego: Published for the International Union of Biochemistry and Molecular Biology by Academic Press (ISBN 0-12-227164-5). This is a numerical classification scheme based on the chemical reactions catalyzed by each enzyme class.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise. Also as used herein, “and/or” refers to and encompasses any and all possible combinations of one or more of the associated listed items, as well as the lack of combinations when interpreted in the alternative (“or”).

Throughout the description and claims of this specification, the words “comprise” and “contain” and variations of the words, for example “comprising” and “comprises”, mean “including but not limited to” and do not exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

As used herein, the transitional phrase “consisting” essentially of means that the scope of a claim is to be interpreted to encompass the specified materials or steps recited in the claim and those that do not materially affect the basic and novel characteristic(s) of the claimed invention. Thus, the term “consisting” essentially of when used in a claim of this invention is not intended to be interpreted to be equivalent to “comprising.”

To facilitate understanding of the invention, a number of terms are defined below.

As used herein, the term “polyamine” refers to an organic compound having two or more primary amino groups. Examples for polyamines include putrescine (Put), spermidine (Spd), spermine (Spm), thermospermine (Tspm), sym-homospermidine (Hspd), 1,2-diaminopropane, cadaverine, agmatine, sym-norspermidine and norspermine.

As used herein, the terms “polyamine analog”, “polyamine analogue” or “polyamine conjugate” refer to an organic compound formed by reacting a polyamine with at least one molecule to form an amide bond between the polyamine and the at least one molecule. In a particular embodiment, the at least one molecule is at least one carboxyl group comprising molecule thereby allowing a coupling of the carboxylic acid moiety and an amine group of the polyamine. Non-limiting, but preferred, examples of such carboxyl group comprising molecules include aromatic organic acids, such as hydroxycinnamic acids, including α-cyano-4-hydroxycinnamic acid, caffeic acid, chicoric acid, cinnamic acid, chlorogenic acid, diferulic acids, dihydrocaffeic acid, coumaric acid, coumarin, ferulic acid, and sinapinic acid; hydroxycinnamoyltartaric acids, including caftaric acid, coutaric acid and fertaric acid; phenolic acids, including monohydroxybenzoic acids, such as 3-hydroxybenzoic acid, 4-hydroxybenzoic acid, salicylic acid, and p-hydroxybenzoic acid glucoside; dihydroxybenzoic acids, including 2,3-dihydroxybenzoic acid, 2,4-dihydroxybenzoic acid, 2,6-dihydroxybenzoic acid, 3,5-dihydroxybenzoic acid, ethyl protocatechuate, gentisic acid, homogentisic acid, orsellinic acid, and protocatechuic acid; trihydroxybenzoic acids, including bergenin, chebulic acid, ethyl gallate, eudesmic acid, gallic acid, tannic acid, norbergenin, phloroglucinol carboxylic acid, syringic acid and theogallin; vanillin; and ellagic acid. A polyamine analog formed by reacting a polyamine with an aromatic organic acid is typically referred to as a polyamine alkaloid. Other examples of carboxyl group comprising molecules include fatty acids including, but not limited to, saturated fatty acids, such as caprylic acid, capric acid, lauric acid, myristic acid, palmitic acid, stearic acid, arachidic acid, behenic acid, lignoceric acid and cerotic acid; and unsaturated fatty acids, such as myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid and docosahexaenoic acid. A polyamine analog formed by reacting a polyamine with a fatty acid is typically referred to as a polyamine-fatty acid conjugate.

In the art of polyamine nomenclature, generally the number of the atoms within the alkaloids and the lettering of the locants are important. Mainly two numbering systems are used in the literature. Herein, the numbering system as disclosed by Bentz et al 2015 has been used. The system is shortly summarized with the following rules:
I. The numbering of a polyamine skeleton embraces the whole polyamine structure, including the terminal N-atoms;
II. The numbering starts with the N-atom at the end of the shortest carbon chain, e.g., for spermidine with the primary amino group of the aminopropyl subunit;
III. In the case of symmetrical skeletal, e.g., spermine, the numbering starts at the site of the molecules so as to derive the smallest locants for the substituents; and
IV. For N-derivatized polyamines, the locants for N-substituents are prefixed as Nⁿ, with n matching the locant number of the substituted N-atom.

Also as used herein, the terms “nucleotide sequence”, “nucleic acid”, “nucleic acid molecule”, “oligonucleotide” and “polynucleotide” refer to RNA or DNA, including cDNA, a DNA fragment or portion, genomic DNA, synthetic DNA, plasmid DNA, mRNA, and anti-sense RNA, any of which can be single stranded or double stranded, linear or branched, or a hybrid thereof. Nucleic acid molecules and/or nucleotide sequences provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST.25. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made. As used herein the term “recombinant” when used means that a particular nucleic acid (DNA or RNA) is the product of various combinations of cloning, restriction, and/or ligation steps resulting in a construct having a structural coding or non-coding sequence distinguishable from endogenous nucleic acids found in natural systems.

As used herein, the term “gene” refers to a nucleic acid molecule capable of being used to produce mRNA, antisense RNA, miRNA, anti-microRNA antisense oligodeoxyribonucleotide (AMO) and the like. Genes may or may not be capable of being used to produce a functional protein or gene product. Genes can include both coding and non-coding regions, e.g., introns, regulatory elements, promoters, enhancers, termination sequences and/or 5′ and 3′ untranslated regions. A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

A “disrupted gene” as defined herein involves any mutation or modification to a gene resulting in a partial or fully non-functional gene and gene product. Such a mutation or modification includes, but is not limited to, a missense mutation, a nonsense mutation, a deletion, a substitution, an insertion, addition of a targeting sequence and the like. Furthermore, a disruption of a gene can be achieved also, or alternatively, by mutation or modification of control elements controlling the transcription of the gene, such as mutation or modification in a promoter, terminator and/or enhancement elements. In such a case, such a mutation or modification results in partially or fully loss of transcription of the gene, i.e., a lower or reduced transcription as compared to native and non-modified control elements. As a result a reduced, if any, amount of the gene product will be available following transcription and translation. Furthermore, disruption of a gene could also entail adding or removing a localization signal from the gene, resulting in decreased presence of the gene product in its native subcellular compartment.

The objective of gene disruption is to reduce the available amount of the gene product, including fully preventing any production of the gene product, or to express a gene product that lacks or having lower enzymatic activity as compared to the native or wild type gene product.

As used herein the term “deletion” or “knock-out” refers to a gene that is inoperative or knocked out.

The term “attenuated activity” when related to an enzyme refers to a decrease in the activity of the enzyme in its native compartment compared to a control or wildtype state. Manipulations that result in attenuated activity of an enzyme include, but are not limited to, a missense mutation, a nonsense mutation, a deletion, a substitution, an insertion, addition of a targeting sequence, removal of a targeting sequence, or the like. A cell that contains modifications that result in attenuated enzyme activity will have a lower activity of the enzyme compared to a cell that does not contain such modifications. Attenuated activity of an enzyme may be achieved by encoding a nonfunctional gene product, e.g., a polypeptide having essentially no activity, e.g., less than about 10% or even 5% as compared to the activity of the wild type polypeptide.

A codon optimized version of a gene refers to an exogenous gene introduced into a cell and where the codons of the gene have been optimized with regard to the particular cell. Generally, not all tRNAs are expressed equally or at the same level across species. Codon optimization of a gene sequence thereby involves changing codons to match the most prevalent tRNAs, i.e., to change a codon recognized by a low prevalent tRNA with a synonymous codon recognized by a tRNA that is comparatively more prevalent in the given cell. This way the mRNA from the codon optimized gene will be more efficiently translated. The codon and the synonymous codon preferably encode the same amino acid.

As used herein, the terms “peptide”, “polypeptide”, and “protein” are used interchangeably to indicate a polymer of amino acid residues. The terms “peptide”, “polypeptide” and “protein” also includes modifications including, but not limited to, lipid attachment, glycosylation, glycosylation, sulfation, hydroxylation, γ-carboxylation of L-glutamic acid residues and ADP-ribosylation.

As used herein, the term “enzyme” is defined as a protein which catalyzes a chemical or a biochemical reaction in a cell. Usually, according to the present invention, the nucleotide sequence encoding an enzyme is operably linked to a nucleotide sequence (promoter) that causes sufficient expression of the corresponding gene in the cell to confer to the cell the ability to produce spermidine.

As used herein, the term “open reading frame (ORF)” refers to a region of RNA or DNA encoding a polypeptide, a peptide, or a protein.

As used herein, the term “genome” encompasses both the plasmids and chromosomes in a host cell. For instance, encoding nucleic acids of the present disclosure which are introduced into host cells can be portion of the genome whether they are chromosomally integrated or plasmid-localized.

As used herein, the term “promoter” refers to a nucleic acid sequence which has functions to control the transcription of one or more genes, which is located upstream with respect to the direction of transcription of the transcription initiation site of the gene. Suitable promoters in this context include both constitutive and inducible natural promoters as well as engineered promoters, which are well known to the person skilled in the art.

Suitable promoters for use in yeast cells include, but are not limited to, the promoters of PDC, GPD1, TEF1, PGK1 and TDH. Other suitable promoters include the promoters of GAL1, GAL2, GAL10, GAL7, CUP1, HIS3, CYC1, ADH1, PGL, GAPDH, ADC1, URA3, TRP1, LEU2, TPI, AOX1 and ENOI.

As used herein, the term “terminator” refers to a “transcription termination signal” if not otherwise noted.

Terminators are sequences that hinder or stop transcription of a polymerase.

As used herein, “recombinant eukaryotic cells” according to the present disclose is defined as cells which contain additional copies or copy of an endogenous nucleic acid sequence or are transformed or genetically modified with polypeptide or a nucleotide sequence that does not naturally occur in the eukaryotic cells. The wildtype eukaryotic cells are defined as the parental cells of the recombinant eukaryotic cells, as used herein.

As used herein, the terms “increase”, “increased”, “increasing”, “enhance”, “enhanced”, “enhancing”, and “enhancement” (and grammatical variations thereof) indicate an elevation of at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more, or any range therein, as compared to a control.

As used herein, the terms “reduce”, “reduced”, “reduction”, “diminish”, “suppress”, and “decrease” and similar terms mean a decrease of at least about, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more, or any range therein, as compared to a control.

A reduced expression of a gene as used herein involves a genetic modification that reduces the transcription of the gene, reduces the translation of the mRNA transcribed from the gene and/or reduces post-translational processing of the protein translated from the mRNA. Such genetic modification includes insertion(s), deletion(s), replacement(s) or mutation(s) applied to the control 30 sequence, such as a promoter and enhancer, of the gene. For instance, the promoter of the gene could be replaced by a less active or inducible promoter to thereby result in a reduced transcription of the gene. Also a knock-out of the promoter would result in reduced, typically zero, expression of the gene.

As used herein, the term “portion” or “fragment” of a nucleotide sequence of the invention will be understood to mean a nucleotide sequence of reduced length relative to a reference nucleic acid or nucleotide sequence and comprising, consisting essentially of and/or consisting of a nucleotide sequence of contiguous nucleotides identical or almost identical, e.g., 80%, 81%, 82%, 83%, 84%, 85%, 86%, 87%, 88%, 89%, 90%, 91%, 92%, 93%, 94%, 95%, 96%, 98%, 99% identical, to the reference nucleic acid or nucleotide sequence. Such a nucleic acid fragment or portion according to the invention may be, where appropriate, included in a larger polynucleotide of which it is a constituent.

Different nucleic acids or proteins having homology are referred to herein as “homologues”. The term homologue includes homologous sequences from the same and other species and orthologous sequences from the same and other species. “Homology” refers to the level of similarity between two or more nucleic acid and/or amino acid sequences in terms of percent of positional identity, i.e., sequence similarity or identity. Homology also refers to the concept of similar functional properties among different nucleic acids or proteins. Thus, the compositions and methods of the invention further comprise homologues to the nucleotide sequences and polypeptide sequences of this invention. “Orthologous”, as used herein, refers to homologous nucleotide sequences and/or amino acid sequences in different species that arose from a common ancestral gene during speciation. A homologue of a nucleotide sequence of this invention has a substantial sequence identity, e.g., at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, and/or 100%, to said nucleotide sequence.

The term “overexpress”, or “overexpression” as used herein refers to higher levels of activity of a gene, e.g., transcription of the gene; higher levels of translation of mRNA into protein; and/or higher levels of production of a gene product, e.g., polypeptide, than would be in the cell in its native or control, e.g., not transformed with the particular heterologous or recombinant polypeptides being overexpressed, state. A typical example of an overexpressed gene is a gene under transcription control of another promoter as compared to the native promoter of the gene. Also, or alternatively, other changes in the control elements of a gene, such as enhancers, could be used to overexpress the particular gene. Furthermore, modifications that affect, i.e., increase, the translation of the mRNA transcribed from the gene could, alternatively or in addition, be used to achieve an overexpressed gene as used herein. These terms can also refer to an increase in the number of copies of a gene and/or an increase in the amount of mRNA and/or gene product in the cell. It is further possible to achieve overexpression by including genes from different species encoding the same or homologous gene product, such as enzyme.

Overexpression can result in levels that are 25%, 50%, 75%, 100%, 200%, 300%, 400%, 500%, 750%, 1000%, 1500%, 2000% or higher in the cell, or any range therein, as compared to control levels.

As used herein, the terms “exogenous” or “heterologous” when used with respect to a nucleic acid (RNA or DNA), protein or gene refer to a nucleic acid, protein or gene which occurs non-naturally as part of the cell, organism, genome, RNA or DNA sequence, into which it is introduced, including non-naturally occurring multiple copies of a naturally occurring nucleotide sequence. Such an exogenous gene could be a gene from another species or strain, a modified, mutated or evolved version of a gene naturally occurring in the host cell or a chimeric version of a gene naturally occurring in the host cell or fusion genes. In these former cases, the modification, mutation or evolution causes a change in the nucleotide sequence of the gene to thereby obtain a modified, mutated or evolved gene with another nucleotide sequence as compared to the gene naturally occurring in the host cell. Evolved gene refers to genes encoding evolved genes and obtained by genetic modification, such as mutation or exposure to an evolutionary pressure, to derive a new gene with a different nucleotide sequence as compared to the wild type or native gene. A chimeric gene is formed through the combination of portions of one or more coding sequences to produce a new gene. These modifications are distinct from a fusion gene, which merges whole gene sequences into a single reading frame and often retain their original functions.

An “endogenous”, “native” or “wild type” nucleic acid, nucleotide sequence, polypeptide or amino acid sequence refers to a naturally occurring or endogenous nucleic acid, nucleotide sequence, polypeptide or amino acid sequence. Thus, for example, a “wild type mRNA” is an mRNA that is naturally occurring in or endogenous to the organism.

As used herein, the term “modified”, when it is used with respect to an organism, refers to a host organism that has been modified to enable production of at least one polyamine analog, as compared with an otherwise identical host organism that has not been so modified. In principle, such “modification” in accordance with the present disclosure may comprise any physiological, genetic, chemical, or other modification that appropriately alters production of polyamine analogs in a host organism as compared to an otherwise identical organism which is not subject to the modification. In most of the embodiments, however, the modification will comprise a genetic modification. In certain embodiments, as described herein, the modification comprises introducing genes into a host cell. Genetic modifications which boost the activity of a polypeptide include, but are not limited to: introducing one or more copies of a gene encoding the polypeptide (which may distinguish from any gene already present in the host cell encoding a polypeptide having the same activity); altering a gene present in the cell to increase transcription or translation of the gene (e.g., altering, adding additional sequence to, replacement of one or more nucleotides, deleting sequence from, or swapping for example, regulatory, a promoter or other sequence); and altering the sequence (e.g., non-coding or coding) of a gene encoding the polypeptide to boost activity (e.g., by increasing enzyme activity, decrease feedback inhibition, targeting a specific subcellular location, boost mRNA stability, boost protein stability). Genetic modifications that reduce activity of a polypeptide include, but are not limited to: deleting a portion or all of a gene encoding the polypeptide; inserting a nucleic acid sequence which disrupts a gene encoding the polypeptide; changing a gene present in the cell to reduce transcription or translation of the gene or stability of the mRNA or polypeptide encoded by the gene (for example, by adding additional sequence to, altering, deleting sequence from, replacement of one or more nucleotides, or swapping for example, replacement of one or more nucleotides, a promoter, regulatory or other sequence). The term “overproducing” is used herein in reference to the production of a product in a host cell and indicates that the host cell is producing more of product by virtue of the introduction of nucleic acid sequences which encode different polypeptides involved in the host cell's metabolic pathways or as a result of other modifications as compared with the unmodified host cell or wild-type cell.

As used herein the term “vector” is defined as a linear or circular DNA molecule comprising a polynucleotide encoding a polypeptide of the invention, and which is operably linked to additional nucleotides that ensure its expression.

“Introducing” in the context of a yeast cell means contacting a nucleic acid molecule with the cell in such a manner that the nucleic acid molecule gains access to the interior of the cell. Accordingly, polynucleotides and/or nucleic acid molecules can be introduced yeast cells in a single transformation event, in separate transformation events. Thus, the term “transformation” as used herein refers to the introduction of a heterologous nucleic acid into a cell. Transformation of a yeast cell can be stable or transient.

“Transient transformation” in the context of a polynucleotide means that a polynucleotide is introduced into the cell and does not integrate into the genome of the cell.

By “stably introducing” or “stably introduced” in the context of a polynucleotide introduced into a cell, it is intended that the introduced polynucleotide is stably incorporated into the genome of the cell, and thus the cell is stably transformed with the polynucleotide. “Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. Stable transformation as used herein can also refer to a nucleic acid molecule that is maintained extrachromasomally, for example, as a minichromosome.

Transient transformation may be detected by, for example, an enzyme-linked immunosorbent assay (ELISA) or Western blot, which can detect the presence of a peptide or polypeptide encoded by one or more nucleic acid molecules introduced into an organism. Stable transformation of a cell can be detected by, for example, a Southern blot hybridization assay of genomic DNA of the cell with nucleic acid sequences which specifically hybridize with a nucleotide sequence of a nucleic acid molecule introduced into an organism (e.g., a yeast). Stable transformation of a cell can be detected by, for example, a Northern blot hybridization assay of RNA of the cell with nucleic acid sequences which specifically hybridize with a nucleotide sequence of a nucleic acid molecule introduced into a yeast or other organism. Stable transformation of a cell can also be detected by, e.g., a polymerase chain reaction (PCR) or other amplification reaction as are well known in the art, employing specific primer sequences that hybridize with target sequence(s) of a nucleic acid molecule, resulting in amplification of the target sequence(s), which can be detected according to standard methods Transformation can also be detected by direct sequencing and/or hybridization protocols well known in the art.

Embodiments of the present invention also encompass variants of the polypeptides as defined herein. As used herein, a “variant” means a polypeptide in which the amino acid sequence differs from the base sequence from which it is derived in that one or more amino acids within the sequence are substituted for other amino acids. For example, a variant of SEQ ID NO: 1 may have an amino acid sequence at least about 50% identical to SEQ ID NO: 1, for example, at least about 80%, 85%, 90%, 91%, 92%, 93%, 94%, 95%, 96%, 97%, 98%, 99% or about 100% identical. The variants and/or fragments are functional variants/fragments in that the variant sequence has similar or identical functional enzyme activity characteristics to the enzyme having the non-variant amino acid sequence specified herein (and this is the meaning of the term “functional variant” as used throughout this specification).

A “functional variant” or “functional fragment” of any of the presented amino acid sequences, therefore, is any amino acid sequence which remains within the same enzyme category (i.e., has the same EC number) as the non-variant sequences. Methods of determining whether an enzyme falls within a particular category are well known to the skilled person, who can determine the enzyme category without use of inventive skill. Suitable methods may, for example, be obtained from the International Union of Biochemistry and Molecular Biology.

Amino acid substitutions may be regarded as “conservative” where an amino acid is replaced with a different amino acid with broadly similar properties. Non-conservative substitutions are where amino acids are replaced with amino acids of a different type.

By “conservative substitution” is meant the substitution of an amino acid by another amino acid of the same class, in which the classes are defined as follows: Class Amino Acid Examples
Nonpolar: A, V, L, I, P, M, F, W
Uncharged polar: G, S, T, C, Y, N, Q
Acidic: D, E
Basic: K, R, H.

As it is well known to those skilled in the art, altering the primary structure of a polypeptide by a conservative substitution may not significantly alter the activity of that polypeptide because the side-chain of the amino acid which is inserted into the sequence may be able to form similar bonds and contacts as the side chain of the amino acid which has been substituted out. This is so even when the substitution is in a region which is critical in determining the polypeptide's conformation.

In embodiments of the present invention, non-conservative substitutions are possible provided that these do not interrupt the enzyme activities of the polypeptides, as defined elsewhere herein. The substituted versions of the enzymes must retain characteristics such that they remain in the same enzyme class as the non-substituted enzyme, as determined using the NC-IUBMB nomenclature discussed above.

Broadly speaking, fewer non-conservative substitutions than conservative substitutions will be possible without altering the biological activity of the polypeptides. Determination of the effect of any substitution (and, indeed, of any amino acid deletion or insertion) is wholly within the routine capabilities of the skilled person, who can readily determine whether a variant polypeptide retains the enzyme activity according to aspects of the invention. For example, when determining whether a variant of the polypeptide falls within the scope of the invention (i.e., is a “functional variant or fragment” as defined above), the skilled person will determine whether the variant or fragment retains the substrate converting enzyme activity as defined with reference to the NC-IUBMB nomenclature mentioned elsewhere herein. All such variants are within the scope of the invention.

Using the standard genetic code, further nucleic acid sequences encoding the polypeptides may readily be conceived and manufactured by the skilled person, in addition to those disclosed herein. The nucleic acid sequence may be DNA or RNA, and where it is a DNA molecule, it may for example comprise a cDNA or genomic DNA. The nucleic acid may be contained within an expression vector, as described elsewhere herein.

Embodiments of the invention, therefore, encompass variant nucleic acid sequences encoding the polypeptides contemplated by embodiments of the invention. The term “variant” in relation to a nucleic acid sequence means any substitution of, variation of, modification of, replacement of, deletion of, or addition of one or more nucleotide(s) from or to a polynucleotide sequence, providing the resultant polypeptide sequence encoded by the polynucleotide exhibits at least the same or similar enzymatic properties as the polypeptide encoded by the basic sequence. The term includes allelic variants and also includes a polynucleotide (a “probe sequence”) which substantially hybridizes to the polynucleotide sequence of embodiments of the present invention. Such hybridization may occur at or between low and high stringency conditions. In general terms, low stringency conditions can be defined as hybridization in which the washing step takes place in a 0.330-0.825 M NaCl buffer solution at a temperature of about 40-48° C. below the calculated or actual melting temperature (Tm) of the probe sequence (for example, about ambient laboratory temperature to about 55° C.), while high stringency conditions involve a wash in a 0.0165-0.0330 M N buffer solution at a temperature of about 5-10° C. below the calculated or actual Tm of the probe sequence (for example, about 65° C.). The buffer solution may, for example, be a saline-sodium citrate (SSC) buffer (0.15M NaCl and 0.015M tri-sodium citrate), with the low stringency wash taking place in 3×SSC buffer and the high stringency wash taking place in 0.1×SSC buffer. Steps involved in hybridization of nucleic acid sequences have been described for example in Molecular Cloning, a laboratory manual [second edition] Sambrook et al. Cold Spring Harbor Laboratory, 1989, for example in Section 11 “Synthetic Oligonucleotide Probes” thereof.

Preferably, nucleic acid sequence variants have about 80% or more of the nucleotides in common with the nucleic acid sequence of embodiments of the present invention, more preferably at least 85%, or even 90%, 91%, 92%, 93%, 94%, 95%, 96%, 97%, 98% or 99% or greater sequence identity.

Variant nucleic acids of the invention may be codon-optimized for expression in a particular host cell.

As used herein, “sequence identity” refers to sequence similarity between two nucleotide sequences or two peptide or protein sequences. The similarity is determined by sequence alignment to determine the structural and/or functional relationships between the sequences.

Sequence identity between amino acid sequences can be determined by comparing an alignment of the sequences using the Needleman-Wunsch Global Sequence Alignment Tool available from the National Center for Biotechnology Information (NCBI), Bethesda, Md., USA, for example via http://blast.ncbi.nlm.nih.gov/Blast.cgi, using default parameter settings (for protein alignment, Gap costs Existence: 11 Extension: 1). Sequence comparisons and percentage identities mentioned in this specification have been determined using this software. When comparing the level of sequence identity to, for example, SEQ ID NO: 1, this, preferably should be done relative to the whole length of SEQ ID NO: 1 (i.e., a global alignment method is used), to avoid short regions of high identity overlap resulting in a high overall assessment of identity. For example, a short polypeptide fragment having, for example, five amino acids might have a 100% identical sequence to a five amino acid region within the whole of SEQ ID NO: 1, but this does not provide a 100% amino acid identity unless the fragment forms part of a longer sequence which also has identical amino acids at other positions equivalent to positions in SEQ ID NO: 1. When an equivalent position in the compared sequences is occupied by the same amino acid, then the molecules are identical at that position. Scoring an alignment as a percentage of identity is a function of the number of identical amino acids at positions shared by the compared sequences. When comparing sequences, optimal alignments may require gaps to be introduced into one or more of the sequences, to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties. As mentioned above, the percentage sequence identity may be determined using the Needleman-Wunsch Global Sequence Alignment tool, using default parameter settings. The Needleman-Wunsch algorithm was published in J. Mol. Biol. (1970) vol. 48: 443-453.

An aspect of the invention relates to a yeast cell capable of producing at least one polyamine analog. The yeast cell is capable of producing at least one polyamine. The yeast cell comprises at least one Coenzyme A (CoA) ligase encoding gene, preferably a 4-coumarate:CoA ligase encoding gene, at least one polyamine N-acyltransferase gene and at least one polyamine synthase encoding gene but lacks a polyamine oxidase encoding gene or comprises a disrupted polyamine oxidase encoding gene.

The yeast cell of the invention comprises a gene encoding a 4-coumarate:CoA ligase (EC 6.2.1.12) that is capable of converting a carboxyl group comprising molecule into a CoA ester. The corresponding CoA ester is then a substrate of the polyamine N-acyltransferase together with the at least one polyamine produced by the yeast cell to thereby obtain the at least one polyamine analog by acetylating the least one polyamine analog and forming an amide bound between the at least one polyamine and the CoA ester.

In an embodiment, the yeast cell is engineered for overexpression of a 4-coumarate:CoA ligase.

The overexpression of the 4-coumarate:CoA ligase is, in an embodiment, achieved by putting the 4-coumarate:CoA ligase encoding gene under transcriptional control of a promoter that is highly active in the yeast cell. Suitable promoters for use in yeast cells include, but are not limited to, the promoters of PDC, GPD, GPD1, TEF1, PGK1, TDH and TDH3. Other suitable promoters include the promoters of GAL1, GAL2, GAL10, GAL7, CUP1, HIS3, CYC1, ADH1, PGL, GAPDH, ADC1, URA3, TRP1, LEU2, TPI, AOX1 and ENOI.

The yeast cell can comprise one or multiple, i.e., at least two, copies of the 4-coumarate:CoA ligase encoding gene to thereby increase the copy number of the mRNA for the 4-coumarate:CoA ligase and thereby the amount of 4-coumarate:CoA ligase produced by the yeast cell. In such a case, the multiple copies of the 4-coumarate:CoA ligase encoding gene could be under transcription control of one promoter, or each 4-coumarate:CoA ligase encoding gene could be under transcription control of a respective promoter. In the latter case, a same type of promoter could be used to control transcription of the respective 4-coumarate:CoA ligase encoding genes or different types of promoters could be used.

In an embodiment, the 4-coumarate:CoA ligase (4CL) encoding gene is selected from the group consisting of Arabidopsis thaliana 4-coumarate:CoA ligase 1 (At4CL1), At4CL2, At4CL3, At4CL4, At4CL5 and a nucleotide sequence encoding a 4-coumarate:CoA ligase having at least 80% sequence identity with any of 4-coumarate:CoA ligase At4CL1, At4CL2, At4CL3, At4CL4, or At4CL5. In an embodiment, the nucleotide sequence encodes a 4-coumarate:CoA ligase having at least 85%, or even 90%, 91%, 92%, 93%, 94%, 95%, 96%, 97%, 98% or 99% sequence identity with any of Arabidopsis thaliana 4CL1 (SEQ ID NO: 1), 4CL2, 4CL3, 4CL4 or 4CL5. In an embodiment, this 4-coumarate:CoA ligase having at least 80% sequence identity is capable of catalyzing conversion of a carboxyl group comprising molecule into a CoA ester, preferably capable of catalyzing conversion of 4-coumarate into 4-coumaroyl-CoA. The enzymatic efficacy of the 4-coumarate:CoA ligase having at least 80% sequence identity may be lower, substantially equal to or higher than the corresponding enzymatic efficacy of the relevant 4-coumarate:CoA ligase, preferably at least substantially equal to or higher enzymatic efficacy.

In a particular embodiment, the 4-coumarate:CoA ligase encoding gene is At4CL1. The amino acid sequence for At4CL1 is shown in SEQ ID NO: 1 and the nucleotide sequence for At4CL1 is shown in SEQ ID NO: 2.

In an embodiment, the yeast cell is engineered for overexpression of the at least one polyamine N-acyltransferase.

The overexpression of the at least one polyamine N-acyltransferase is, in an embodiment, achieved by putting at least one polyamine N-acyltransferase encoding gene under transcriptional control of a promoter that is highly active in the yeast cell. Suitable promoters for use in yeast cells include, but are not limited to, the promoters of PDC, GPD, GPD1, TEF1, PGK1, TDH and TDH3. Other suitable promoters include the promoters of GAL1, GAL2, GAL10, GAL7, CUP1, HIS3, CYC1, ADH1, PGL, GAPDH, ADC1, URA3, TRP1, LEU2, TPI, AOX1 and ENOI.

The yeast cell can comprise one or multiple copies of the polyamine N-acyltransferase encoding gene to thereby increase the copy number of the mRNA for the polyamine N-acyltransferase and thereby the amount of polyamine N-acyltransferase produced by the yeast cell. In such a case, the multiple copies of the polyamine N-acyltransferase encoding gene could be under transcription control of one promoter, or each polyamine N-acyltransferase encoding gene could be under transcription control of a respective promoter. In the latter case, a same type of promoter could be used to control transcription of the respective polyamine N-acyltransferase encoding genes or different types of promoters could be used.

In an embodiment, the yeast cell comprises the at least one polyamine N-acyltransferase encoding gene selected from the group consisting of a spermidine hydroxycinnamoyl transferase (EC 2.3.1.M34) encoding gene, a spermidine coumaroyl-CoA acyltransferase (EC 2.3.1.249) encoding gene and a putrescine hydroxycinnamoyl transferase (EC 2.3.1.138) encoding gene.

In a particular embodiment, the spermidine hydroxycinnamoyl transferase (SHT) encoding gene is selected from the group consisting of Arabidopsis thaliana spermidine hydroxycinnamoyl transferase (AtSHT), Nicotiana attenuata DH29 (NaDH29) and a nucleotide sequence encoding a spermidine hydroxycinnamoyl transferase having at least 80% sequence identity with spermidine hydroxycinnamoyl transferase AtSHT (SEQ ID NO: 3) or spermidine hydroxycinnamoyl transferase NaDH29 (SEQ ID NO: 5). In an embodiment, the nucleotide sequence encodes a spermidine hydroxycinnamoyl transferase having at least 85%, or even 90%, 91%, 92%, 93%, 94%, 95%, 96%, 97%, 98% or 99% sequence identity with any of Arabidopsis thaliana SHT or Nicotiana attenuata DH29. In an embodiment, this spermidine hydroxycinnamoyl transferase having at least 80% sequence identity is capable of catalyzing conversion of spermidine and a CoA ester into a polyamine analog, preferably capable of catalyzing conversion of spermidine and coumaroyl-CoA, feruloyl-CoA, caffeoyl-CoA, cinnamoyl-CoA or sinapoyl-CoA into N¹-(coumaroyl, feruloyl, caffeoyl, cinnamoyl or sinapoyl) spermidine, N¹⁰-(coumaroyl, feruloyl, caffeoyl, cinnamoyl or sinapoyl) spermidine, N¹,N¹⁰-bis(coumaroyl, feruloyl, caffeoyl, cinnamoyl or sinapoyl) spermidine and/or N¹,N⁵,N¹⁰-tri(coumaroyl, feruloyl, caffeoyl, cinnamoyl or sinapoyl) spermidine. The enzymatic efficacy of the spermidine hydroxycinnamoyl transferase having at least 80% sequence identity may be lower, substantially equal to or higher than the corresponding enzymatic efficacy of the relevant spermidine hydroxycinnamoyl transferase, preferably at least substantially equal to or higher enzymatic efficacy.

The SHT encoding gene is catalyzing the production of polyamine analogs in the yeast cell in terms of polyamine alkaloids and in particular mono-substituted, di-substituted (also referred to as bis-substituted) and/or tri-substituted N-acylated polyamines, preferably spermidine. In a particular embodiment, expression of such an SHT encoding gene together with the 4CL encoding gene enables the yeast cell to produce symmetrical tri-substituted N-acylated polyamines, preferably spermidine, in the case of AtSHT and symmetrical single-substituted N-acylated polyamines, preferably spermidine, in the case of NaDH29.

The amino acid sequence for AtSHT is shown in SEQ ID NO: 3 and the nucleotide sequence for AtSHT is shown in SEQ ID NO: 4. The corresponding amino acid sequence for NaDH29 is shown in SEQ ID NO: 5 and the nucleotide sequence for NaDH29 is shown in SEQ ID NO: 6.

In an embodiment, the spermidine coumaroyl-CoA acyltransferase (SCT) encoding gene is selected from the group consisting of Arabidopsis thaliana spermidine coumaroyl-CoA acyltransferase (AtSCT) and a nucleotide sequence encoding a spermidine coumaroyl-CoA acyltransferase having at least 80% sequence identity with spermidine coumaroyl-CoA acyltransferase AtSCT. In an embodiment, the nucleotide sequence encodes a spermidine coumaroyl-CoA acyltransferase having at least 85%, or even 90%, 91%, 92%, 93%, 94%, 95%, 96%, 97%, 98% or 99% sequence identity with Arabidopsis thaliana SCT (SEQ ID NO: 7). In an embodiment, this spermidine coumaroyl-CoA acyltransferase having at least 80% sequence identity is capable of catalyzing conversion of spermidine and a CoA ester into a polyamine analog, preferably capable of catalyzing conversion of spermidine and coumaroyl-CoA, feruloyl-CoA, caffeoyl-CoA, cinnamoyl-CoA or sinapoyl-CoA into N¹,N¹⁰-bis(coumaroyl, feruloyl, caffeoyl, cinnamoyl or sinapoyl) spermidine. The enzymatic efficacy of the spermidine coumaroyl-CoA acyltransferase having at least 80% sequence identity may be lower, substantially equal to or higher than the corresponding enzymatic efficacy of AtSCT, preferably at least substantially equal to or higher enzymatic efficacy.

The SCT encoding gene is catalyzing the production of polyamine analogs in the yeast cell in terms of polyamine alkaloids and in particular di-substituted N-acylated polyamines, preferably spermidine. In a particular embodiment, expression of such an SCT encoding gene together with the 4CL encoding gene enables the yeast cell to produce symmetrical di-substituted N-acylated polyamines, preferably spermidine, in the case of AtSCT.

The amino acid sequence for AtSCT is shown in SEQ ID NO: 7 and the nucleotide sequence for AtSCT is shown in SEQ ID NO: 8.

In an embodiment, the putrescine hydroxycinnamoyl transferase encoding gene is selected from the group consisting of Nicotiana attenuata AT1 (NaAT1) and a nucleotide sequence encoding a putrescine hydroxycinnamoyl transferase having at least 80% sequence identity with putrescine hydroxycinnamoyl transferase NaAT1. In an embodiment, the nucleotide sequence encodes a putrescine hydroxycinnamoyl transferase having at least 85%, or even 90%, 91%, 92%, 93%, 94%, 95%, 96%, 97%, 98% or 99% sequence identity with Nicotiana attenuata AT1 (SEQ ID NO: 9). In an embodiment, this putrescine hydroxycinnamoyl transferase having at least 80% sequence identity is capable of catalyzing conversion of putrescine and a CoA ester into a polyamine analog, preferably capable of catalyzing conversion of putrescine and coumaroyl-CoA, feruloyl-CoA, caffeoyl-CoA, cinnamoyl-CoA or sinapoyl-CoA into N¹-(coumaroyl, feruloyl, caffeoyl, cinnamoyl or sinapoyl) putrescine, N⁶-bis(coumaroyl, feruloyl, caffeoyl, cinnamoyl or sinapoyl) putrescine and/or N¹,N⁶-bis(coumaroyl, feruloyl, caffeoyl, cinnamoyl or sinapoyl) putrescine. The enzymatic efficacy of the putrescine hydroxycinnamoyl transferase having at least 80% sequence identity may be lower, substantially equal to or higher than the corresponding enzymatic efficacy of NaAT1, preferably at least substantially equal to or higher enzymatic efficacy.

The putrescine hydroxycinnamoyl transferase encoding gene is catalyzing the production of polyamine analogs in the yeast cell in terms of polyamine alkaloids and in particular di-substituted N-acylated polyamines, preferably putresceine. In a particular embodiment, expression of such a putrescine hydroxycinnamoyl transferase encoding gene together with the 4CL encoding gene enables the yeast cell to produce symmetrical di-substituted N-acylated polyamines, preferably putrescine, in the case of NaAT1.

The amino acid sequence for NaAT1 is shown in SEQ ID NO: 9 and the nucleotide sequence for NaAT1 is shown in SEQ ID NO: 10.

In an embodiment, the yeast cell comprises more than one type of polyamine N-acyltransferase encoding gene. Hence, the embodiments encompass a yeast cell comprising a 4CL encoding gene, such as At4CL1, an SHT encoding gene, such as AtSHT and/or NaDH29, and an SCT encoding gene, such as AtSCT, a yeast cell comprising a 4CL encoding gene, such as At4CL1, an SHT encoding gene, such as AtSHT and/or NaDH29, and a putrescine hydroxycinnamoyl transferase encoding gene, such as NaAT1; a yeast cell comprising a 4CL encoding gene, such as At4CL1, an SCT encoding gene, such as AtSCT, and a putrescine hydroxycinnamoyl transferase encoding gene, such as NaAT1; and a yeast cell comprising a 4CL encoding gene, such as At4CL1, an SHT encoding gene, such as AtSHT and/or NaDH29, an SCT encoding gene, such as AtSCT, and a putrescine hydroxycinnamoyl transferase encoding gene, such as NaAT1. For instance, a yeast cell that comprises a 4CL encoding gene, such as At4CL1, an SCT encoding gene, such as AtSCT, and an SHT encoding gene, such as AtSHT, is capable of producing asymmetric tri-substituted N-acylated polyamines, preferably spermidine.

In an embodiment, the carboxyl group comprising molecule, such as aromatic organic acid(s), fatty acid(s), halogenated aromatic organic acid(s), halogenated fatty acid(s) or a combination thereof, is added to the culture medium, in which the yeast cell is cultured. Hence, in this embodiment the yeast cell is fed the carboxyl group comprising molecule that is converted into a CoA ester by the 4-coumarate:CoA ligase expressed by the yeast cell. The yeast cell may then be fed a single carboxyl group comprising molecule or a mixture of different carboxyl group comprising molecules. For instance, a yeast cell comprising a 4CL encoding gene, such as At4CL1, and a SHT encoding gene, such as AtSHT, is capable of producing asymmetric tri-substituted N-acylated polyamines, such as spermidine, when fed a mixture of aromatic organic acids. Correspondingly, a yeast cell comprising a 4CL encoding gene, such as At4CL1, and a SCT encoding gene, such as AtSCT, is capable of producing asymmetric di-substituted N-acylated polyamines, such as spermidine, when fed a mixture of aromatic organic acids. A yeast cell comprising a 4CL encoding gene, such as At4CL1, and an SHT encoding gene, such as NaDH29, is capable of producing symmetric mono-substituted N-acylated polyamines, such as spermidine, when fed a mixture of aromatic organic acids.

Instead of, or as a complement to, feeding the yeast cell with the carboxyl group comprising molecule(s), such as aromatic organic acid(s) and/or fatty acid(s), and/or halogenated versions thereof, the yeast cell can be engineered to produce or overproduce the carboxyl group comprising molecule(s). Hence, in a particular embodiment, the yeast cell is capable of producing at least one organic acid selected from the group consisting of an aromatic organic acid, a halogenated aromatic organic acid, a fatty acid, a halogenated fatty acid and a combination thereof. Yeast cells engineered for production of such aromatic organic acids and/or fatty acids are disclosed in Yu et al. 2018; Zhou et al. 2016; Liu et al. 2019; and Rodriguez et al. 2015, the teachings of which with regard to a yeast cell is capable of producing at least one organic acid selected from the group consisting of an aromatic organic acid, a halogenated aromatic organic acid, a fatty acid, a halogenated fatty acid and a combination thereof is hereby incorporated by reference.

However, overproducing such carboxyl group comprising molecules in the yeast cell of the invention may cause flux unbalances between polyamine metabolism and the metabolism of the carboxyl group comprising molecule(s), such as in terms of aromatic amino acid (AAA) metabolism. Another source for the carboxyl group comprising molecules instead of, or as a complement to, feeding the yeast cells by adding the carboxyl group comprising molecule(s) to the culture medium is to co-culture the yeast cell of the invention with a microbial cell, preferably a yeast cell, capable of producing and secreting the carboxyl group comprising molecule(s). Non-limiting, but illustrative, examples of such microbial cells that can be co-cultured with the yeast cell of the invention are disclosed in Yu et al. 2018; Zhou et al. 2016; Liu et al. 2019; and Rodriguez et al. 2015.

As used herein, a halogenated aromatic organic acid includes a halogen-substituted aromatic organic acid and a halogenated fatty acid includes a halogen-substituted fatty acid. Illustrative examples of such halogen-substituted aromatic organic acid and fatty acids include fluorine-substituted, chlorine-substituted, bromine-substituted and iodine-substituted aromatic organic acid and fatty acids, preferably fluorine-substituted aromatic organic acid and fatty acids.

In an embodiment, the at least one polyamine is selected from the group consisting of spermine, thermospermine, sym-homospermidine, 1,3-diaminopropane, putrescine, cadaverine, agmatine, spermidine, sym-norspermidine, norspermine and a combination thereof.

The yeast cell of the invention lacks a polyamine oxidase (EC 1.5.3.17) encoding gene or comprises a disrupted polyamine oxidase encoding gene. The yeast cell also comprises at least one polyamine synthase encoding gene.

The at least one polyamine synthase expressed by the yeast cell catalyzes the production of at least one polyamine in the yeast cell. Polyamine oxidase is an enzyme that catalyzes the conversion of spermine back to spermidine. Hence, the yeast cell lacks any polyamine oxidase encoding gene or comprises a disrupted polyamine oxidase encoding gene. This means that the yeast cell preferably lacks any polyamine oxidase or, if such a polyamine oxidase is expressed in the yeast cell, the polyamine oxidase is preferably enzymatically inactive or at least has significantly lower enzymatic efficiency as compared to the native polyamine oxidase.

In an embodiment, the yeast cell is engineered for overexpression of the at least one polyamine synthase.

The overexpression of the at least one polyamine synthase is, in an embodiment, achieved by putting the at least one at least one polyamine synthase encoding gene under transcriptional control of a promoter that is highly active in the yeast cell. Suitable promoters for use in yeast cells include, but are not limited to, the promoters of PDC, GPD, GPD1, TEF1, PGK1, TDH and TDH3. Other suitable promoters include the promoters of GAL1, GAL2, GAL10, GAL7, CUP1, HIS3, CYC1, ADH1, PGL, GAPDH, ADC1, URA3, TRP1, LEU2, TPI, AOX1 and ENOI.

The yeast cell can comprise one or multiple copies of the polyamine synthase encoding gene to thereby increase the copy number of the mRNA for the polyamine synthase and thereby the amount of polyamine synthase produced by the yeast cell. In such a case, the multiple copies of the polyamine synthase encoding gene could be under transcription control of one promoter, or each polyamine synthase encoding gene could be under transcription control of a respective promoter. In the latter case, a same type of promoter could be used to control transcription of the respective polyamine synthase encoding genes or different types of promoters could be used.

In an embodiment, the polyamine synthase encoding gene is selected from the group consisting of a spermine synthase (EC 2.5.1.22) encoding gene, a thermospermine synthase (EC 2.5.1.79) encoding gene and a homospermidine synthase (EC 2.5.1.44 or EC 2.5.1.45) encoding gene.

In an embodiment, the spermine synthase encoding gene is selected from the group consisting of Saccharomyces cerevisiae spermine synthase, preferably ScSPE4, Arabidopsis thaliana spermine synthase (AtSPMS) and a nucleotide sequence encoding a spermine synthase having at least 80% sequence identity with spermine synthase ScSPE4 or spermine synthase AtSPMS. In an embodiment, the nucleotide sequence encodes a spermine synthase having at least 85%, or even 90%, 91%, 92%, 93%, 94%, 95%, 96%, 97%, 98% or 99% sequence identity with ScSPE4 or AtSPMS. In an embodiment, this spermine synthase having at least 80% sequence identity is capable of catalyzing conversion of spermidine into spermine. The enzymatic efficacy of the spermine synthase having at least 80% sequence identity may be lower, substantially equal to or higher than the corresponding enzymatic efficacy of ScSPE4 or AtSPES, preferably at least substantially equal to or higher enzymatic efficacy.

The amino acid sequence for ScSPE4 is shown in SEQ ID NO: 11 and the nucleotide sequence for ScSPE4 is shown in SEQ ID NO: 12. The corresponding amino acid sequence for AtSPMS is shown in SEQ ID NO: 13 and the nucleotide sequence of AtSPMS is shown in SEQ ID NO: 14.

In an embodiment, the thermospermine synthase encoding gene is selected from the group consisting of Arabidopsis thaliana thermospermine synthase, preferably AtACL5, and a nucleotide sequence encoding a thermospermine synthase having at least 80% sequence identity with thermospermine synthase AtACL5. In an embodiment, the nucleotide sequence encodes a thermospermine synthase having at least 85%, or even 90%, 91%, 92%, 93%, 94%, 95%, 96%, 97%, 98% or 99% sequence identity with AtACL5. In an embodiment, this thermospermine synthase having at least 80% sequence identity is capable of catalyzing conversion of spermidine into thermospermine. The enzymatic efficacy of the thermospermine synthase having at least 80% sequence identity may be lower, substantially equal to or higher than the corresponding enzymatic efficacy of AtACL5, preferably at least substantially equal to or higher enzymatic efficacy.

The amino acid sequence for AtACL5 is shown in SEQ ID NO: 15 and the nucleotide sequence for AtACL5 is shown in SEQ ID NO: 16.

In an embodiment, the homospermidine synthase (HSS) encoding gene is selected from the group consisting of Senecio vernalis homospermidine synthase (SvHSS), Blastochloris viridis homospermidine synthase (BvHSS) and a nucleotide sequence encoding a homospermidine synthase having at least 80% sequence identity with homospermidine synthase SvHSS or homospermidine synthase BvHSS. In an embodiment, the nucleotide sequence encodes a homospermidine synthase having at least 85%, or even 90%, 91%, 92%, 93%, 94%, 95%, 96%, 97%, 98% or 99% sequence identity with SvHSS or BvHSS. In an embodiment, this homospermidine synthase having at least 80% sequence identity is capable of catalyzing conversion of putrescine into sym-homospermidine, or putrescine or spermidine into sym-homospermidine. The enzymatic efficacy of the homospermidine synthase having at least 80% sequence identity may be lower, substantially equal to or higher than the corresponding enzymatic efficacy of SvHSS or BvHSS, preferably at least substantially equal to or higher enzymatic efficacy.

The amino acid sequence for SvHSS is shown in SEQ ID NO: 17 and the nucleotide sequence for SvHSS is shown in SEQ ID NO: 18. The corresponding amino acid sequence for BvHSS is shown in SEQ ID NO: 19 and the nucleotide sequence for BvHSS is shown in SEQ ID NO: 20.

In an embodiment, the yeast cell selected from a group consisting of Saccharomyces, Kluyveromyces, Zygosaccharomyces, Candida, Hanseniaspora, Pichia, Hansenula, Schizosaccharomyces, Trigonopsis, Brettanomyces, Debaromyces, Nadsonia, Lipomyces, Cryptococcus, Aureobasidium, Trichosporon, Rhodotorula, Yarrowia, Rhodosporidium, Phaffia, Schwanniomyces, Aspergillus and Ashbya. In a particular embodiment, the yeast cell is selected from the group consisting of Saccharomyces cerevisiae, Saccharomyces boulardii, Zygosaccharomyces bailii, Kluyveromyces lactis, Rhodosporidium toruloides, Yarrowia lipolytica, Schizosaccharomyces pombe, Pichia pastoris, Hansenula anomala, Candida sphaerica, or Schizosaccharomyces malidevorans. Saccharomyces cerevisiae is a preferred yeast species.

In an embodiment, the yeast cell is a Saccharomyces cerevisiae cell and the polyamine oxidase is FMS1. Hence, in an embodiment, the S. cerevisiae cell lacks FMS1 or comprises a disrupted FMS1.

Another aspect of the invention relates to a yeast cell capable of producing at least one polyamine analog. The yeast cell is capable of producing at least one polyamine. The yeast cell comprises a 4-coumarate:CoA ligase encoding gene and at least one polyamine N-acyltransferase gene.

Various embodiments of the yeast cell as described in the foregoings can also be applied to this aspect of the invention.

A further aspect of the invention relates to a method of producing polyamine analogs. The method comprises culturing a yeast cell according to the invention, in a culture medium and in culture conditions suitable for production of the polyamine analogs by the yeast cell. The method also comprises collecting the polyamine analogs from the culture medium and/or from the yeast cell.

In an embodiment, culturing the yeast cell comprises culturing the yeast cell in the culture medium comprising at least one organic acid selected from the group consisting of an aromatic organic acid, a fatty acid and a combination thereof. Hence, in this embodiment, the carboxyl group comprising molecule in the form of at least one organic acid is comprised in the culture medium. Hence, the yeast cells is fed the at least one organic acid.

In a particular embodiment, the method comprises adding the at least one organic acid to the culture medium. Thus, the at least one organic acid is available for the yeast cell in the culture medium by adding the at least one organic acid to the culture medium. In this particular embodiment, a single organic acid is added to the culture medium or multiple different organic acids are added to the culture medium.

In another particular embodiment, culturing the yeast cell comprises co-culturing the yeast cell in the culture medium with a microorganism, preferably a yeast cell, capable of producing the at least one organic acid and releasing the at least one organic acid into the culture medium.

The two described particular embodiments can be combined, i.e., adding at least one organic acid to the culture medium, in which the yeast cell of the invention is co-cultured with at least one microorganism, preferably yeast cell, capable of producing at least one organic acid. The at least one organic acid added to the culture medium could be the same or different from the at least one organic acid produced by the at least one microorganism.

The culture medium in this aspect of the invention can be any culture medium, in which the yeast cell can be cultured to produce polyamine analogs. The culturing can be in the form of, for instance, batch, fed-batch or perfusion culturing or fermentation, bioreactor fermentation, etc.

### EXAMPLES

### Example 1: Improvement of Spermidine Production by Systematic Rewiring of Native Metabolism in Yeast

In this Example 1, we systematically refactored metabolism in a yeast strain, including central carbon and nitrogen metabolism, methionine salvage pathway, salvage pathways of adenine, polyamine transport machinery, and polyamine consumption/degradation pathway. In addition, we also introduced extra potential positive genetic targets. This yeast strain built with a new modular genetic design. Specially, the de novo Spd biosynthetic pathway split into multiple genetic modules that contain the coding sequences for numerous biosynthetic enzymes to divert greater carbon flux from sugar carbon source to Spd.

A precursor overproduction module (I) designed to increase accumulation of L-ornithine (Orn) including the overexpression of eight proteins: NADPH-dependent glutamate dehydrogenase from Saccharomyces cerevisiae (GDH1) [SEQ ID NO: 21], mitochondrial aspartate and glutamate carrier protein from S. cerevisiae (AGC1) [SEQ ID NO: 22], mitochondrial L-ornithine carrier protein from S. cerevisiae (ORT1) [SEQ ID NO: 23], glutamate N-acetyltransferase from Escherichia coli (EcargA) [SEQ ID NO: 24], acetylglutamate kinase from E. coli (EcargB) [SEQ ID NO: 25], N-acetyl-gamma-glutamyl-phosphate reductase from Corynebacterium glutamicum (CgargC) [SEQ ID NO: 26], acetylornithine aminotransferase from C. glutamicum (CgargD) [SEQ ID NO: 27], and ornithine acetyltransferase from C. glutamicum (CgargJ) [SEQ ID NO: 28]. Moreover, attenuation or removal of two proteins was also included in this module (I): attenuation of yeast native ornithine carbamoyltransferase (ARG3) [SEQ ID NO: 29] by swapping its native promoter P_{ARG3} with weaker promoter P_{KEX2}, and removing the activity of L-ornithine transaminase (CAR2) [SEQ ID NO: 30] by knockout of CAR2.

A putrescine (Put) module (II) designed to overproduce Put from L-ornithine included two genetic modifications; the overexpression of ornithine decarboxylase from S. cerevisiae (SPE1) [SEQ ID NO: 31] and the deletion of native ornithine decarboxylase antizyme (OAZ1) [SEQ ID NO: 32].

A spermidine biosynthesis module (III) was designed for overproduction of spermidine (Spd) from putrescine and was characterized by overexpression of two proteins from S. cerevisiae: adenosylmethionine decarboxylase (AdoMetDC; SPE2) [SEQ ID NO: 33] and spermidine synthase (SpdSyn; SPE3) [SEQ ID NO: 34]. This module also included the deletion of two native proteins to avoid spermidine consumption or degradation: deletion of SPE4 [SEQ ID NO: 12] encoding spermine synthase and FMS1 [SEQ ID NO: 35] encoding a non-specific polyamine oxidase.

An S-adenosyl-L-methionine (AdoMet) module (IV) was designed to increase the accessibility of co-factor AdoMet. These modifications included overexpression of numerus proteins: 5′-methylthioadenosine phosphorylase (MEU1) [SEQ ID NO: 36] from S. cerevisiae, branched-chain amino acid aminotransferase (BAT2) [SEQ ID NO: 37] from S. cerevisiae, adenine phosphoribosyltransferase (APT1) [SEQ ID NO: 38] from S. cerevisiae, ribose-phosphate pyrophosphokinases (PRS5) [SEQ ID NO: 39] from S. cerevisiae, and S-adenosylmethionine synthetase from Leishmania infantum (LiMAT) [SEQ ID NO: 40]. This module also included deletion of adenine deaminase activity (AAH1) [SEQ ID NO: 41].

A polyamine efflux module (V) was designed to relieve the cytotoxicity to the cell or the inhibition to the polyamine biosynthesis. This module included the overexpression of yeast native polyamine transporter encoded byTPO5 [SEQ ID NO: 42].

Finally yet importantly, an extra spermidine biosynthesis module (VI) was designed for overproduction of spermidine from putrescine and AdoMet. This module included overexpression of an AdoMetDC-SpdSyn fusion protein encoded by SPE2-SPE3 [SEQ ID NO: 43].

The overexpression of genes in this Example 1 was obtained by chromosomal integrations to the regions from which we expected no growth defect and active expression as integration loci via the CRISPR/cas9 system or the traditional genetic makers based methods. The implementation of CRISPR/cas9 based genome editing followed the protocol developed by Mans et al. 2015. In particular, S. cerevisiae strain CEN.PK113-11C harboring plasmid pL-CAS9-HIS with HIS3 marker, enabling the constitutive expression of Cas9, was the start strain for all genetic manipulations. To enable the efficient genome editing in the selected loci, multiple guide RNA (gRNA) plasmids were constructed.

Genetic modules, including various combinations of genetic parts, i.e., promoters, terminators, ORFs and homologous arms constructed as integration cassettes according to overlapping extension PCR (OE-PCR) procedure. The following gene and promoter combinations where used in this Example 1: TP11p-ORT1-pYX212t; tHXT7p-AGC1-CYC1t; TEF1p-GDH1-DIT1t; PGK1p-SPE3-pYX212t; TEF1p-SPE1-PRM9t; TDH3p-SPE2-DIT1t; TDH3p-CgargJ-TDH2t; PGK1p-EcargB-ADH1t; TEF1p-CgargC-FBA1t; tHXT7p-CgargD-TP11t; TP11p-EcargA-CYC1t; TP11p-MEU1p-FBA1t; PGK1p-BAT2-CYC1t; TDH3p-APT1-DIT1t; TEF1p-PRS5-PRM9t; TEF1p-LiMAT-PRM9; TDH3p-TPO5-CYC1t; TEF1p-SPE2-SPE3-PRM9t.

All the native genetic parts, i.e., native promoters, terminators, ORFs and homologous arms, were PCR amplified using CEN.PK113-11C genomic DNA as a template. For optimized heterologous genes, synthetic fragments or plasmids (obtained from GenScript) used for PCR amplification. High-fidelity Phusion DNA polymerase utilized throughout the entire molecular cloning procedure. Cassettes or plasmids introduced into the yeast by the standard LiAc/SS DNA/PEG transformation method. Strains containing URA3-based plasmids or cassettes selected on synthetic complete media without uracil medium (SC-URA), which consisted of 6.7 g/l yeast nitrogen base (YNB) without amino acids, 0.77 g/l complete supplement mixture without uracil (CSM-URA), 20 g/l glucose and 20 g/l agar. The URA3 maker was removed and selected against on 5-fluoroorotic acid (5′-FOA) plates. Moreover, CRISPR/cas9 based system was also used to practice the deletions of AAH1, SPE4 and FMS1. The other gene knockout experiments were conducted by the traditional methods. All the primers used herein are listed in Table 1, all plasmids are listed in Table 2, and all strains are listed in Table 3.

TABLE 1
primers

| | | SEQ ID |
|---|---|---|
| Oligo Name | Sequence 5′ to 3′ | NO: |
| 001_OAZ1- | GTATGAAGTAATACAGAAAAGGAAAAC | 44 |
| L-F1 | | |
| OAZ1-L-R1 | GACCTGCAGCGTACGAAGCTTCAGCAATCTCTGGTGGAACGTCTAG | 45 |
| (Kan) | | |
| KanMX-F1 | CTGAAGCTTCGTACGCTGCAGGTC | 46 |
| KanMX-R1 | GGCCACTAGTGGATCTGATATCAC | 47 |
| OAZ1-R- | GTGATATCAGATCCACTAGTGGCCCCTGAACCAAAACTTCAAATTCGAAT | 48 |
| F1(kan) | AC | |
| OAZ1-R-R1 | GCATTCAAACTCTAAAATAACAAAG | 49 |
| TEF1P- | GTGATATCAGATCCACTAGTGGCCATAGCTTCAAAATGTTTCTACTCC | 50 |
| F1(kan) | | |
| 008_TEF1p- | TTTGTAATTAAAACTTAGATTAGATTGC | 51 |
| R1 | | |
| PR9_SPE1- | GCAATCTAATCTAAGTTTTAATTACAAAATGTCTAGTACTCAAGTAGGAAA | 52 |
| F1(TEF) | TGC | |
| PR10_SPE1- | GTGCTAGTGTCTCCCGTCTTCTGTTCAATCGAGTTCAGAGTCTATGTATA | 53 |
| R1(PRM9) | C | |
| PR_11_ | ACAGAAGACGGGAGACACTAGCAC | 54 |
| PRM9t-F1 | | |
| 12_PRM9t- | ATTTTCAACATCGTATTTTCCGAAGC | 55 |
| R1 | | |
| OAZ1-R- | GCTTCGGAAAATACGATGTTGAAAATCCTGAACCAAAACTTCAAATTCGA | 56 |
| F2(PRM9) | ATAC | |
| pyx212t- | GTGATATCAGATCCACTAGTGGCCTGCCGTAAACCACTAAATCGGAACC | 57 |
| R1(kan) | C | |
| 15_pyx212t- | TAGGGCCCACAAGCTTACGCGTCGAC | 58 |
| F1 | | |
| SPE3- | GTCGACGCGTAAGCTTGTGGGCCCTACTAATTTAATTCCTTGGCTGCCC | 59 |
| R1(pyx212t) | AG | |
| SPE3- | CTACTTTTTACAACAAATATAACAAAATGGCACAAGAAATCACTCACCCA | 60 |
| F1(PGK) | AC | |
| 18_PGK1p- | TTTGTTATATTTGTTGTAAAAAGTAG | 61 |
| R1 | | |
| PGK1p-F1 | ACGCACAGATATTATAACATCTGCAC | 62 |
| TEF1p- | GTGCAGATGTTATAATATCTGTGCGTATAGCTTCAAAATGTTTCTACTCC | 63 |
| F2(PGK1p) | | |
| DIT1t- | CTTCGGAAAATACGATGTTGAAAATGTTACTCCGCAACGCTTTTCTGAAC | 64 |
| R1(PRM9t) | G | |
| PR22_DIT1t- | TAAAGTAAGAGCGCTACATTGGTCTACC | 65 |
| F1 | | |
| SPE2- | GGTAGACCAATGTAGCGCTCTTACTTTATCATATTTTCTTCTGCAATTTCA | 66 |
| R1(DIT1t) | TATAG | |
| SPE2- | GTTTCGAATAAACACACATAAACAAACAAAATGACTGTCACCATAAAAGA | 67 |
| F1(TDH3p) | ATTGAC | |
| PR25_ | TTTGTTTGTTTATGTGTGTTTATTCGAAAC | 68 |
| TDH3p-R1 | | |
| 26_TDH3p- | TCGAGTTTATCATTATCAATACTGCC | 69 |
| F1 | | |
| OAZ1-R- | GGCAGTATTGATAATGATAAACTCGACCTGAACCAAAACTTCAAATTCGA | 70 |
| F3(TDH3p) | ATAC | |
| TPIp- | GGCAGTATTGATAATGATAAACTCGAGTTTAAAGATTACGGATATTTAAC | 71 |
| F1(TDH3p) | TTAC | |
| PR29_TPIp- | TTTTAGTTTATGTATGTGTTTTTTGTAGTTATAG | 72 |
| R1 | | |
| SPE4- | CTATAACTACAAAAAACACATACATAAACTAAAAATGGTTAATAATTCACA | 73 |
| F1(TPIp) | GCATCCTTAC | |
| SPE4- | GACTAATAATTCTTAGTTAAAAGCACTTCATTCATTAATGACCTTGTCTGC | 74 |
| R1(ENO2t) | CC | |
| ENO2t-F1 | AGTGCTTTTAACTAAGAATTATTAGTC | 75 |
| ENO2t-R1 | AGGTATCATCTCCATCTCCCATATGC | 76 |
| OAZ1-R- | GCATATGGGAGATGGAGATGATACCTCCTGAACCAAAACTTCAAATTCG | 77 |
| F4(ENO2t) | AATAC | |
| (ZWM)TPIp- | CGTCCTCTCGAAAGGTGGTTTAAAGATTACGGATATTTAACTTAC | 78 |
| F-156 | | |
| (TPIp)MEU1- | CTACAAAAAACACATACATAAACTAAAAATGAACAGGATTAAGAATACATT | 79 |
| F-157 | TTCTG | |
| MEU1- | GGTCGACGCGTAAGCTTGTGGGCCCTATTACCAATAGTTTGGAAATAAG | 80 |
| R(Pyx212t)- | TATC | |
| 158 | | |
| Pyx212t- | CAGGTGGTCATGGCCCTTTGCCGTAAACCACTAAATCG | 81 |
| R(ZWM)- | | |
| 159 | | |
| (TPIp)BAT2- | CTACAAAAAACACATACATAAACTAAAAATGACCTTGGCACCCCTAGAC | 82 |
| F-160 | | |
| BAT2- | GGTCGACGCGTAAGCTTGTGGGCCCTATCAGTTCAAATCAGTAACAACC | 83 |
| R(Pyx212t)- | | |
| 161 | | |
| MEU1- | CATTAAAAAACTATATCAATTAATTTGAATTAACTTACCAATAGTTTGGAA | 84 |
| R(FBA1t)- | ATAAGTATC | |
| 162 | | |
| BAT2- | CATGACTCGAGGTCGACGGTATCTCAGTTCAAATCAGTAACAACCCTTG | 85 |
| R(CYC1t)- | | |
| 163 | | |
| BAT2- | GTAATTATCTACTTTTTACAACAAATATAACAAAATGACCTTGGCACCCCT | 86 |
| F(PGK1p)- | AGAC | |
| 164 | | |
| TEF1p- | CAGGTGGTCATGGCCCTTTTTGTAATTAAAACTTAGATTAGATTGC | 87 |
| R(ZWM)- | | |
| 165 | | |
| (TEF1p) | GCAATCTAATCTAAGTTTTAATTACAAAATGTCCAAGAGCAAAACTTTCTT | 88 |
| SAM2-F-166 | ATTTACC | |
| SAM2- | CTAAATCATTAAAGTAACTTAAGGAGTTAAATTTAAAATTCCAATTTCTTT | 89 |
| R(TDH2t)- | GG | |
| 167 | | |
| TDH2t-F- | ATTTAACTCCTTAAGTTACTTTAATGATTTAG | 90 |
| 168 | | |
| ADH1t- | CAGGTGGTCATGGCCCTTGCGAATTTCTTATGATTTATG | 91 |
| F(ZWM)-169 | | |
| ADH1t-F- | GCGAATTTCTTATGATTTATG | 92 |
| 170 | | |
| APT1- | CATAAATCATAAGAAATTCGCTCATTTTTTCAACGCTTCCTTTTGAGC | 93 |
| R(ADH1t)- | | |
| 171 | | |
| APT1- | CGAATAAACACACATAAACAAACAAAATGTCTATAGCAAGTTATGCCCAA | 94 |
| F(TDH3p)- | G | |
| 172 | | |
| tHXT7p- | CAGGTGGTCATGGCCCTTTTTTTGATTAAAATTAAAAAAACTTTTTG | 95 |
| R(ZWM)- | | |
| 173 | | |
| tHXT7p-R- | TTTTTGATTAAAATTAAAAAAACTTTTTG | 96 |
| 174 | | |
| PRS5- | CAAAAAGTTTTTTTAATTTTAATCAAAAAATGTCAATGAGTAATATTGTTGT | 97 |
| F(THXT7p)- | TTTTGG | |
| 175 | | |
| (ZWM)FBA1t | CGTCCTCTCGAAAGGTGGTTAATTCAAATTAATTGATATAGTTTTTTAATG | 98 |
| (F)-176 | | |
| p423GPD1- | AAGGGCCATGACCACCTGATGCACCAATTAGGTAGGTCTGGCTATGTCT | 99 |
| F1-177 | ATACCTCTGGCAATTCGCCCTATAGTGAGTCG | |
| p423GPD1- | CACCTTTCGAGAGGACGATGCCCGTGTCTAAATGATTCGACCAGCCTAA | 100 |
| R1-178 | GAATGTTCAACGAGCTCCAGCTTTTGTTCCCTTTAGTG | |
| (ZWM) | GCATCGTCCTCTCGAAAGGTGTCGAGTTTATCATTATCAATACTGCC | 101 |
| TDH3p-179 | | |
| TDH2t-F1- | ATTTAACTCCTTAAGTTACTTTAATGATTTAG | 102 |
| 182 | | |
| ADH1t-F1- | GCGAATTTCTTATGATTTATG | 103 |
| 183 | | |
| TPI1t-F1- | GATTAATATAATTATATAAAAATATTATCTTCTTTTC | 104 |
| 188 | | |
| PAA1-L-F- | CTGGTTTGTTTTACAACCAAAAG | 105 |
| 211 | | |
| PAA1-L- | CAACTCTTTCATAAAATGGTATCTTTAACTTTTTATTTAATCGTAATG | 106 |
| R(LINKER)- | | |
| 212 | | |
| Verify- | CAATGACAAGTTTCATCATC | 107 |
| PAA1-F-216 | | |
| 277- | GCAATCTAATCTAAGTTTTAATTACAAAGTTACTCCGCAACGCTTTTCTGA | 108 |
| (TEF1p) | ACG | |
| DIT1t-R | | |
| 278- | GCAATCTAATCTAAGTTTTAATTACAAAATGAACAGGATTAAGAATACATT | 109 |
| (TEF1p)- | TTCTG | |
| MEU1-F | | |
| 279-MEU1- | CTAGTGTCTCCCGTCTTCTGTTTACCAATAGTTTGGAAATAAGTATC | 110 |
| R(PRM9t) | | |
| 280-(TPIp)- | CTATAACTACAAAAAACACATACATAAACTAAAAATGACCTTGGCACCCC | 111 |
| BAT2-F | TAGAC | |
| 281-BAT2- | GACTAATAATTCTTAGTTAAAAGCACTTCAGTTCAAATCAGTAACAACC | 112 |
| R(ENO2t) | | |
| 282TaSPDS- | GTCGACGCGTAAGCTTGTGGGCCCTATCATGATGCTGTAATAGCAGAAT | 113 |
| R1(pyx212t) | C | |
| 283(PGK) | CTACTTTTTACAACAAATATAACAAAATGGAAGGTGGTGGTGCTAGAAAT | 114 |
| TaSPDS-F1 | G | |
| 290TPO5- | CTATAACTACAAAAAACACATACATAAACTAAAAATGCCAGAGTATACGC | 115 |
| F1(TPIp) | TACTG | |
| 291TPO5- | GACTAATAATTCTTAGTTAAAAGCACTTCATATATCATATCTACGATCATC | 116 |
| R1(ENO2t) | G | |
| 2920AZ1- | GTGAAGGAACAACTCGTGTCTC | 117 |
| F(Veri) | | |
| 2930AZ1- | GAGGATACGTACATATGCAAGC | 118 |
| R(Veri) | | |
| 294_ | TGCCGTAAACCACTAAATCGGAACC | 119 |
| pYX212t-R | | |
| 295-CAN1- | TCAGACTTCTTAACTCCTGTAAAAACAAAAAAAAAAAAAGGCATAGCAAT | 120 |
| FW | AAGCTGGAGCTCATAGCTTC | |
| 296-CYC1t- | GTGCCTATTGATGATCTGGCGGAATGTCTGCCGTGCCATAGCCATGCCT | 121 |
| tagA-RV | TCACATATAGTCCGCAAATTAAAGCCTTCGAG | |
| 297-tagA- | ACTATATGTGAAGGCATGGCTATGGCACGGCAGACATTCCGCCAGATCA | 122 |
| PUG | TCAATAGGCACCTTCGTACGCTGCAGGTCGAC | |
| 315_SPE4_ | TGCGCATGTTTCGGCGTTCGAAACTTCTCCGCAGTGAAAGATAAATGAT | 123 |
| targetRNA | CACTCATCAAATTCCGTACATGTTTTAGAGCTAGAAATAGCAAGTTAAAA | |
| FW | TAAGGCTAGTCCGTTATCAAC | |
| 316_SPE4_ | GTTGATAACGGACTAGCCTTATTTTAACTTGCTATTTCTAGCTCTAAAACA | 124 |
| targetRNA | TGTACGGAATTTGATGAGTGATCATTTATCTTTCACTGCGGAGAAGTTTC | |
| RV | GAACGCCGAAACATGCGCA | |
| 317_SPE4_ | ATAAAATATCAAAACGCCGATGAGACAGGCAGGATAAAGTGACAGATTC | 125 |
| repair oligo | AGTTATACATTTTTATTAGCATTGATATTATTATTTTAAAAAGTCTATTTAC | |
| fw | TTGTATATTTATCCGAATA | |
| 318_SPE4_ | TATTCGGATAAATATACAAGTAAATAGACTTTTTAAAATAATAATATCAAT | 126 |
| repair oligo | GCTAATAAAAATGTATAACTGAATCTGTCACTTTATCCTGCCTGTCTCATC | |
| rv | GGCGTTTTGATATTTTAT | |
| 319_SPE4_ | TTTTGCAACATCCGGGCATG | 127 |
| dg fw | | |
| 320_SPE4_ | CGGCTAGCTGGTATGGATCG | 128 |
| dg rv | | |
| 321_TEF1p_ | ATAGCTTCAAAATGTTTCTACTCC | 129 |
| FW | | |
| 322_X-2- | TGCGACAGAAGAAAGGGAAG | 130 |
| Diag_FW | | |
| 323_X_2_ | CGTCTATGAGGAGACTGTTAG | 131 |
| UP_FW | | |
| 324_X_2_ | GGAGTAGAAACATTTTGAAGCTATACGTGACCACTTCGAGAGC | 132 |
| UP_RV(TEF1p) | | |
| 325_SPE2_ | GCAATCTAATCTAAGTTTTAATTACAAAATGACTGTCACCATAAAAGAATT | 133 |
| FW(TEF1p) | G | |
| 326_SPE2 | GTGCTAGTGTCTCCCGTCTTCTGTTCATATTTTCTTCTGCAATTTCATATA | 134 |
| RV(PRM9t) | G | |
| 327_X_2_ | GCTTCGGAAAATACGATGTTGAAAATCCTGCATAATCGGCCTCAC | 135 |
| DW_FW | | |
| (PRM9t) | | |
| 328_X_2_ | CTCGCCAAGGCATTACCATO | 136 |
| DW_RV | | |
| 329_X-2- | GAGAACGAGAGGACCCAAC | 137 |
| Diag_RV | | |
| 330_ | GGTTCCGATTTAGTGGTTTACGGCAACGTGACCACTTCGAGAGC | 138 |
| X2_UP_RV | | |
| (pYX212t) | | |
| 331_SPE2_ | GTCGACGCGTAAGCTTGTGGGCCCTATCATATTTTCTTCTGCAATTTCAT | 139 |
| RV(pYX212) | ATAG | |
| 332_SPE2_ | CTACTTTTTACAACAAATATAACAAAATGACTGTCACCATAAAAGAATTG | 140 |
| FW(PGK1p) | | |
| 333_APT1_ | GGTAGACCAATGTAGCGCTCTTACTTTATCATTTTTTCAACGCTTCCTTTT | 141 |
| RV(DIT1t) | G | |
| 334_APT1_ | GTTTCGAATAAACACACATAAACAAACAAAATGTCTATAGCAAGTTATGC | 142 |
| FW(TDH3p) | CCAAG | |
| 335_X_2_ | GGCAGTATTGATAATGATAAACTCGACCTGCATAATCGGCCTCAC | 143 |
| DW_FW | | |
| (TDH3p) | | |
| 336_PRS5_ | CTATAACTACAAAAAACACATACATAAACTAAAAATGTCAATGAGTAATAT | 144 |
| FW(TP11p) | TGTTG | |
| 337_PRS5_ | GACTAATAATTCTTAGTTAAAAGCACTCTACCATCCATGCTCGAACAAC | 145 |
| RV(ENO2t) | | |
| 338_X_2_ | GCATATGGGAGATGGAGATGATACCTCCTGCATAATCGGCCTCAC | 146 |
| DW_FW | | |
| (ENO2t) | | |
| 359-CYC1t- | GGGTACCGGCCGCAAATTAAAGCCTTCGAGCGTCCC | 147 |
| RV | | |
| 392-X-4-up- | GTGTTCATTGTACGTC CTAGAC | 148 |
| dg-fw | | |
| 393-X-4-up- | GTGCCCAAAGCTAAGAGTC | 149 |
| fw | | |
| 394-X-4-up- | CTGCTCTTGAATGGCGAC | 150 |
| rv | | |
| 395-(X-4- | GTCGCCATTCAAGAGCAGCATCGTCCTCTCGAAAGGTG | 151 |
| up)YP8-fw | | |
| 396-(X-4- | CGAATCTTCCCATGCCTGCAGGTGGTCATGGCCCTT | 152 |
| down)YP8- | | |
| rv | | |
| 397-X-4- | CAGGCATGGGAAGATTCG | 153 |
| down-fw | | |
| 398-X-4- | CTGGTGAGGATTTACGGTATG | 154 |
| down-rv | | |
| 399-X-4- | GTGCGTTATCGGGTTCTTAC | 155 |
| down-dg-rv | | |
| 400-XI-3-up- | CAGGTTAGTTACT TGCTCTATG | 156 |
| fw | | |
| 401-XI-3-up- | CAGTATTGATAATGATAAACTCGAAATCAGACGCACGCTTG | 157 |
| rv(TDH3p) | | |
| 402- | CTTTAATTTGCGGCCGGTACCCTTACGTGGATTGAGCCAG | 158 |
| (CYC1t)XI- | | |
| 3-dow-fw | | |
| 403-XI-3- | GATTGTCATAATAGGAGCTATTTG | 159 |
| dow-rv | | |
| 404-XI-3-up- | CCATAGTATTACTATTGGTGTTCAT | 160 |
| fw(dg) | | |
| 405-XI-3- | GTTATCGGTTGTGATATTGTTC | 161 |
| down-rv(dg) | | |
| 406- | TTAAGCTATTGTTTCGGCAATT | 162 |
| CgargD-rv | | |
| 425-CAR2p- | CGTGCGAUCTCTATAAAAAATGTGCGAAC | 163 |
| fw | | |
| 426-CAR2p- | ATGACAGAUTGGTGTTGTGGTTCTGTG | 164 |
| rv | | |
| 427-X-3-up- | GAGATCTTTGTGTTCGGTTAC | 165 |
| fw | | |
| 428-X-3-up- | AGTCTCGTATGTCGGCTC | 166 |
| rv | | |
| 431-X-3- | TGTGTCCGCGTTTCTAAG | 167 |
| down-fw | | |
| 432-X-3- | GAGGTGGTTATTGATCACCAG | 168 |
| down-rv | | |
| 433-(dg)X-3- | ACGAATCGTTAGGCACAG | 169 |
| up-fw | | |
| 434-(dg)X-3- | GTGCAATACCAAAATCG | 170 |
| down-rv | | |
| 435-GO4- | GCAGTTGTTTGGATTAAAAAGCTGTACG | 171 |
| dg1-fw | | |
| 436-GO4- | CCTTGTGTCATCATTTACTCCAGGC | 172 |
| dg1-rv | | |
| 437-GO4- | GTAGAGTCTTAGCTGCAGTTGGTATG | 173 |
| dg2-fw | | |
| 438-GO4- | CAGGGCATTATTACTGACGGCATG | 174 |
| dg2-rv | | |
| 439-GO4- | CTACAGCACCTTTGAAAGAAGGTGTC | 175 |
| dg3-fw | | |
| 440-GO4- | GTTGATGGTGTCGTAGACGTCAG | 176 |
| dg3-rv | | |
| 441-GO4- | GGAACGTGGATTTACCCCAG | 177 |
| dg4-fw | | |
| 442-GO4- | GTTATCGGTTGTGATATTGTTCCTGC | 178 |
| dg4-rv | | |
| 443-MEU1- | CAAAGCGATGGGCTCCAGAC | 179 |
| dg-rv | | |
| 444-BAT2- | CATTCCGCAGTTAACATGTGGTC | 180 |
| dg-fw | | |
| 445-X-4-up- | GTGTTCATTGTACGTC CTAGACTCAAAC | 181 |
| dg-fw2 | | |
| 446-X-4- | CGTGCGTTATCGGGTTCTTAC | 182 |
| down-dg-rv2 | | |
| 447-(X-4- | GGAACACTGGGGCAATAGGCTGTCGCCATT | 183 |
| up)YP8-fw2 | CAAGAGCAGCATCGTCCTCTCGAAAGGTG | |
| 448-(X-4- | CTATTGTAATTCAAAAAAAAAAAGCGAATCTTCCCATGCCTGCAGGTGGT | 184 |
| down)YP8- | CATGGCCCTT | |
| rv2 | | |
| 454_PGK1p- | CTTGCATAAATTGGTCAATGCAAG | 185 |
| Fw1(dg) | | |
| 455_TEF1p- | CGATGACCTCCCATTGATATTTAAG | 186 |
| Fw1(dg) | | |
| 456_SPE2_ | CATCGTCAATTTGTGATCGAAGAC | 187 |
| Rv1(dg) | | |
| 457_APT1_ | CATTCGCCAGGTAGCTTAC | 188 |
| Rv1(dg) | | |
| 458_PRS5_ | TGCATTTTGAGCGTTGAACAA | 189 |
| Rv1(dg) | | |
| 459_TDH3p_ | GTGCCCTGTTCTCTGTAGTT | 190 |
| Rv1(dg) | | |
| 460_ | ATCGGGCCCTCCTTACTGCTCTCCTTCCGTGTAACGCGTTTGCCGTAAA | 191 |
| pYX212t_v | CCACTAAATCG | |
| (XII-2_up) | | |
| 465_PRM9t_ | CGTTAAGAAAAATTTCGAGAGAGTCGCCGATAGTAGATTTTCAACATCGT | 192 |
| rv(XII- | ATTTTCC | |
| 2_down) | | |
| 466_TEF1p_ | CCTCCTTACTGCTCTCCTTCCGTGTAACGCGTTATAGCTTCAAAATGTTT | 193 |
| fw(XII- | CTACTCC | |
| 2_up) | | |
| 467_XII- | TTATCGAGCT AACTATTTTCGACACACATG | 194 |
| 2_up_fw | | |
| 468_XII- | CGTCGCCCAGTAAGTGAGACTA | 195 |
| 2_down_rv | | |
| 471_PRS5_ | GAAAGCATAGCAATCTAATCTAAGTTTTAATTACAAAATGTCAATGAGTAA | 196 |
| fw(TEF1p) | TATTGTTG | |
| 472_PRS5_ | AAGTTGTGTGCTAGTGTCTCCCGTCTTCTGTCTACCATCCATGCTCGAAC | 197 |
| rv(PRM9t) | A | |
| 488_KEX2p_ | CTCGCCTAGTAAATAAACGATAAACAAATTTGAAGTA | 198 |
| (PFK1p_up)_ | GTAGATACACGTATCTCGACATG | |
| fw | | |
| 489_KEX2p_ | GAATGCAACACCGTAGCATGAATCTTGAGATTGCATCTGATAATGGGTTA | 199 |
| (PFK1p)_rv | GTAGTTTAT | |
| 496_FMS1_ | TCTCCGCAGTGAAAGATAAATGATCAATTTACGAAAAATAAAGGC | 200 |
| targetRNA | GTTTTAGAGCTAGAAATAGCAAGTT | |
| FW | | |
| 497_FMS1_ | AACTTGCTATTTCTAGCTCTAAAACGCCTTTATTTTTCGTAAATTGATCAT | 201 |
| targetRNA | TTATCTTTCACTGCGGAGA | |
| RV | | |
| 498_FMS1_ | AATAAAGGCAAAAACAGTGGTCGTGTGAGAAATCTATTTTTTCGAAATTA | 202 |
| repair oligo | CTTACACTTT | |
| fw | | |
| 499_FMS1_ | AAAGTGTAAGTAATTTCGAAAAAATAGATTTCTCACACGACCACTGTTTTT | 203 |
| repair oligo | GCCTTTATT | |
| rv | | |
| 500_FMS1_ | GGTCACCCACCCATATACGG | 204 |
| dg fw | | |
| 501_FMS1_ | TGTCCTCCGGATAACTGCAC | 205 |
| dg rv | | |
| FMS1_target | TGCGCATGTTTCGGCGTTCGAAACTTCTCCGCAGTGAAAGATAAATGAT | 206 |
| RNA FW | CAATTTACGAAAAATAAAGGCGTTTTAGAGCTAGAAATAGCAAGTTAAAA | |
| | TAAGGCTAGTCCGTTATCAAC | |
| FMS1_target | GTTGATAACGGACTAGCCTTATTTTAACTTGCTATTTCTAGCTCTAAAAC | 207 |
| RNA RV | GCCTTTATTTTTCGTAAATTGATCATTTATCTTTCACTGCGGAGAAGTTTC | |
| | GAACGCCGAAACATGCGCA | |
| FMS1_repair | TCTTTTTTTGTTCCCAACAAGAAGTGAGTTAATAAAGGCAAAAACAGTGG | 208 |
| oligo fw | TCGTGTGAGAAATCTATTTTTTCGAAATTACTTACACTTTTGACGGCTAGA | |
| | AAAGGATATACATACATAT | |
| FMS1_repair | ATATGTATGTATATCCTTTTCTAGCCGTCAAAAGTGTAAGTAATTTCGAAA | 209 |
| oligo rv | AAATAGATTTCTCACACGACCACTGTTTTTGCCTTTATTAACTCACTTCTT | |
| | GTTGGGAACAAAAAAAGA | |
| SPE3_target | TGCGCATGTTTCGGCGTTCGAAACTTCTCCGCAGTGAAAGATAAATGAT | 210 |
| RNA FW | CATCTTCAAATCCACTACATAGTTTTAGAGCTAGAAATAGCAAGTTAAAAT | |
| | AAGGCTAGTCCGTTATCAAC | |
| SPE3_target | GTTGATAACGGACTAGCCTTATTTTAACTTGCTATTTCTAGCTCTAAAACT | 211 |
| RNA RV | ATGTAGTGGATTTGAAGATGATCATTTATCTTTCACTGCGGAGAAGTTTC | |
| | GAACGCCGAAACATGCGCA | |
| SPE3_repair | TTGTACGCTTCACATAGTAGTTCAGTCAAGAAGAGCAAACACTAATAAGC | 212 |
| oligo fw | AATAAATCTAGGAGAATATACATATATATGCATATGTTTGTTTAGCTAAAT | |
| | AATTTTATTGAGCTTTGCT | |
| SPE3_repair | AGCAAAGCTCAATAAAATTATTTAGCTAAACAAACATATGCATATATATGT | 213 |
| oligo rv | ATATTCTCCTAGATTTATTGCTTATTAGTGTTTGCTCTTCTTGACTGAACT | |
| | ACTATGTGAAGCGTACAA | |
| PR524_SPE3_ | ACACCAATATTCTGCACCTGC | 214 |
| dg fw | | |
| PR525_SPE3_ | TGCTGGAGAAGATCGTACGC | 215 |
| dg rv | | |
| SPE2_target | TGCGCATGTTTCGGCGTTCGAAACTTCTCCGCAGTGAAAGATAAATGAT | 216 |
| RNA FW | CTTAGTAGTTTTTGGAAGGATGTTTTAGAGCTAGAAATAGCAAGTTAAAA | |
| | TAAGGCTAGTCCGTTATCAAC | |
| SPE2_target | GTTGATAACGGACTAGCCTTATTTTAACTTGCTATTTCTAGCTCTAAAACA | 217 |
| RNA RV | TCCTTCCAAAAACTACTAAGATCATTTATCTTTCACTGCGGAGAAGTTTC | |
| | GAACGCCGAAACATGCGCA | |
| SPE2_repair | TTCTTTTTTATATTTTTTAGGTTTTCATATAGTGTCTTACGCAAATAGGCG | 218 |
| oligo fw | GACCATAGAAAAGCCGCCATTTGTGTCTCCTCATACTTACATAGAATAGC | |
| | CCTCTTCTATTATCCTTCG | |
| SPE2_repair | CGAAGGATAATAGAAGAGGGCTATTCTATGTAAGTATGAGGAGACACAA | 219 |
| oligo rv | ATGGCGGCTTTTCTATGGTCCGCCTATTTGCGTAAGACACTATATGAAAA | |
| | CCTAAAAAATATAAAAAAGAA | |
| SPE2_dg fw | TACAGCTCGCTCCTTGCATC | 220 |
| SPE2_dg rv | GCTTGCTTGGAGGGCTTTTC | 221 |
| SPE1_target | TGCGCATGTTTCGGCGTTCGAAACTTCTCCGCAGTGAAAGATAAATGAT | 222 |
| RNA FW | CAAGAACCCTTTATCATAATTGTTTTAGAGCTAGAAATAGCAAGTTAAAAT | |
| | AAGGCTAGTCCGTTATCAAC | |
| SPE1_target | GTTGATAACGGACTAGCCTTATTTTAACTTGCTATTTCTAGCTCTAAAACA | 223 |
| RNA RV | ATTATGATAAAGGGTTCTTGATCATTTATCTTTCACTGCGGAGAAGTTTC | |
| | GAACGCCGAAACATGCGCA | |
| SPE1_repair | TTTTTTGATTGTTCTACAACTTTTTCATAGTAATCAAAACCTTTGAATTTCA | 224 |
| oligo fw | AACTTACTAGGATATATTTAACCACGACTTTCGCAAGAGAGACGGAGGG | |
| | GGTGGGAAAAGGCTGAATG | |
| SPE1_repair | CATTCAGCCTTTTCCCACCCCCTCCGTCTCTCTTGCGAAAGTCGTGGTT | 225 |
| oligo rv | AAATATATCCTAGTAAGTTTGAAATTCAAAGGTTTTGATTACTATGAAAAA | |
| | GTTGTAGAACAATCAAAAAA | |
| SPE1_dg fw | TCATCCAGGTTTCAGCACGG | 226 |
| SPE1_dg rv | AGCTCGAACAAGGTGTCAGG | 227 |
| 550_(XI- | GATTACTTACCAATGTGCCATAAACTCCGTGCAC | 228 |
| 5_up)TEF1p- | CAATAGCTTCAAAATGTTTCTACTCC | |
| fw | | |
| 551_PRM9t | CTTGGGTTGTGGGCAATTGGGTGTACTATGAAGCATTTTCAACATCGTAT | 229 |
| (XI-5_dw) | TTTCCGAAGC | |
| 552_(TEF1p) | GCAATCTAATCTAAGTTTTAATTACAAAATGGCACAAGAAATCACTC | 230 |
| SPE3-fw | | |
| 553_SPE3 | GTGCTAGTGTCTCCCGTCTTCTGTCTAATTTAATTCCTTGGCTGC | 231 |
| (PRM9t)-rv | | |
| 554_XI-5- | GACCATCACTAAAGCTTCTCTCTTA | 232 |
| fw(dg) | | |
| 555_XI-5- | TTGAGCAATTCATCGACAACAAGAG | 233 |
| rv(dg) | | |
| 556_SPE2 | AGAACCAGAACCAGATCCTATTTTCTTCTGCAATTTCATATAG | 234 |
| (SPE3)-rv | | |
| 557_(SPE2) | GGATCTGGTTCTGGTTCTATGGCACAAGAAATCACTC | 235 |
| SPE3-fw | | |
| 558_SPE3 | AGAACCAGAACCAGATCCATTTAATTCCTTGGCTGC | 236 |
| (SPE2)-rv | | |
| 559_(SPE3) | GGATCTGGTTCTGGTTCTATGACTGTCACCATAAAAGAATTG | 237 |
| SPE2-fw | | |
| 560_pYX21 | GATTACTTACCAATGTGCCATAAACTCCGTGCAC | 238 |
| 2t(XI-5)-rv | CATGCCGTAAACCACTAAATCGGAAC | |
| AAH1_target | TGCGCATGTTTCGGCGTTCGAAACTTCTCCGCAGTGAAAGATAAATGAT | 239 |
| RNA FW | CTTCTTAGATTACTATTATATGTTTTAGAGCTAGAAATAGCAAGTTAAAAT | |
| | AAGGCTAGTCCGTTATCAAC | |
| AAH1_target | GTTGATAACGGACTAGCCTTATTTTAACTTGCTATTTCTAGCTCTAAAACA | 240 |
| RNA RV | TATAATAGTAATCTAAGAAGATCATTTATCTTTCACTGCGGAGAAGTTTC | |
| | GAACGCCGAAACATGCGCA | |
| AAH1_repair | ATAGTTATTTTGAAATAATAACTACCATTAGAACTAACAAAAGAAAAGAAA | 241 |
| oligo fw | AAAAAAATATACCATTTGCAAGACATTGTATAATATTTTTGTTGAAAGTCT | |
| | TTTTCGATTCATAAGCGC | |
| AAH1_repair | GCGCTTATGAATCGAAAAAGACTTTCAACAAAAATATTATACAATGTCTT | 242 |
| oligo rv | GCAAATGGTATATTTTTTTTTTCTTTTCTTTTGTTAGTTCTAATGGTAGTTA | |
| | TTATTTCAAAATAACTAT | |
| AAH1_dg fw | CTCATCGCATGCCAACGAAG | 243 |
| AAH1_dg rv | GCAGCAAAGCCAACCCTTAC | 244 |
| 579_(TEF1p) | GCAATCTAATCTAAGTTTTAATTACAAAATGTCTGTTCACTCTATCTTG | 245 |
| LiMAT-fw | | |
| 580_LIMAT | GTGCTAGTGTCTCCCGTCTTCTGT TTATTCAACCATCTTCTTTGG | 246 |
| (PRM9t)-rv | | |
| 595_ARG3 | CAATGTAGCGCTCTTACTTTATTATTTCAAGTCCTTGAAATTACC | 247 |
| rv(DIT1t)2 | | |
| 602_GAPp_ | CTGGAGCTCAGTTTATCATTAT | 248 |
| Fw | | |
| 603_CYC1t_ | ACTATAGGGCGAATTGGGTAC | 249 |
| Rv | | |
| 604-X-3-up- | ATAATGATAAACTGAGCTCCAGAGTCTCGTATGTCGGCTC | 250 |
| Rv | | |
| 605-X-3- | GTACCCAATTCGCCCTATAGTTGTGTCCGCG TTTCTAAG | 251 |
| down-Fw | | |
| 610_LIMAT_ | GGTAGACCAATGTAGCGCTCTTACTTTATTATTCAACCATCTTCTTTGGA | 252 |
| rv(DIT1t) | AC | |
| 611_(TDH3p) | GTTTCGAATAAACACACATAAACAAACAAAATGTCTGTTCACTCTATCTTG | 253 |
| LiMAT_fw | T | |
| 612_(TPI1p) | CTATAACTACAAAAAACACATACATAAACTAAAAATGCCGTTTGGAATAG | 254 |
| NCP1_fw | ACAACAC | |
| 613_NCP1 | GACTAATAATTCTTAGTTAAAAGCACTTTACCAGACATCTTCTTGGTATC | 255 |
| rv(ENO2t) | | |
| 618_(dg) | TGGTCACACAACTTGTCTG | 256 |
| LiMAT_rv | | |
| 619_(dg) | GGTACTGGTGGTTTCACTTG | 257 |
| LiMAT_fw | | |
| 622_XII-5- | GTAGTGATCATTGGCTTAACG | 258 |
| up_fw | | |
| 623_XII-5- | GTTCCGATTTAGTGGTTTACGGCAGTGACAATAAATTCAAACCGGT | 259 |
| up(Pyx212t) | | |
| rv | | |
| 624_(PRM9t) | GCTTCGGAAAATACGATGTTGAAAATCAACTCAGAAGTTTGACAGC | 260 |
| XII-5- | | |
| dw_fw | | |
| 625_XII-5- | TCGTTAGATTCTGTATCCCTA | 261 |
| dw_rv | | |
| 626_XII-5- | GGAGTAGAAACATTTTGAAGCTATGTGACAATAAATTCAAACCGGT | 262 |
| up(TEF1p)_ | | |
| rv | | |
| 635_ | ATTTGTGATCGAAGACGAAGAG | 263 |
| (dg)SPE2-rv | | |
| 636_ | TCAAGAAGCCACTACGTG | 264 |
| (dg)SPE3-fw | | |
| 637_ | ACTAGAACATTACCATATGTAGTG | 265 |
| (dg)SPE3-rv | | |
| 638_ | CAACTTGGACGTTCTTCTAC | 266 |
| (dg)SPE2-fw | | |

TABLE 2
plasmids

| | | | Yeast | |
|---|---|---|---|---|
| | | Overexpressed | codon | Backbone |
| No. | Plasmids | gene/genes | optimized | plasmid |
| 1 | BvHSS_p426GPD | BvHSS | Yes | p426GPD |
| 2 | SvHSS_p426GPD | SvHSS | Yes | p426GPD |
| 3 | ScSPE4_p426GPD | ScSPE4 | Yes | p426GPD |
| 4 | AtACL5_p426GPD | AtACL5 | Yes | p426GPD |
| 5 | AtSPMS_p426GPD | AtSPMS | Yes | p426GPD |
| 6 | pLAt4CL-AtSHT | AtSHT + At4CL1 | Yes | p426GPD |
| 7 | pLAt4CL-NaDH29 | NaDH29 + | Yes | p426GPD |
| | | At4CL1 | | |
| 8 | pLAt4CL-AtSCT | AtSCT + At4CL1 | Yes | p426GPD |
| 9 | pLAt4CL-NaAT1 | NaAT1 + At4CL1 | Yes | p426GPD |

TABLE 3
strains

| | | Backbone | | Genetic modifications in the |
|---|---|---|---|---|
| No. | Strain | strain | Plasmid | chromosome |
| 0 | JQSPD_AA | CEN.PK | | MATa SUC2 MAL2-8c ura3-52 |
| | | 113-11C | | his3-Δ1 ARG3p::KEX2p car2Δ, |
| | | | | AGC1, ORT1, GDH1, oaz1Δ, |
| | | | | SPE3, SPE1, SPE2, spe4Δ, |
| | | | | CgargJ, EcargB, CgargC, |
| | | | | CgargD, EcargA, MEU1p, BAT2, |
| | | | | APT1, PRS5, Δfms1, Δaah1, |
| | | | | LiMAT, TPO5, SPE2-SPE3 |
| 1 | JQSPD_AA | JQSPD_AA | BvHSS_p426GPD | MATa SUC2 MAL2-8c ura3-52 |
| | (BvHSS_p426GPD) | | | his3-Δ1 ARG3p::KEX2p car2Δ, |
| | | | | AGC1, ORT1, GDH1, oaz1Δ, |
| | | | | SPE3, SPE1, SPE2, spe4Δ, |
| | | | | CgargJ, EcargB, CgargC, |
| | | | | CgargD, EcargA, MEU1p, BAT2, |
| | | | | APT1, PRS5, Δfms1, Δaah1, |
| | | | | LiMAT, TPO5, SPE2-SPE3 |
| 2 | JQSPD_AA | JQSPD_AA | SvHSS_p426GPD | MATa SUC2 MAL2-8c ura3-52 |
| | (SvHSS_p426GPD) | | | his3-Δ1 ARG3p::KEX2p car2Δ, |
| | | | | AGC1, ORT1, GDH1, oaz1Δ, |
| | | | | SPE3, SPE1, SPE2, spe4Δ, |
| | | | | CgargJ, EcargB, CgargC, |
| | | | | CgargD, EcargA, MEU1p, BAT2, |
| | | | | APT1, PRS5, Δfms1, Δaah1, |
| | | | | LiMAT, TPO5, SPE2-SPE3 |
| 3 | JQSPD_AA | JQSPD_AA | ScSPE4_p426GPD | MATa SUC2 MAL2-8c ura3-52 |
| | (ScSPE4_p426GPD) | | | his3-Δ1 ARG3p::KEX2p car2Δ, |
| | | | | AGC1, ORT1, GDH1, oaz1Δ, |
| | | | | SPE3, SPE1, SPE2, spe4Δ, |
| | | | | CgargJ, EcargB, CgargC, |
| | | | | CgargD, EcargA, MEU1p, BAT2, |
| | | | | APT1, PRS5, Δfms1, Δaah1, |
| | | | | LiMAT, TPO5, SPE2-SPE3 |
| 4 | JQSPD_AA | JQSPD_AA | AtSPMS_p426GPD | MATa SUC2 MAL2-8c ura3-52 |
| | (AtSPMS_p426GPD) | | | his3-Δ1 ARG3p::KEX2p car2Δ, |
| | | | | AGC1, ORT1, GDH1, oaz1Δ, |
| | | | | SPE3, SPE1, SPE2, spe4Δ |
| | | | | CgargJ, EcargB, CgargC, |
| | | | | CgargD, EcargA, MEU1p, BAT2, |
| | | | | APT1, PRS5, Δfms1, Δaah1, |
| | | | | LiMAT, TPO5, SPE2-SPE3 |
| 5 | JQSPD_AA | JQSPD_AA | AtACL5_p426GPD | MATa SUC2 MAL2-8c ura3-52 |
| | (AtACL5_p426GPD) | | | his3-Δ1 ARG3p::KEX2p car2Δ, |
| | | | | AGC1, ORT1, GDH1, oaz1Δ, |
| | | | | SPE3, SPE1, SPE2, spe4Δ, |
| | | | | CgargJ, EcargB, CgargC, |
| | | | | CgargD, EcargA, MEU1p, BAT2, |
| | | | | APT1, PRS5, Δfms1, Δaah1, |
| | | | | LiMAT, TPO5, SPE2-SPE3 |
| 6 | JQSPD_AA | JQSPD_AA | pLAt4CL-AtSHT | MATa SUC2 MAL2-8c ura3-52 |
| | (pLAt4CL-AtSHT) | | | his3-Δ1 ARG3p::KEX2p car2Δ, |
| | | | | AGC1, ORT1, GDH1, oaz1Δ, |
| | | | | SPE3, SPE1, SPE2, spe4Δ, |
| | | | | CgargJ, EcargB, CgargC, |
| | | | | CgargD, EcargA, MEU1p, BAT2, |
| | | | | APT1, PRS5, Δfms1, Δaah1, |
| | | | | LiMAT, TPO5, SPE2-SPE3 |
| 7 | JQSPD_AA | JQSPD_AA | pLAt4CL-NaDH29 | MATa SUC2 MAL2-8c ura3-52 |
| | (pLAt4CL-NaDH29) | | | his3-Δ1 ARG3p::KEX2p car2Δ, |
| | | | | AGC1, ORT1, GDH1, oaz1Δ, |
| | | | | SPE3, SPE1, SPE2, spe4Δ, |
| | | | | CgargJ, EcargB, CgargC, |
| | | | | CgargD, EcargA, MEU1p, BAT2, |
| | | | | APT1, PRS5, Δfms1, Δaah1, |
| | | | | LiMAT, TPO5, SPE2-SPE3 |
| 8 | JQSPD_AA | JQSPD_AA | pLAt4CL-AtSCT | MATa SUC2 MAL2-8c ura3-52 |
| | (pLAt4CL-AtSCT) | | | his3-Δ1 ARG3p::KEX2p car2Δ, |
| | | | | AGC1, ORT1, GDH1, oaz1Δ, |
| | | | | SPE3, SPE1, SPE2, spe4Δ, |
| | | | | CgargJ, EcargB, CgargC, |
| | | | | CgargD, EcargA, MEU1p, BAT2, |
| | | | | APT1, PRS5, Δfms1, Δaah1, |
| | | | | LiMAT, TPO5, SPE2-SPE3 |
| 9 | JQSPD_AA | JQSPD_AA | pLAt4CL-NaAT1 | MATa SUC2 MAL2-8c ura3-52 |
| | (pLAt4CL-NaAT1) | | | his3-Δ1 ARG3p::KEX2p car2Δ, |
| | | | | AGC1, ORT1, GDH1, oaz1Δ, |
| | | | | SPE3, SPE1, SPE2, spe4Δ, |
| | | | | CgargJ, EcargB, CgargC, |
| | | | | CgargD, EcargA, MEU1p, BAT2, |
| | | | | APT1, PRS5, Δfms1, Δaah1, |
| | | | | LiMAT, TPO5, SPE2-SPE3 |

An assay combining deep-well scale fermentation with high-performance liquid chromatography (HPLC) used to assess the resulting strain JQSPD_AA. In particular, 24 deep-well batch fermentations of the resulting JQSPD_AA strain for production of polyamines carried out in minimal medium developed by Verduyn et al 1992. Cultures were inoculated from 24 h precultures at an initial OD₆₀₀ of 0.2 with 2 ml minimal medium in 24 deep-well plates and cultivated at 300 rpm, 30° C. for 120 hours. The minimal medium containing 7.5 g/l (NH₄)₂SO₄, 14.4 g/l KH₂PO₄, 0.5 g/l MgSO₄·7H₂O, 20 g/l glucose, 2 ml/l trace metal and 1 ml/l vitamin solutions supplemented with 40 mg/I uracil, 40 mg/I histidine if needed, pH was adjust to 4.5. Sample was prepared by taking 0.1 ml of liquid culture and was subject to hot water (HW) extraction. In the method, we used minimal medium in the deep-well plate's fermentation as the extract context. Tubes containing 0.9 ml of fermentation medium were preheated in a water bath at 100° C. for 10 min. Then, the hot fermentation medium was quickly poured over the 0.1 ml of liquid culture; the mixture was immediately vortexed, and the sample was placed in the water bath. After 30 min, each tube was placed on ice for 5 min. After centrifugation, the supernatant was directly used for derivatization. For derivatization, 0.125 ml of saturated NaHCO₃ solution and 0.25 ml of dansyl chloride solution (5 mg/ml in acetone) were added to 0.25 ml of sample. Then the reaction mixture was incubated at 40° C. for 1 h in the dark with occasional shaking. The reaction was stopped by adding 0.275 ml of methanol. Samples were filtered through a 25 mm syringe filter (0.45 μm Nylon) used for HPLC detection. The following chromatographic condition were used: C18 (100 mm×4.6 mm i.d., 2.6 μm, Phenomenex Kinetex), excitation wavelength 340 nm, emission wavelength 515 nm, sample injecting 1.5 μl, column temperature 40° C., Detector sensitivity 7, acquisition starts at 4.0 min. The mobile phase was water and methanol with the speed of 1 ml/min. The elution program was as follows: 0-5 min 50% to 65% methanol, 5-7.5 min 65% to 75% methanol, 7.5-9.5 min 75% to 87.5% methanol, 9.5-10.5 min 87.5% to 100% methanol, 10.5-11.5 min 100% methanol, 11.5-13.5 min 100% to 50% methanol, 13.5-16 50% methanol.

Strain JQSPD_AA produced Spd titer at the concentration >400 mg/I, significantly increased Spd titer compared to the strains with only partial of the modifications as used herein (see the examples in WO 2016/144247 and WO 2019/013696).

### Example 2: Production of Higher Polyamines in Yeast

Life has evolved diversiform pathways to synthesize structural variants of polyamines. Indeed, while Put and Spd are typically found in most cells as common polyamines, uncommon polyamines, such as sym-homospermidine (Hspd), thermospermine (Tspm), spermine (Spm), branched chain polyamines, and long-chain polyamines (LCPAs) have also been identified in nature. This Example 2 investigated biosynthesis of sym-homospermidine (Hspd), thermospermine (Tspm) and spermine (Spm) by designing and introducing a genetic module (VII) into the Spd platform strain JQSPD_AA from Example 1.

We first set out to heterologously synthesize triamine Hspd, which exist in both plants and bacteria. In plants, Hspd is the first pathway specific intermediate in pyrrolizidine alkaloids biosynthesis, which is formed by homospermidine synthase (plant HSS; EC 2.5.1.45). This enzyme is more specific than bacteria homospermidine synthase (bacteria HSS; EC 2.5.1.44), as the latter cannot use Put as donor of the aminobutyl group. To explore the possibility of both plant and bacteria HSS for Hspd microbial production, genetic sub-modules (VII-a) and (VII-b) designed to biosynthesis of Hspd in yeast encoded the expression of Senecio vernalis SvHSS and Blastochloris viridis BvHSS13 respectively. The sub-modules were introduced as high-copy plasmid SvHSS_p426GPD and BvHSS_p426GPD ordered from GenScipt and harbored the yeast codon-optimized SvHSS gene [SEQ ID NO: 18] and BvHSS gene [SEQ ID NO: 20] respectively into the Spd platform strain JQSPD_AA. The transformation experiment followed the same procedure as in Example 1. The resulting strain JQSPD_AA (SvHSS_p426GPD) and JQSPD_AA (BvHSS_p426GPD) were assayed for Hspd production with the same procedure as descripted in Example 1.

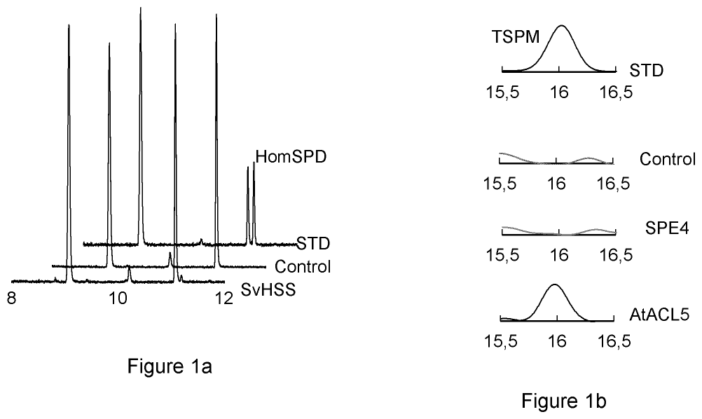

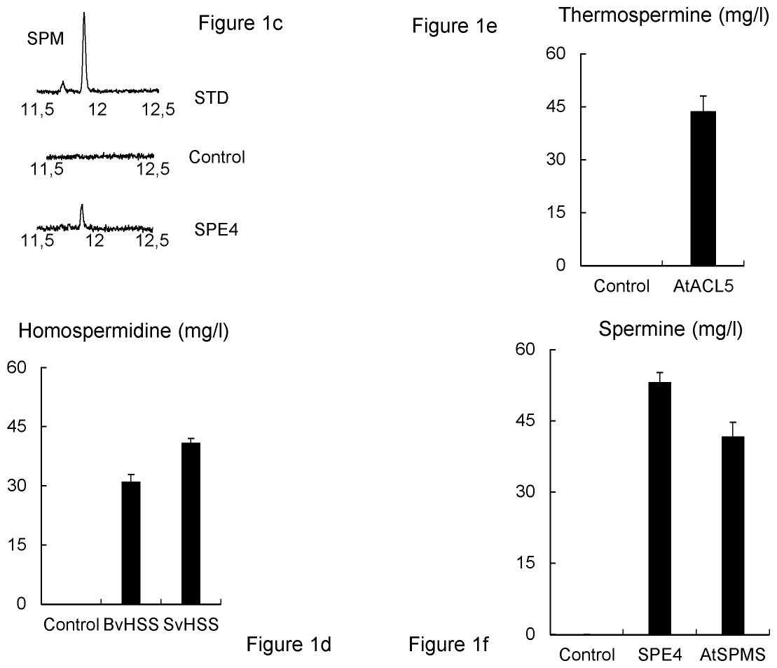

We found that the overexpression of both HSS enabled the biosynthesis of Hspd. In particular, SvHSS enabled Hspd titer at 40.9 mg/, while BvHSS enabled Hspd titer at 31.1 mg/(see FIGS. 1a and 1d).

Subsequently, we also exploited the Spd platform (Example 1) for the production of tetra-amines Spm and Tspm by introducing sub-modules (VII-c), sub-modules (VII-d) and sub-modules (VII-e). Spm is the most common tetra-amine that is found throughout the metazoa, in flowering plants and in yeast. A specific aminopropyltransferase, i.e., spermine synthase (SpmSyn; EC 2.5.1.22) is responsible for Spm biosynthesis. We first explored the yeast native SpmSyn Spe4p for Spm over-production.

When over-expressed codon-optimized yeast SPE4 [SEQ ID NO: 12] as a high-copy plasmid SPE4_p426GPD (sub-module (VII-c)) in JQSPD_AA, 53.1 mg/I of Spm was obtained (see FIGS. 1c and 1f). We also tested the SpmSyn from Arabidopsis thaliana by over-expressed AtSPMS [SEQ ID NO: 14] as a high-copy plasmid AtSPMS_p426GPD (sub-module (VII-d)) in the JQSPD_AA strain. This resulted in production of Spm (41.8 mg/I; see FIGS. 1c and 1f). The plant ACL5 aminopropyltransferase (TspmSyn; EC 2.5.1.79) from A. thaliana was shown to synthesize the Spm isomer Tspm. We thereby also over-expressed AtACL5 [SEQ ID NO: 16] as a high-copy plasmid AtACL5_p426GPD (sub-module (VII-e)) in the JQSPD_AA strain. This strategy enabled 43.8 mg/I of Tspm production (see FIGS. 1b and 1e). All the plasmids harboring the yeast codon-optimized genes were purchased from GenScript. The same transformation and product assay as used in the Example 1 was used in this Example 2. All plasmids are listed in Table 2 and all strains are listed in Table 3.

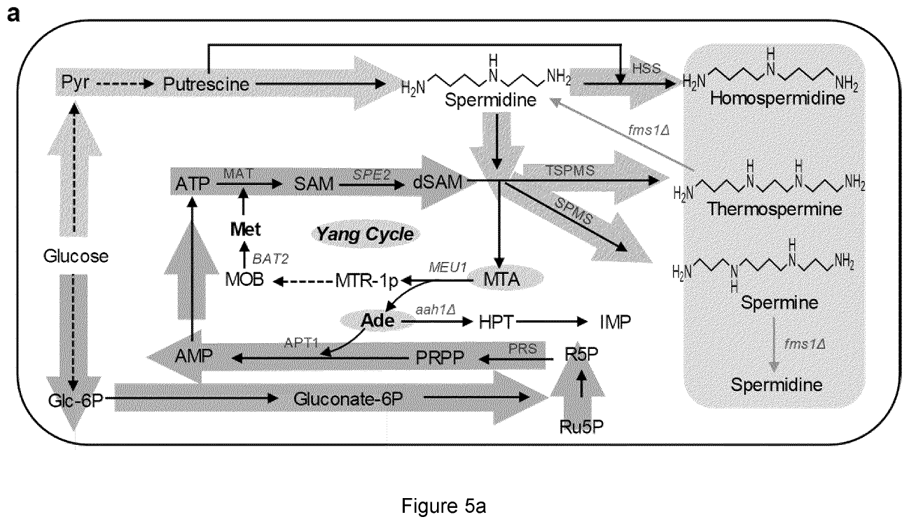

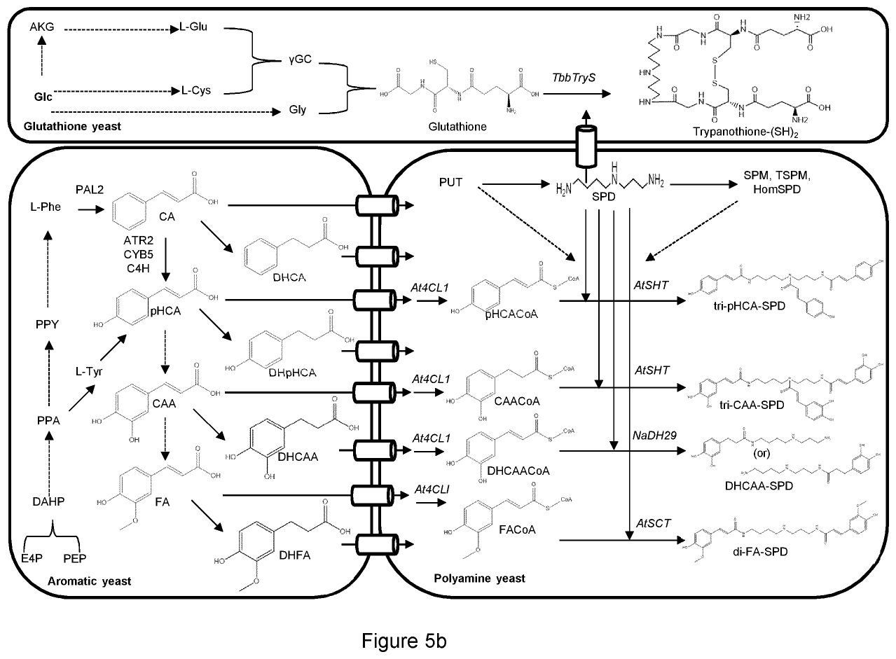

FIG. 5a illustrates engineered pathways for the biosynthesis of spermidine and higher polyamines in yeast.

### Example 3: Biosynthesis of Kukoamines in Yeast

We next set out to synthesize kukoamines, a series of plant polyamine analogs composed of a polymethylenepolyamine backbone, e.g., Put, Spd, and Spm, and at least one dihydrocaffeic acid fragment. Due to their versatile bioactivities, such as antihypertension, antitrypanosome, antilipid peroxidation and lipoxygenase, antisepsis, and neuroprotection, kukoamines have received attention in recent years as functional foods and drug candidates. Kukoamines were first discovered in Cortex Lycii and were then subsequently found in other plants of the Solanaceae family, such as tomato, potato, and tobacco. Coupling of the dihydrocaffeoyl and amine moieties is a committed step and can be considered as the real entry point to the kukoamines biosynthesis. However, the enzymes mediating this reaction in these plants are poorly described so far. Even so, a panel of N-hydrocinnamoyl transferases, which belong to BAHD acyltransferases superfamily, was demonstrated to catalyze N-acylation of polyamines by acylating amine (—NH₂) groups with coenzyme A-activated hydroxycinnamic acid, and their specificity/promiscuity for the acyl acceptor and acyl donor varies, depending on the plant source.

This Example 3 investigated biosynthesis of kukoamines by designing and introducing three genetic sub-modules, expressing multiple N-hydrocinnamoyl transferases into Spd platform strain JQSPD_AA from Example 1. As N-hydrocinnamoyl transferases only accept coenzyme A-activated hydroxycinnamic acids, we also co-expressed an promiscuous 4-coumarate:CoA ligase (EC 6.2.1.12) in these modules. Sub-module (VIII-b) encoded the co-expression of two proteins; an Arabidopsis thaliana promiscuous 4-coumarate:CoA ligase 1 (At4CL1) [SEQ ID NO: 2], which was shown to most efficiently converting caffeic acid into its CoA ester compared to other members of the Arabidopsis thaliana 4CL family; and a spermidine dicoumaroyl transferase (AtSCT; EC 2.3.1.249) [SEQ ID NO: 8]from A. thaliana. This module built as a high-copy plasmid pLAt4CL-AtACT ordered from GenScript, harboring expression cassettes for yeast codon-optimized At4CL1 and AtSCT. All plasmids are listed in Table 2 and all strains are listed in Table 3.

Sub-module (VIII-c) encoded the co-expression of two proteins; the Arabidopsis thaliana promiscuous 4-coumarate:CoA ligase 1 (At4CL1) and a spermidine hydroxycinnamoyl transferase (AtSHT; EC 2.3.1.M34) [SEQ ID NO: 4] from A. thaliana. This module was built as a high-copy plasmid pLAt4CL1-AtSHT ordered from GenScript, harboring expression cassettes for yeast codon-optimized At4CL1 and AtSHT.

Sub-module (VIII-d) encoded the co-expression of two proteins; the Arabidopsis thaliana promiscuous 4-coumarate:CoA ligase 1 (At4CL1) and a spermidine hydroxycinnamoyl transferase (NaDH29; EC 2.3.1.M34) [SEQ ID NO: 6] from tobacco Nicotiana attenuata. This module was built as a high-copy plasmid pLAt4CL1-NaDH29 ordered from GenScript, harboring expression cassettes for yeast codon-optimized At4CL1 and NaDH29.

Last but not least, sub-module (VIII-e) encoded the co-expression of two proteins—the Arabidopsis promiscuous 4-coumarate:CoA ligase 1 (At4CL1) and a N. attenuata putrescine hydroxycinnamoyl transferase (NaAT1; EC 2.3.1.138) [SEQ ID NO: 10]. This module was built as a high-copy plasmid pLAt4CL1-NaAT1 ordered from GenScript, harboring expression cassettes for yeast codon-optimized At4CL1 and NaAT1.

These plasmids were transformed into the Spd platform strain JQSPD_AA with the same procedure as described in Example 2, resulting in strain JQSPD_AA (pLAt4CL-AtSCT), JQSPD_AA (pLAt4CL1-AtAHT), JQSPD_AA (pLAt4CL1-NaDH29) and JQSPD_AA (pLAt4CL1-NaAT1), respectively. These strains were assayed by feeding 2 mM dihydrocaffeic acid (3,4-dihydroxyhydrocinnamic acid) for 120 hours, and the growth medium was analyzed for polyamine analog production according to the following procedures. Detection of polyamine analogs was conducted by liquid chromatography-mass spectrometry (LC-MS) measurements on a Dionex UltiMate 3000 UHPLC (Fisher Scientific, San Jose, CA) connected to an Orbitrap Fusion Mass Spectrometer (Thermo Fisher Scientific, San Jose, CA). The system used an Agilent Zorbax Eclipse Plus C18 2.1×100 mm, 1.8 μm column kept at 35° C. The flow rate was 0.350 mL/min with 0.1% formic acid (A) and 0.1% formic acid in acetonitrile (B) as mobile phase. The gradient started as 5% B for 1 min and then followed a linear gradient to 95% B until 5 min. This solvent composition was held for 1.5 min after which it was changed to 5% B and held until 8 min. The sample (5 μl) was passed on to the MS equipped with a heated electrospray ionization source (HESI) in positive-ion or positive-ion mode with sheath gas set to 50 (a.u.), aux gas to 10 (a.u.) and sweep gas to 1 (a.u.). The cone and probe temperature were 325° C. and 380° C., respectively, and spray voltage was 3500 V. Scan range was 80 to 500 Da and time between scans was 50 ms.

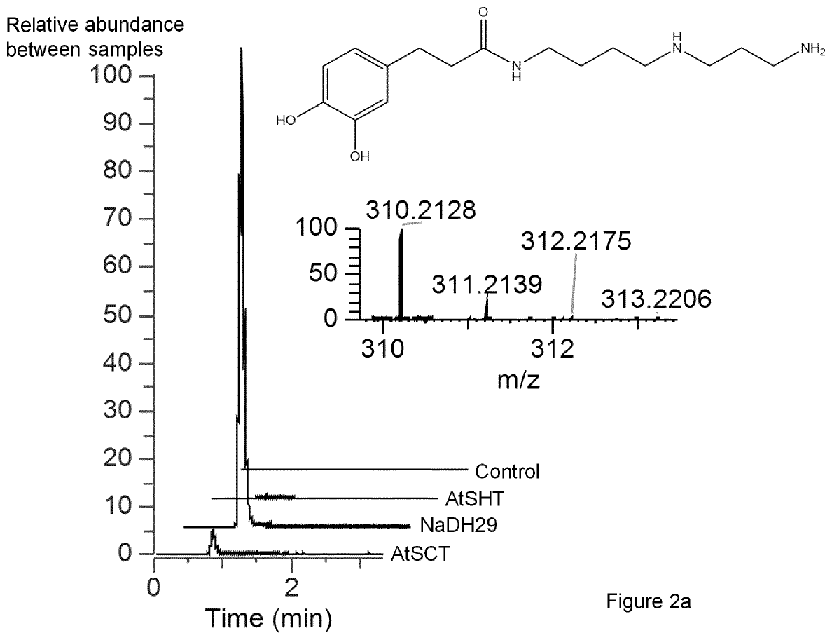

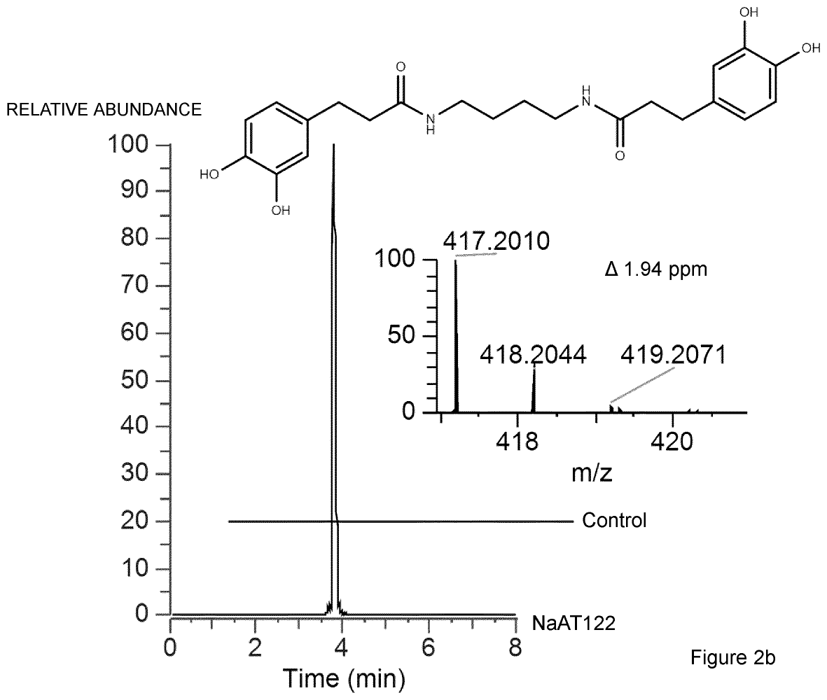

We were happy to see that these efforts led to kukoamines biosynthesis. In particular, a significant single LC-MS peak with a m/z value corresponding to 310.2128 [M+H]⁺ was detected (see FIG. 2a) in the NaDH29 strain, indicating that NaDH29 enabled the biosynthesis of N¹- or N¹⁰-dihydrocaffeoyl spermidine and At4CL1 could also accept dihydrocaffeic acid as a substrate. We could also see a single LC-MS peak with a m/z value corresponding to 310.2128 [M+H]⁺ in the AtSCT overexpressing strain. Moreover, when feeding strain JQSPD_AA (pLAt4CL1-NaAT1) with dihydrocaffeic acid, a significant single LC-MS peak with a m/z value corresponding to 417.2010 [M+H]⁺ was detected (see FIG. 2b), indicating that NaAT1 enabled the biosynthesis of N¹,N⁶-bis(dihydrocaffeoyl)putrescine.

### Example 4: Biosynthesis of Complex Phenolamides in Yeast

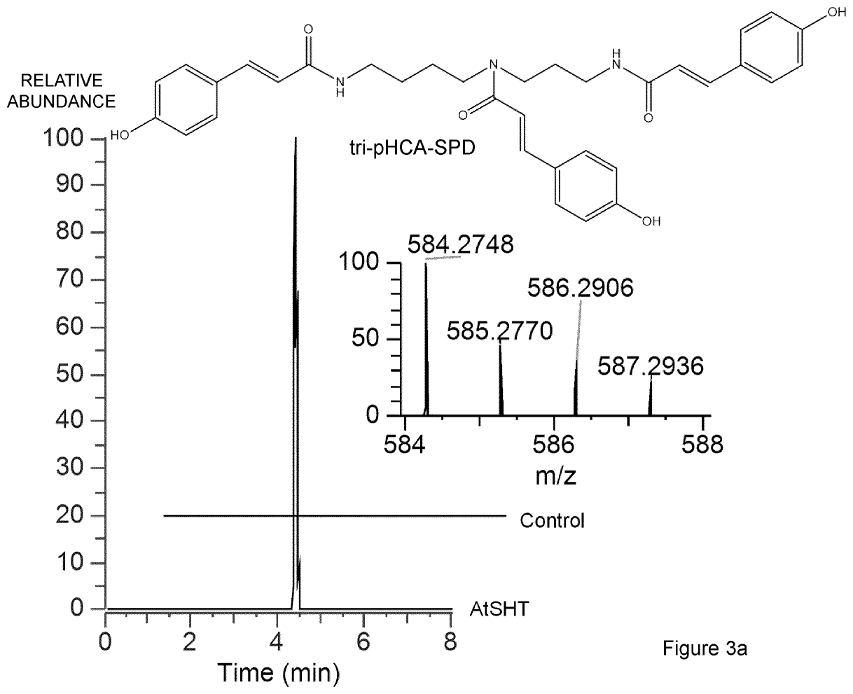

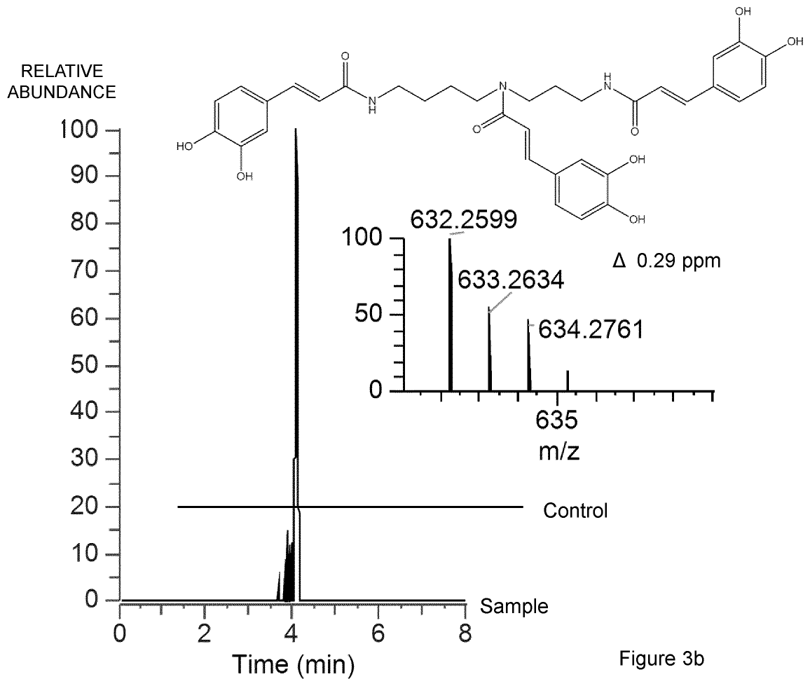

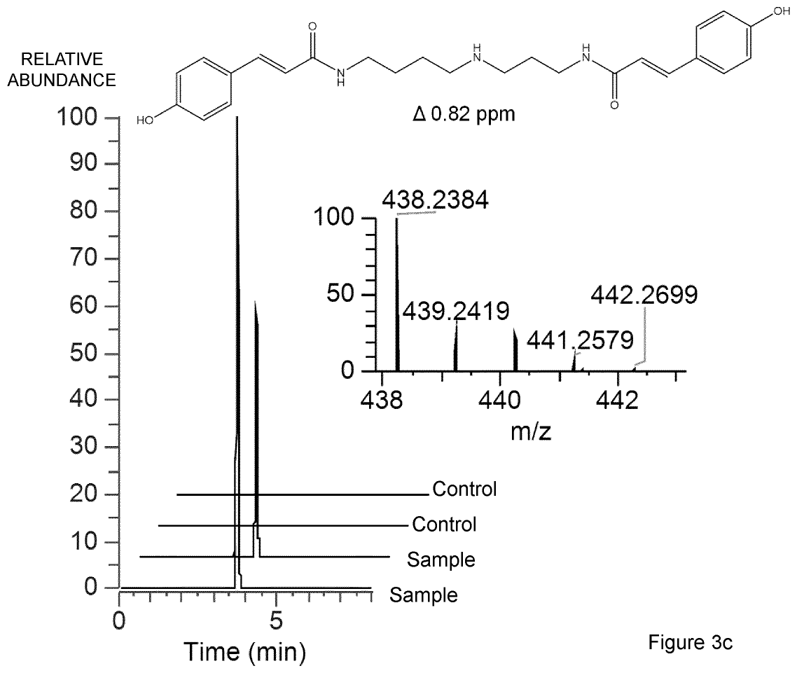

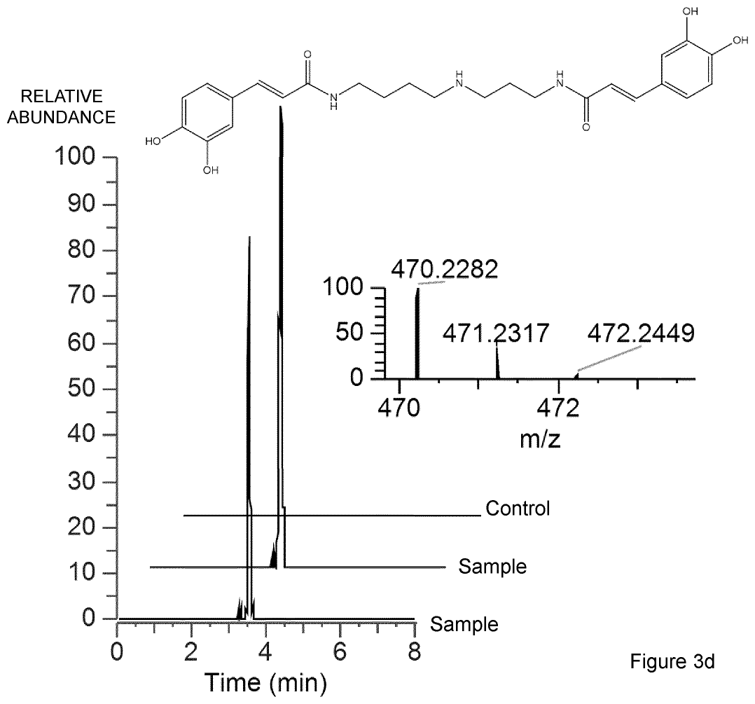

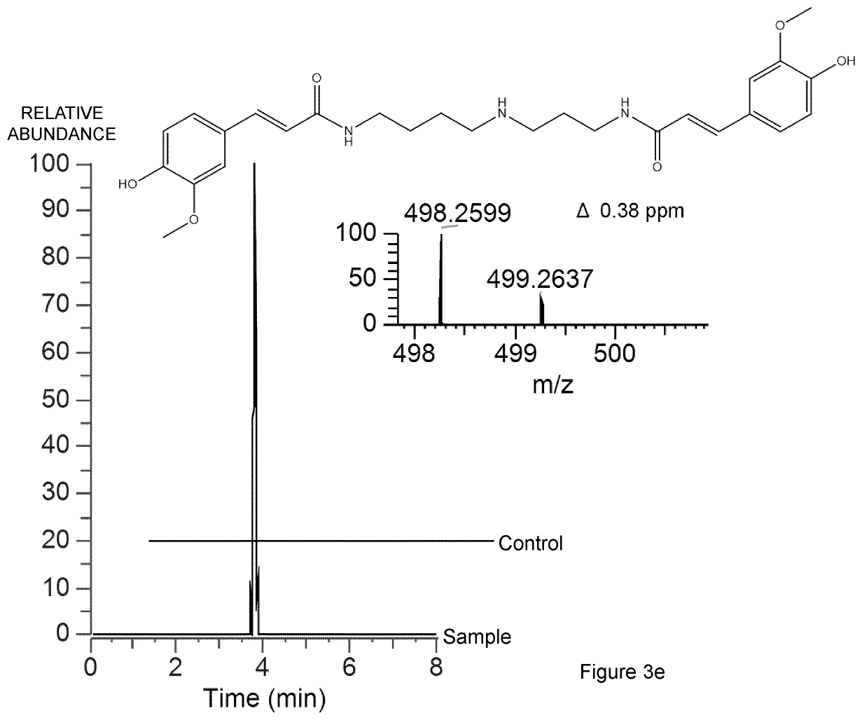

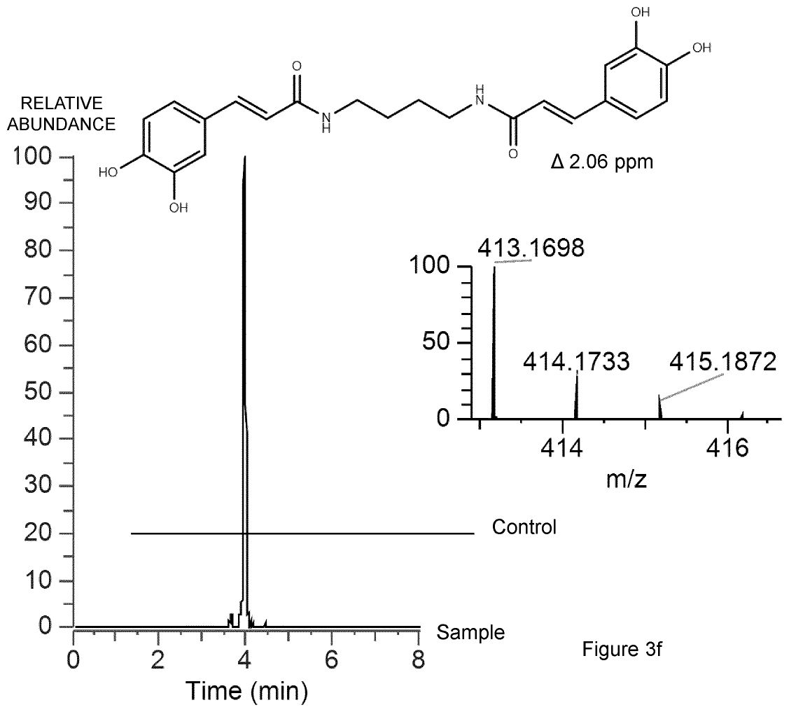

The successful demonstration of biosynthesis of kukoamines in the polyamine platform gave us confidence to further exploit this platform for the biosynthesis of more diverse and complex phenolamides, constituting a quantitatively major group of nitrogen-containing secondary metabolites resulting from the conjugation of a phenolic moiety with polyamines. Thereby, in this Example 4, production of complex phenolamides enabled by over-expressing specific polyamine N-hydrocinnamoyl transferases. Following the same strategy demonstrated in Example 3, we also assayed these strains, i.e., JQSPD_AA (pLAt4CL-AtACT), JQSPD_AA (pLAt4CL1-AtAHT), JQSPD_AA (pLAt4CL1-NaDH29) and JQSPD_AA (pLAt4CL1-NaAT1), by feeding 2 mM p-coumaric acid, 2 mM caffeic acid or 2 mM ferrulic acid for 120 hours and analyzed the medium for the production of polyamine analogs. Fermentation, sample preparation or LC-MS verification procedures were the same as that of Example 3. Indeed, feeding the JQSPD_AA strain co-expressing At4CL1 with AtSCT, AtSHT or NaDH29 with hydroxycinnamic acids, i.e., p-coumaric acid, caffeic acid or ferulic acid, led to phenolamides biosynthesis. In particular, when feeding with p-coumaric acid, a significant single LC-MS peak with a m/z value corresponding to 584.2748 [M+H]⁺ was detected (see FIG. 3a) in the AtSHT strain, indicating that AtSHT enabled the biosynthesis of N¹,N⁵,N¹⁰-tri(coumaroyl) spermidine. Similarly, when feeding with caffeic acid, a significant single LC-MS peak with a m/z value corresponding to 632.2599 [M+H]⁺ was detected (see FIG. 3b) in the AtSHT strain, indicating that AtSHT enabled the biosynthesis of N¹,N⁵,N¹⁰-tri(caffeoyl) spermidine. Furthermore, when feeding with p-coumaric acid, a significant single LC-MS peak with a m/z value corresponding to 438.2383 [M+H]⁺ was detected (see FIG. 3c) in the AtSCT strain, indicating that AtSCT enabled the biosynthesis of N¹,N¹⁰-bis(coumaroyl) spermidine. Similarly, when feeding with caffeic acid, a significant single LC-MS peak with a m/z value corresponding to 470.2282 [M+H]⁺ was detected (see FIG. 3d) in the AtSCT strain, indicating that AtSCT enabled the biosynthesis of N¹,N¹⁰-bis(caffeoyl) spermidine. In addition, when feeding with ferrulic acid, a significant single LC-MS peak with a m/z value corresponding to 498.2599 [M+H]⁺ was detected (see FIG. 3e) in the AtSCT strain, indicating that AtSCT enabled the biosynthesis of N¹,N¹⁰-bis(feruloyl) spermidine. Correspondingly, feeding the NaDH29 strain with p-coumaric acid, caffeic acid or ferulic acid, successfully enabled the biosynthesis of N¹- or N¹⁰-coumaroyl spermidine, N¹- or N¹⁰-caffeoyl spermidine, N¹- or N¹⁰-feruloyl spermidine, respectively. Hence, by choosing different N-hydrocinnamoyl transferases with various regioselectivities, we achieved regioselective biosynthesis of mono-, di-, and tri-substituted spermidine phenolamides. Similarly, feeding the NaAT1 strain with p-coumaric acid, caffeic acid or ferulic acid, successfully enabled the biosynthesis of N¹-coumaroyl putrescine, N¹,N⁶-bis(caffeoyl) putrescine, N¹-caffeoyl putrescine, and N¹-feruloyl putrescine, respectively (see FIG. 3f). All plasmids are listed in Table 2 and all strains are listed in Table 3.

### Example 5: Biosynthesis of Complex Phenolamides in Yeast Co-Cultures

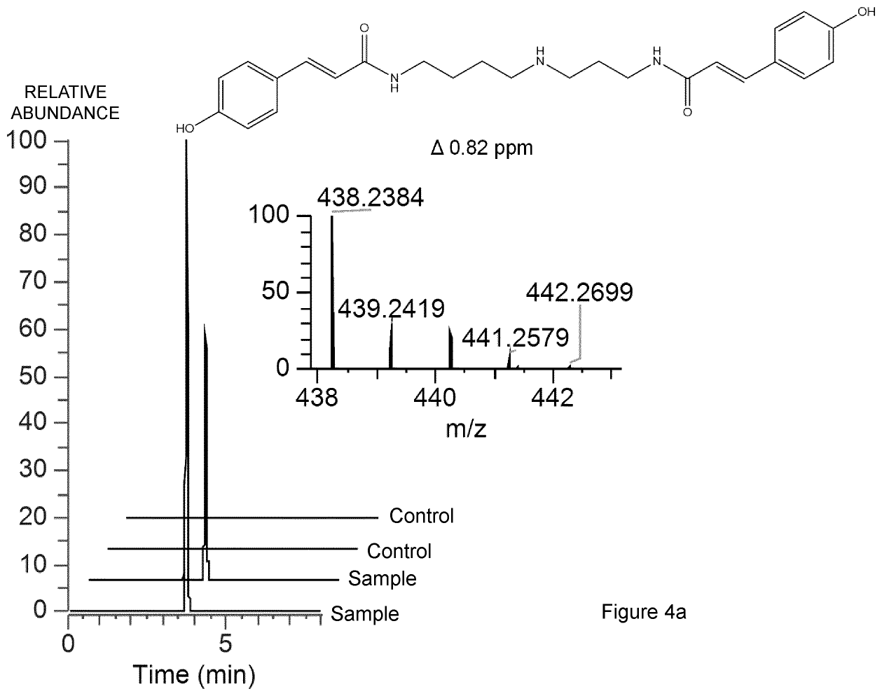

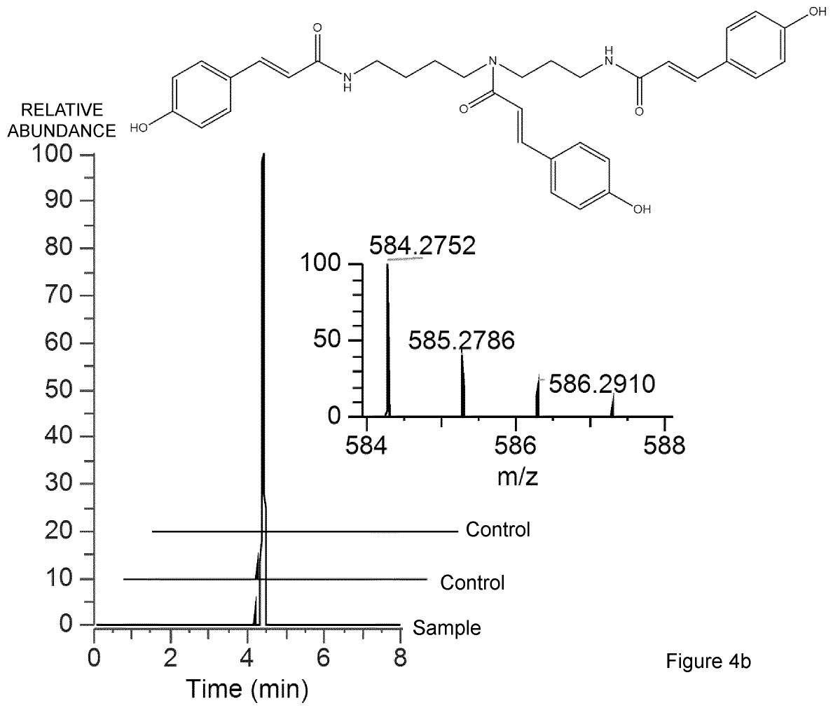

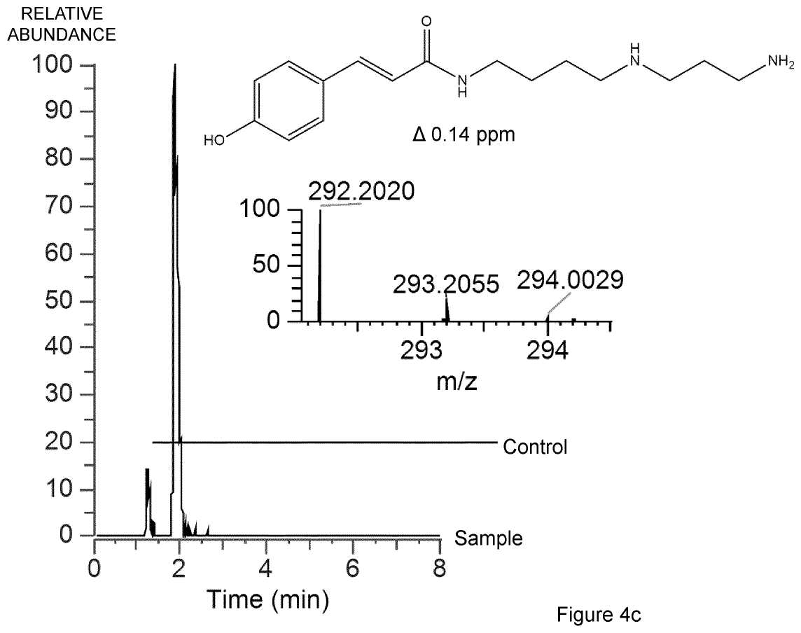

In Example 4, we demonstrated that feeding our polyamine platform strains with multiple aromatic organic acids, for instance, p-coumaric acid, caffeic acid or ferrulic acid enabled the biosynthesis of various polyamine-derived phenolamides. However, these aromatic organic acids used in the titration experiments are generally expensive to obtain, which, to some extent, sacrifice the economic feasibility of this titration-based process for the production of phenolamides. On the contrast, we believe that de novo production of these phenolamides without feeding any aromatic organic acids could be an economically feasible bioprocess. Indeed, recent advances in metabolic engineering and synthetic biology of microbes, including yeast, has already results in many platform strains for the production of theses aromatic organic acids, for instance, p-coumaric acid. To demonstrate the concept of de novo production of polyamine-derived phenolamides with our polyamine platforms, we introduced an extra genetic module sub-module (VIII-f), a p-coumaric acid over-producing yeast strain to our system. We demonstrated this by designing a synthetic consortium comprising a polyamine producing strain and a p-coumaric acid over-producing strain. In particular, we co-cultivated the JQSPD_AA strain co-overexpressing At4CL1 and one of AtSHT, AtSCT, NaDH29 and NaAT1 with p-coumaric acid over-producing stain QL58 (Liu et al., 2019), which resulted in de novo biosynthesis of a series of polyamine-p-coumaric acid conjugates, i.e., N¹,N⁵,N¹⁰-tri(coumaroyl) spermidine, N¹,N¹⁰-bis(coumaroyl) spermidine, N¹- or N¹⁰-coumaroyl spermidine and N¹-coumaroyl putrescine (see FIGS. 4a to 4c). It should be emphasized that, the sub-module (VIII-f) used herein could also, or alternatively, be introduced by means of introducing all the positive genetic targets in the p-coumaric acid over-producing strains into our polyamine platform strains, for instance JQSPD_AA and its derivatives.

FIG. 5b illustrates engineered pathways for the biosynthesis of complex phenolamides in yeast.

### Example 6: Biosynthesis of Halogenated Phenolamides in Yeast Co-Cultures

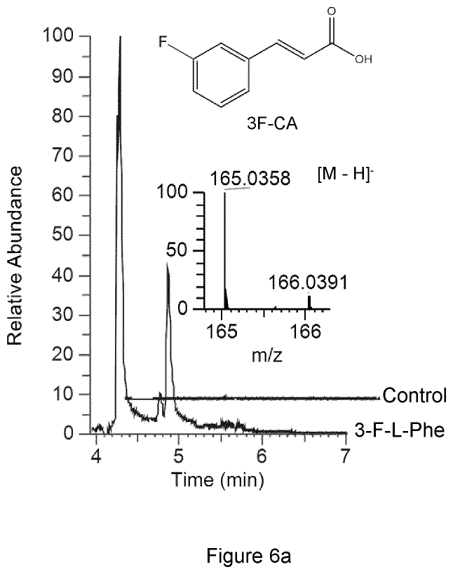

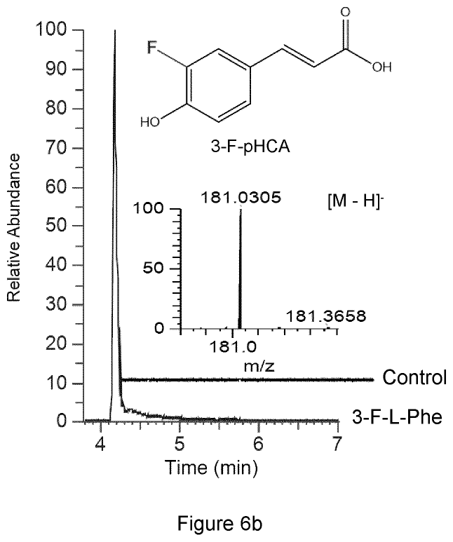

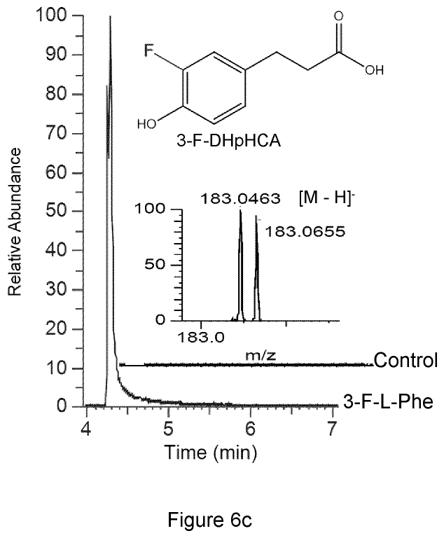

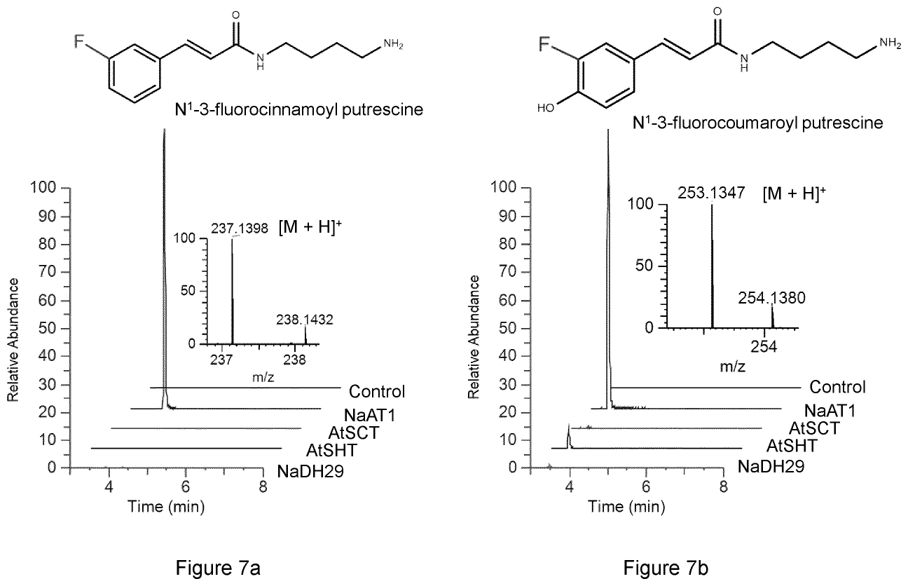

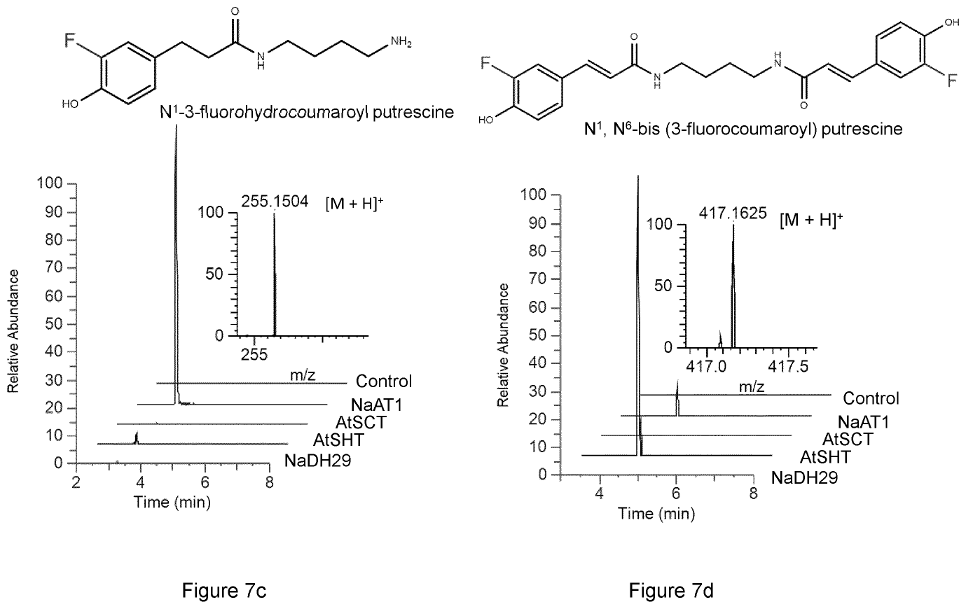

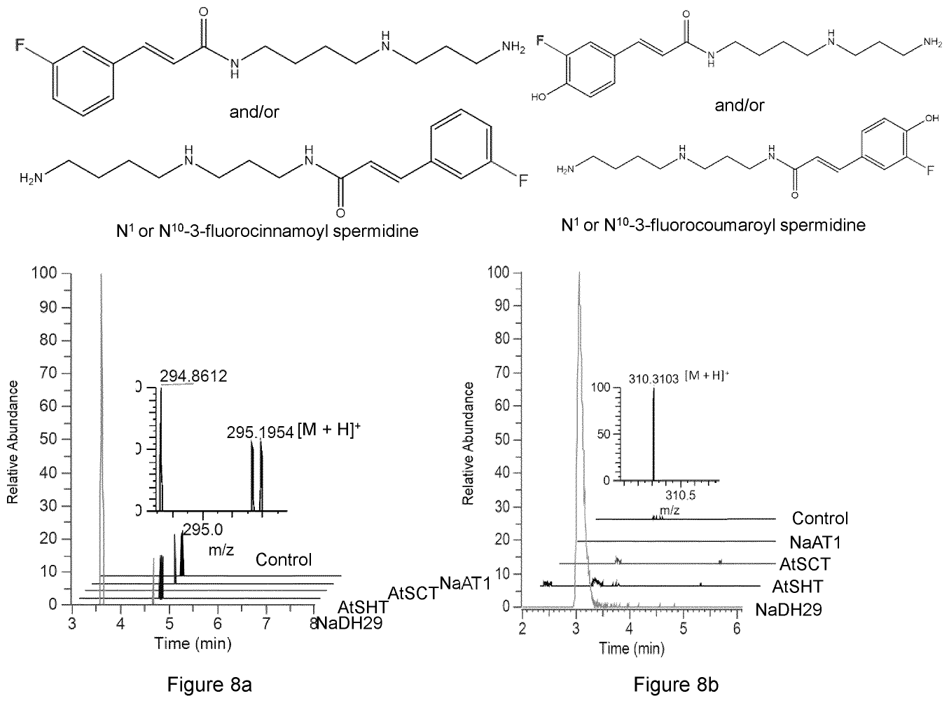

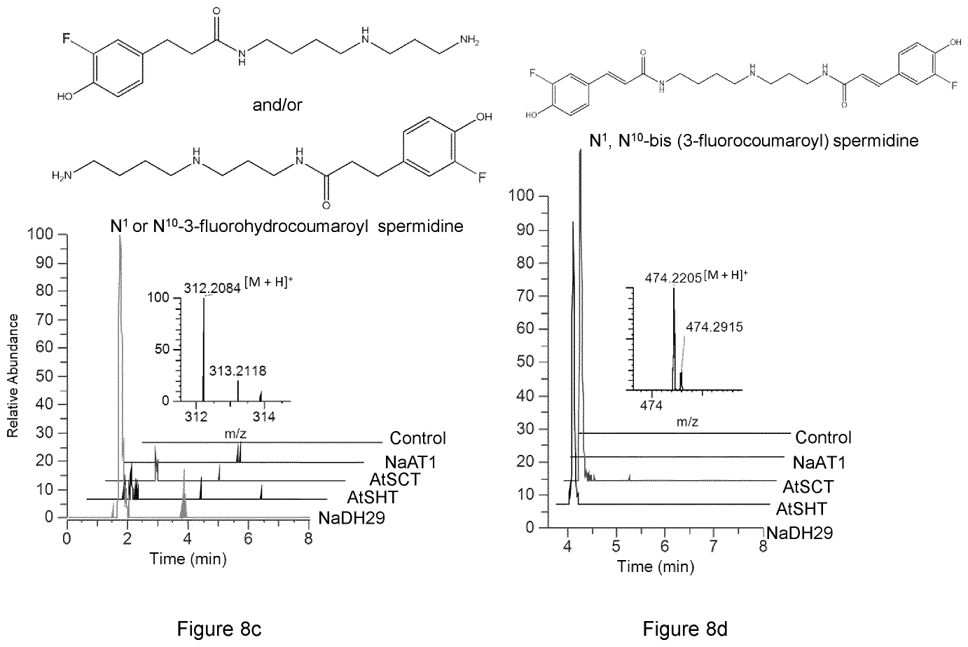

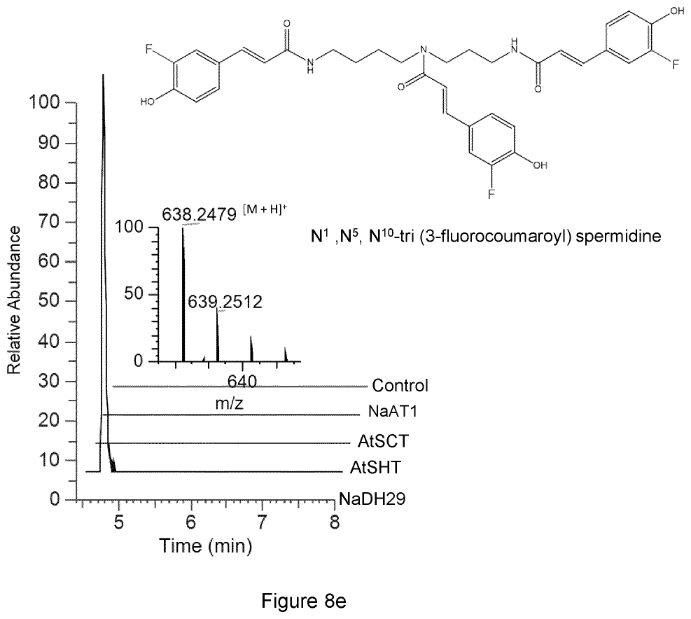

In Example 5, we demonstrated that the de novo production, i.e., with simple sugars as sole carbon source, of naturally existing polyamine-derived phenolamides could be achieved by designing a synthetic consortium comprising a polyamine producing strain and a p-coumaric acid over-producing strain. However, we also noticed that, in addition to their natural counterparts, unnatural polyamine-hydroxycinnamic acid conjugates are actively being investigated for their potentially improved medicinal properties (Mounce et al., 2017; Antoniou et al., 2016). One of the major pharmacophores of interest in this search is halogenated derivatives, such as fluorine substituents, because organofluorine is known to affect absorption, distribution, metabolism, excretion, and toxicity (ADMET) properties of a lead compound (Müller et al., 2007). We set out to establish a biosynthetic approach for the production of this class of fluorine-substituted polyamine-hydroxycinnamic acid conjugates, hypothesizing that the observed promiscuity of 4CLs-NATs systems, i.e., 4-coumarate:CoA ligase plus N-acyltransferase, towards hydroxycinnamic acids, would translate to fluorine-substituted precursors. To get access to fluorine-substituted hydroxycinnamic acids, we used the strain (QL58), which over-produces aromatic chemicals (Liu et al., 2019), and fed this strain a fluorine-substituted aromatic amino acid (3-fluoro-L-phenylalanine). From this, peaks corresponding to the predicted m/z values of 3-fluoro-cinnamic acid ([M−H]⁻=165.0358), 3-fluoro-p-coumaric acid ([M−H]⁻=181.0305) and, fluorine substituted and hydrogenated p-coumaric acid ([M−H]⁻=183.0463) were detected (see FIGS. 6a to 6c), which suggests that the heterologous pathways recruited here for the biosynthesis of aromatics from aromatic amino acid are promiscuous. Subsequent supplementation of co-culture system from Example 5, comprising both polyamine over-producing strains (JQSPD_AA strain co-overexpressing At4CL1 and one of AtSHT, AtSCT, NaDH29 and NaAT1) and aromatic over-producing strain QL58 with 3-fluoro-L-phenylalanine yielded an array of mono-, and di-unnatural fluorine substituted putrescine-hydroxycinnamic acid conjugates (see FIGS. 7a to 7d) as well a list of as mono-, di-, and tri-substituted unnatural fluorine substituted spermidine-hydroxycinnamic acid conjugates (see FIGS. 8a to 8e).

The embodiments described above are to be understood as a few illustrative examples of the present invention. It will be understood by those skilled in the art that various modifications, combinations and changes may be made to the embodiments without departing from the scope of the present invention.

In particular, different part solutions in the different embodiments can be combined in other configurations, where technically possible. The scope of the present invention is, however, defined by the appended claims.

### REFERENCES

Antoniou, A. I., Pepe, D. A., Aiello, D., Siciliano, C. & Athanassopoulos, C. M. Chemoselective protection of glutathione in the preparation of bioconjugates: The case of trypanothione disulfide. J. Org. Chem. 81, 4353-4358, doi:10.1021/acs.joc.6b00300 (2016).
Bienz, S., Detterbeck R., Ensch C., Guggisberg A., Hsusermann U., Meisterhans C., Wendt B., Werner C. & Hesse M. Putrescine, spermidine, spermine, and related polyamine alkaloids. Alkaloids Chem Biol. 58, 83-338 (2002).
Kim, S.-K., Jin, Y.-S., Choi, I.-G., Park, Y.-C. & Seo, J.-H. Enhanced tolerance of Saccharomyces cerevisiae to multiple lignocellulose-derived inhibitors through modulation of spermidine contents. Metabolic Engineering 29, 46-55, doi:http://dx.doi.org/10.1016/j.ymben.2015.02.004 (2015).
Liu, Q. et al. Rewiring carbon metabolism in yeast for high level production of aromatic chemicals. Nat. Commun. 10, 4976, doi:10.1038/s41467-019-12961-5 (2019).
Mans, R., van Rossum, H. M., Wijsman, M., Backx, A., Kuijpers, N. G., van den Broek, M., Daran-Lapujade, P., Pronk, J. T., van Maris, A. J. & Daran, J. M. CRISPR/Cas9: a molecular Swiss army knife for simultaneous introduction of multiple genetic modifications in Saccharomyces cerevisiae. FEMS Yeast Res. 15, fov004-fov004, doi:10.1093/femsyr/fov004 (2015).
Michael, A. J. Polyamines in Eukaryotes, Bacteria, and Archaea. J. Biol. Chem. 291, 14896-14903, doi:10.1074/jbc.R116.734780 (2016).
Mounce, B. C., Olsen, M. E., Vignuzzi, M. & Connor, J. H. Polyamines and their role in virus infection. Microbiol. Mol. Biol. Rev. 81, e00029-00017, doi:10.1128/mmbr.00029-17 (2017).
Müller, K., Faeh, C. & Diederich, F. Fluorine in pharmaceuticals: Looking beyond intuition. Science 317, 1881-1886, doi:10.1126/science.1131943 (2007).
Nielsen, J. Yeast cell factories on the horizon. Science 349, 1050-1051, doi:10.1126/science.aad2081 (2015).
Nielsen, J. Cell factory engineering for improved production of natural products. Natural Product Reports, doi:10.1039/C9NP00005D (2019).
Nielsen, J. & Keasling, Jay D. Engineering Cellular Metabolism. Cell 164, 1185-1197, doi:https://doi.org/10.1016/j.cell.2016.02.004 (2016).
Ober, D. & Hartmann, T. Homospermidine synthase, the first pathway-specific enzyme of pyrrolizidine alkaloid biosynthesis, evolved from deoxyhypusine synthase. Proceedings of the National Academy of Sciences 96, 14777-14782, doi:10.1073/pnas.96.26.14777 (1999).
Rodriguez, A., Kildegaard, K. R., Li, M., Borodina, I. & Nielsen, J. Establishment of a yeast platform strain for production of p-coumaric acid through metabolic engineering of aromatic amino acid biosynthesis. Metabolic Engineering 31, 181-188, doi:https://doi.org/10.1016/j.ymben.2015.08.003 (2015).
Verduyn, C., Postma, E., Scheffers, W. A., Van Dijken, J. P. Effect of benzoic acid on metabolic fluxes in yeasts: A continuous-culture study on the regulation of respiration and alcoholic fermentation. Yeast 8, 501-517, doi:10.1002/yea.320080703 (1992).
Yu, T. Zhou, Y. J., Huang, M., Liu, Q., Pereira, R., David, F., & Nielsen, J. Reprogramming Yeast Metabolism from Alcoholic Fermentation to Lipogenesis. Cell 174, 1549-1558. e1514, doi:https://doi.org/10.1016/j.cell.2018.07.013 (2018).
Zhou, Y. J., Buijs, N. A., Zhu, Z., Qin, J., Siewers, V. & Nielsen, J. Production of fatty acid-derived oleochemicals and biofuels by synthetic yeast cell factories. Nat. Commun 7, 11709, doi:10.1038/ncomms11709, https://www.nature.com/articles/ncomms11709 #supplementary-information (2016).

1. A yeast cell capable of producing at least one polyamine analog, wherein
the yeast cell is capable of producing at least one polyamine;
the yeast cell comprises a 4-coumarate: CoA ligase encoding gene;
the yeast cell comprises at least one polyamine N-acyltransferase gene;
the yeast cell comprises at least one polyamine synthase encoding gene; and
the yeast cell lacks a polyamine oxidase encoding gene or comprises a disrupted polyamine oxidase encoding gene.

2. The yeast cell according to claim 1, wherein the yeast cell is engineered for overexpression of the 4-coumarate:CoA ligase.

3. The yeast cell according to claim 1 or 2, wherein the 4-coumarate:CoA ligase encoding gene is selected from the group consisting of Arabidopsis thaliana At4CL1, At4CL2, At4CL3, At4CL4, At4CL5 and a nucleotide sequence encoding a 4-coumarate:CoA ligase having at least 80% sequence identity with any of 4-coumarate:CoA ligase At4CL1, At4CL2, At4CL3, At4CL4, or At4CL5, preferably Arabidopsis thaliana At4CL1.

4. The yeast cell according to any of the claims 1 to 3, wherein the yeast cell is engineered for overexpression of the at least one polyamine N-acyltransferase.

5. The yeast cell according to any of the claims 1 to 4, wherein the yeast cell comprises the at least one polyamine N-acyltransferase gene selected from the group consisting of a spermidine hydroxycinnamoyl transferase encoding gene, a spermidine coumaroyl-CoA acyltransferase encoding gene and a putrescine hydroxycinnamoyl transferase encoding gene.

6. The yeast cell according to claim 5, wherein the spermidine hydroxycinnamoyl transferase encoding gene is selected from the group consisting of Arabidopsis thaliana AtSHT, Nicotiana attenuata NaDH29 and a nucleotide sequence encoding a spermidine hydroxycinnamoyl transferase having at least 80% sequence identity with spermidine hydroxycinnamoyl transferase AtSHT or spermidine hydroxycinnamoyl transferase NaDH29.

7. The yeast cell according to claim 5 or 6, wherein the spermidine coumaroyl-CoA acyltransferase encoding gene is selected from the group consisting of Arabidopsis thaliana AtSCT and a nucleotide sequence encoding a spermidine coumaroyl-CoA acyltransferase having at least 80% sequence identity with spermidine coumaroyl-CoA acyltransferase AtSCT.

8. The yeast cell according to any of the claims 5 to 7, wherein the putrescine hydroxycinnamoyl transferase encoding gene is selected from the group consisting of Nicotiana attenuata NaAT1 and a nucleotide sequence encoding a putrescine hydroxycinnamoyl transferase having at least 80% sequence identity with putrescine hydroxycinnamoyl transferase NaAT1.

9. The yeast cell according to any of the claims 1 to 8, wherein the yeast cell is capable of producing at least one organic acid selected from the group consisting of an aromatic organic acid, a fatty acid, a halogenated aromatic organic acid, a halogenated fatty acid and a combination thereof.

10. The yeast cell according to any of the claims 1 to 9, wherein the at least one polyamine analog is selected from the group consisting of a polyamine alkaloid, a polyamine-fatty acid conjugate and a combination thereof.

11. The yeast cell according to any of the claims 1 to 10, wherein the at least one polyamine is selected from the group consisting of spermine, thermospermine, sym-homospermidine, 1,3-diaminopropane, putrescine, cadaverine, agmatine, spermidine, sym-norspermidine, norspermine and a combination thereof.

12. The yeast cell according to any of the claims 1 to 11, wherein the yeast cell is engineered for overexpression of the at least one polyamine synthase.

13. The yeast cell according to any of the claims 1 to 12, wherein the polyamine synthase encoding gene is selected from the group consisting of a spermine synthase encoding gene, a thermospermine synthase encoding gene and a homospermidine synthase encoding gene.

14. The yeast cell according to claim 13, wherein the spermine synthase encoding gene is selected from the group consisting of Saccharomyces cerevisiae SPE4, Arabidopsis thaliana AtSPMS and a nucleotide sequence encoding a spermine synthase having at least 80% sequence identity with spermine synthase SPE4 or spermine synthase AtSPMS.

15. The yeast cell according to claim 13 or 14, wherein the thermospermine synthase encoding gene is selected from the group consisting of Arabidopsis thaliana AtACL5 and a nucleotide sequence encoding a thermospermine synthase having at least 80% sequence identity with thermospermine synthase AtACL5.

16. The yeast cell according to any of the claims 13 to 15, wherein the homospermidine synthase encoding gene is selected from the group consisting of Senecio vernalis SvHSS, Blastochloris viridis BvHSS and a nucleotide sequence encoding a homospermidine synthase having at least 80% sequence identity with homospermidine synthase SvHSS or homospermidine synthase BvHSS.

17. The yeast cell according to any of the claims 13 to 16, wherein the yeast cell is a Saccharomyces cerevisiae cell and the polyamine oxidase is FMS1.

18. A method of producing polyamine analogs, the method comprising:
culturing a yeast cell according to any of the claims 1 to 17 in a culture medium and in culture conditions suitable for production of the polyamine analogs by the yeast cell; and
collecting the polyamine analogs from the culture medium and/or from the yeast cell.

19. The method according to claim 18, wherein culturing the yeast cell comprises culturing the yeast cell according to any of the claims 1 to 17 in the culture medium comprising at least one organic acid selected from the group consisting of an aromatic organic acid, a fatty acid, a halogenated aromatic organic acid, a halogenated fatty acid and a combination thereof.

20. The method according to claim 19, further comprising adding the at least one organic acid to the culture medium.

21. The method according to claim 19 or 20, wherein culturing the yeast cell comprises co-culturing the yeast cell according to any of the claims 1 to 17 in the culture medium with a microorganism capable of producing the at least one organic acid and releasing the at least one organic acid into the culture medium.


SEQUENCE LISTING (266 sequences)

SEQ ID NO: 1 - protein, 561 aa, Arabidopsis thaliana
MAPQEQAVSQVMEKQSNNNNSDVIFRSKLPDIYIPNHLSLHDYIFQNISEFATKPCLINGPTGHVYTYSDVHVISRQIAANFHKLGVNQNDVVMLLLPNCPEFVLSFLAASFRGATATAANPFFTPAEIAKQAKASNTKLIITEARYVDKIKPLQNDDGVVIVCIDDNESVPIPEGCLRFTELTQSTTEASEVIDSVEISPDDVVALPYSSGTTGLPKGVMLTHKGLVTSVAQQVDGENPNLYFHSDDVILCVLPMFHIYALNSIMLCGLRVGAAILIMPKFEINLLLELIQRCKVTVAPMVPPIVLAIAKSSETEKYDLSSIRVVKSGAAPLGKELEDAVNAKFPNAKLGQGYGMTEAGPVLAMSLGFAKEPFPVKSGACGTVVRNAEMKIVDPDTGDSLSRNQPGEICIRGHQIMKGYLNNPAATAETIDKDGWLHTGDIGLIDDDDELFIVDRLKELIKYKGFQVAPAELEALLIGHPDITDVAVVAMKEEAAGEVPVAFVVKSKDSELSEDDVKQFVSKQVVFYKRINKVFFTESIPKAPSGKILRKDLRAKLANGL

SEQ ID NO: 2 - DNA, 1686 bp, Arabidopsis thaliana
atggccccgcaggaacaagcggttagtcaagtaatggaaaagcagtcaaacaataataatagtgacgttatatttcgtagtaaattgccggacatatacattccgaaccaccttagcttacacgactacatctttcaaaacatttctgagtttgccacaaagccatgccttattaacggtcccacaggtcacgtctatacctacagcgacgtacacgtcatcagccgtcaaatagctgctaatttccataaacttggtgttaatcaaaatgatgtcgtgatgctattgctgccgaattgtcctgagtttgttttgtcatttttagccgcatcatttagaggcgccactgctaccgcggcaaatcctttcttcaccccagcggagatagcgaagcaagctaaggccagtaatacaaaattaattatcaccgaggcgcgttatgttgataaaatcaaaccacttcaaaatgacgacggagtggtcatagtgtgcatagatgataatgaaagcgtgccgattcccgaaggatgcttgcgtttcaccgagctaactcagagtacaacagaggcaagcgaggtgatagactccgtggagattagtccggatgatgttgtagctctgccttatagctccgggaccaccggactaccaaagggggtaatgctaacacacaaaggtctggtgaccagcgtcgcgcagcaagtggatggggaaaacccgaacctttatttccatagcgacgatgttatactatgtgttctaccgatgttccatatttatgctctgaactccataatgttgtgcgggctgcgtgtgggagcggcgattctaataatgccaaaattcgagattaatttacttttagaattaatacaaagatgtaaggtgacggtcgcacctatggtcccccctatagtactggctatagcgaagtcaagtgagacagaaaaatacgatttgagcagcataagggttgtcaagtcaggggctgctccattaggtaaagagttggaagacgcggtcaacgccaaattccctaacgcaaagctaggccaaggatatggcatgaccgaagctggaccagtattagccatgagtctgggcttcgccaaagagccatttccggtcaaatccggggcgtgcggaaccgtcgtcagaaacgctgagatgaaaatcgtcgatcccgacacaggtgatagcctatcaaggaatcaaccaggggagatatgtatcagaggacatcagatcatgaagggttacctgaacaaccccgcagcaacagcggagacgatcgataaagacggatggcttcacacaggagatatcgggctaattgatgacgatgatgagcttttcatcgtagaccgtctaaaagagcttatcaagtataaaggtttccaggtcgcgcctgctgagttagaggccttgctgattggacatccggatataaccgatgtcgcagtagtggcaatgaaggaggaggcagctggcgaagtccctgttgcatttgttgtgaaatcaaaggacagcgagttaagtgaagacgacgtcaagcaattcgtatccaaacaggtagtattctacaaaaggataaacaaggttttttttactgaaagcattccaaaagccccatccggcaagatactgcgtaaggacttaagggcgaagctggccaatggtctttaa

SEQ ID NO: 3 - protein, 451 aa, Arabidopsis thaliana
MAPITFRKSYTIVPAEPTWSGRFPLAEWDQVGTITHIPTLYFYDKPSESFQGNVVEILKTSLSRVLVHFYPMAGRLRWLPRGRFELNCNAEGVEFIEAESEGKLSDFKDFSPTPEFENLMPQVNYKNPIETIPLFLAQVTKFKCGGISLSVNVSHAIVDGQSALHLISEWGRLARGEPLETVPFLDRKILWAGEPLPPFVSPPKFDHKEFDQPPFLIGETDNVEERKKKTIVVMLPLSTSQLQKLRSKANGSKHSDPAKGFTRYETVTGHVWRCACKARGHSPEQPTALGICIDTRSRMEPPLPRGYFGNATLDVVAASTSGELISNELGFAASLISKAIKNVTNEYVMIGIEYLKNQKDLKKFQDLHALGSTEGPFYGNPNLGVVSWLTLPMYGLDFGWGKEFYTGPGTHDFDGDSLILPDQNEDGSVILATCLQVAHMEAFKKHFYEDI

SEQ ID NO: 4 - DNA, 1356 bp, Arabidopsis thaliana
atggctccgattactttcagaaaatcttacactatagtccccgctgagccaacgtggagcggaaggtttcctcttgctgagtgggaccaggtggggacgataacacatatcccaaccctatatttctatgacaagccatcagagtcctttcaggggaatgtcgtggagattctaaagactagcctttccagggtgttggtacacttttacccgatggcaggaaggctaagatggcttcccaggggtcgtttcgagttgaactgtaacgcggaaggagtcgaattcatcgaagcggagtcagaaggaaaactttctgacttcaaggacttttccccgacgccagagttcgagaacctaatgcctcaagttaattacaagaacccgatcgagactattcctctatttttagcgcaagtgactaagttcaagtgtggaggtatatcactgtctgtgaacgtctctcacgcaattgttgacggacaaagtgctttgcatttaatatcagagtgggggcgtctggcaaggggcgagccgctggagacggttccattcttggatcgtaaaatactttgggccggagaaccgctgcccccttttgtttccccacctaagtttgaccacaaagagtttgaccaacctccgtttttgataggagagaccgacaatgtggaagaaagaaaaaaaaagacaatcgttgtcatgttaccgctttccacctctcaactacagaaattgcgttcaaaagcgaacggatcaaaacactccgatcctgcaaagggtttcaccaggtatgagaccgtgaccggccacgtctggcgttgcgcatgtaaggccagaggacacagccccgagcaacccaccgccttggggatatgtatcgacacccgttctaggatggaaccgcccctgccaagaggttatttcggaaacgcgaccttagatgttgtagcggcctctactagcggtgagcttattagcaatgaattaggattcgctgcgagtctaatttctaaagccatcaaaaatgtcaccaatgagtatgtaatgatcgggatagaataccttaagaatcaaaaggatttgaaaaaatttcaggaccttcacgccttgggcagtacggaagggcccttctacggcaacccgaatttgggcgtcgtcagttggctgacgctgccaatgtatgggctggactttggttgggggaaggaattttatactggcccaggcacgcacgattttgatggcgactctcttatcttgccggatcagaacgaagatggaagtgtcatccttgccacgtgtcttcaagtcgcgcatatggaggccttcaaaaaacacttctatgaggatatctaa

SEQ ID NO: 5 - protein, 449 aa, Nicotiana attenuata
MGFLCANLKNSLAVEIMSKKLVKPSSPTPTHLQSYKLSFFDQLAIRMHVPIVLIYHNLNNSITNELLEESLSKTLTHVYPSAGRINKDRRVVDCLDQGVEFIIAKVNCQLEDFLEQARKDIDLANHFWPQGIKDVDDNYDFAITPLVFVQVTRFECGGLALSVAAEHIAIDGFTNMKFIYEWAKVCRLGIPTSTTTDIFNYDLGDIFPARDTSRILKPLASLAIPKDTITYVAKRFVFNEASISKLRNKIASGVLSFKPSRVEIVTALLWRALIRASQAKNGRLRPSLMSFPVNLRGKASLPKLSDTFGNFAVEVPVVFTPNETKMELHNLIALIRDATDKTMVSSAKASNDELVSMAANLYNMTQEWEANEEVDEFTCSSLCRFPMKEADFGLGKPCWMTFGLRQSQVFWLYDADFGSSIAAQVDLNESLMHYFERDQDLNTFTILNN

SEQ ID NO: 6 - DNA, 1350 bp, Nicotiana attenuata
atgggattcttatgtgcgaacttgaaaaattctcttgccgtggaaataatgagtaagaaactggtgaaaccatcctctcccacacccacacacttacaaagctataaactttccttttttgatcaactagccatcaggatgcatgtgcctatcgttctaatatatcataatctaaacaattctattaccaacgaactgcttgaggagagcctgtctaaaaccctgacccatgtctatccatctgccggaagaatcaacaaggacaggcgtgtggttgactgcttggaccagggcgtcgaattcattatagctaaggtaaactgccaactagaagattttctagaacaggccaggaaagatattgacttagctaatcacttttggcctcaggggataaaagatgtcgatgataattacgattttgcgattactccccttgtgtttgtacaggtcactaggttcgagtgcgggggattggctttgtctgtcgcagccgagcacatagctattgatggattcactaatatgaagtttatatatgagtgggctaaggtatgcagattaggtatccctacctcaacaacgactgatatcttcaactacgacttaggagatatctttcctgcccgtgataccagcaggatattgaaaccccttgcgtcacttgcaatacccaaggatacaattacttacgtggccaagaggtttgtgttcaacgaggcaagcatctcaaagcttcgtaacaaaatcgctagcggtgtgctttcttttaagccgtcacgtgtggaaatagttactgctttattgtggagggcacttatcagagccagccaagcgaaaaatggaagattacgtccgagccttatgtccttcccggtgaacctgaggggcaaagcttcactacctaagctttccgacacttttggcaattttgctgttgaggttcccgtagtttttacacctaatgaaaccaaaatggagttacataacctgatcgcgttaatacgtgacgcaacggataagacgatggtgtcaagtgccaaagcatctaacgacgagctggtttctatggctgcaaacttgtacaacatgactcaagaatgggaagcaaacgaagaagtcgacgaatttacctgtagtagcctatgccgtttccctatgaaagaagcagacttcggtctgggtaagccgtgttggatgacattcgggttgagacagtcacaggtcttctggttatacgacgcggacttcgggagcagtatagctgcacaggtcgatctgaatgaaagcctaatgcactattttgagagggaccaggatttgaataccttcaccatcctgaacaactaa

SEQ ID NO: 7 - protein, 461 aa, Arabidopsis thaliana
MANQRKPILPLLLEKKPVELVKPSKHTHCETLSLSTLDNDPFNEVMYATIYVFKANGKNLDDPVSLLRKALSELLVHYYPLSGKLMRSESNGKLQLVYLGEGVPFEVATSTLDLSSLNYIENLDDQVALRLVPEIEIDYESNVCYHPLALQVTKFACGGFTIGTALTHAVCDGYGVAQIIHALTELAAGKTEPSVKSVWQRERLVGKIDNKPGKVPGSHIDGFLATSAYLPTTDVVTETINIRAGDIKRLKDSMMKECEYLKESFTTYEVLSSYIWKLRSRALKLNPDGITVLGVAVGIRHVLDPPLPKGYYGNAYIDVYVELTVRELEESSISNIANRVKKAKKTAYEKGYIEEELKNTERLMRDDSMFEGVSDGLFFLTDWRNIGWFGSMDFGWNEPVNLRPLTQRESTVHVGMILKPSKSDPSMEGGVKVIMKLPRDAMVEFKREMATMKKLYFGDTN

SEQ ID NO: 8 - DNA, 1386 bp, Arabidopsis thaliana
atggcaaatcaaagaaaaccgatattaccgctactacttgaaaagaagccagtagagttagtgaaaccctccaagcatactcactgcgagacacttagtttatccacgctagataatgatccctttaatgaagtaatgtacgccacgatatacgtgttcaaagcgaacggcaagaatcttgacgacccagtatcccttcttaggaaagcgctatctgaacttcttgtgcactattacccacttagtggtaaattgatgcgttcagaaagtaatgggaagctacaacttgtttaccttggggaaggagtaccgttcgaggtcgcaacctctacgttggacttatcttctctgaactatatcgagaatttggatgaccaggtcgcgttaagacttgttcccgaaattgaaattgattatgaatctaacgtatgttaccatccattagcattgcaggttactaagttcgcctgtggaggatttactatcgggaccgcacttacacacgctgtgtgtgacggctatggggtcgcccagattatacacgctttaactgaacttgctgcgggaaaaactgagccgagcgtcaaatccgtttggcaacgtgaaagacttgtggggaaaattgacaataaacctggtaaggtaccaggaagtcatatcgacggatttctagccacaagcgcgtacctaccgacaacagatgtagtcacggagactataaatatcagagcgggagacataaaaaggttgaaggacagcatgatgaaagaatgcgagtatctgaaggaatccttcaccacgtatgaagtcttaagttcctacatatggaaactaagaagccgtgcgttaaagctaaaccccgatggcattactgttcttggcgtcgccgtcggcattcgtcacgtactggatccgccattacctaagggctattacggaaatgcctatattgacgtgtacgttgagctaacggttagagaacttgaagagtcaagtatatccaatatagcgaatcgtgtcaagaaagccaagaaaaccgcctacgaaaaaggatacatagaagaggaattgaaaaacaccgaaaggttgatgagggatgattctatgtttgaaggggtgagtgatgggttgttcttcctaaccgattggcgtaatatcggttggttcgggtcaatggattttggttggaatgagcctgtaaatcttcgtccgttaacccagagagaaagcactgtccatgtcggtatgatcttaaagccctccaaatcagacccgtctatggagggaggtgtaaaagttattatgaagcttcccagggacgcgatggtggagttcaagcgtgagatggcaactatgaagaagttgtattttggcgacactaattaa

SEQ ID NO: 9 - protein, 438 aa, Nicotiana attenuata
MNVKIESSRIIKPFYEGTPPSTNTHISFNVFDNVTYDALMALIYAYRPPTPPTSTIEMGLRKTLAVYREWAGRIGRDENGNRVVFLNDEGVRFIEASVNATLDEVLPLKPSPSLLKLHPGMKDVVELIQVQVTRFTCGSVMVGFTGHHMIADGHAASNFFVAWGQACRGVEITPLPLHDRAIFHPRNPPLIEFNHVGAEFMSKSLNKKEFIKLENTEKNIIVHKVHFTLEFLGKLKANASFMNGKTKTYSTFESLVAHLWRVITKARELDGSQNTQIRISVDGRRRVVPRVADEFFGNIVLWAFPTSKVRDLVNEPLHYATKIIHDAITKVDDKYFKSFIDFANHKVTEDLIPTADMKKDTLCPNLEVDSWLRFPFYDLDFGTGCPFVFMPSYYPTEGMMFLVPSFIGDGSIDAFIPLYQDNSPTFKKICYSLDLKAK

SEQ ID NO: 10 - DNA, 1317 bp, Nicotiana attenuata
atgaacgttaagatcgaatcttcaagaattattaagccattttatgaaggtactccaccatctactaatacacatatctcttttaatgttttcgataacgttacatacgatgctttgatggcattaatctatgcttacagaccaccaactccaccaacttctacaatcgaaatgggtttaagaaagacattggctgtttacagagaatgggcaggtagaattggtagagatgaaaacggtaacagagttgttttcttgaatgatgaaggtgttagattcattgaagcttcagttaatgcaacattggatgaagttttgccattgaagccatctccatcattgttgaagttgcatcctggtatgaaggatgttgttgaattgatccaagttcaagttactagattcacttgtggttctgttatggttggttttactggtcatcatatgattgctgatggtcatgctgcatcaaatttctttgttgcttggggtcaagcatgtagaggtgttgaaattacaccattgccattacatgatagagctatcttccatccaagaaacccaccattgatcgagtttaatcatgttggtgcagagtttatgtctaagtcattgaataagaaagagtttattaaattggaaaatactgaaaagaatattattgttcataaagttcattttacattggaatttttgggtaaattgaaggctaacgcatcttttatgaacggtaaaactaagacatactctactttcgaatcattggttgctcatttgtggagagttattactaaggcaagagaattggatggttctcaaaacacacaaatcagaatctcagttgatggtagaagaagagttgttccaagagttgctgatgaatttttcggtaacatcgttttgtgggcatttccaacatctaaagttagagatttggttaacgaaccattgcattacgctactaagatcatccatgatgcaattacaaaagttgatgataagtacttcaagtcttttattgattttgctaatcataaagttactgaagatttgattccaacagcagatatgaagaaagatactttgtgtccaaatttggaagttgattcttggttgagattcccattctacgatttggatttcggtactggttgtccattcgtttttatgccatcatactacccaacagagggtatgatgttcttggttccatcttttattggtgacggttcaatcgatgcttttattccattgtaccaagataactctccaacttttaagaaaatttgttactcattggatttgaaagcaaaataa

SEQ ID NO: 11 - protein, 300 aa, Saccharomyces cerevisiae
MVNNSQHSYIKDGWFREINDKSFPGQAFTMTVDSILYEARSEFQDILIFRNKVYGTVLVLDGIVQCTEFDEFAYQEMITHIAMFAHSNPKRVLIIGGGDGGVLREVAKHSCVEDITMVEIDSSVIELSRKFLPTLSNGAFDDERLDLKLCDGFKFLQDIGASDVHKKFDVIITDSSDPEGPAEAFFQERYFELLKDALNPNGVVIMQSSENFWLNLKYLHDLKNTAKKVFPNTEYCYTMVPTYTSGQLGLIVCSNNANIPLNIPQRKISEQEQGKLKYYNPQIHSSAFVLPTWADKVINE

SEQ ID NO: 12 - DNA, 904 bp, Saccharomyces cerevisiae
atggttaacaactctcaacatccatacatcaaggatggttggttcagagaaattaatgataagtcattcccaggtcaagcttttactatgacagttgattctatcttgtacgaagcaagatcagaatttcaagatatcttgatttttagaaataaggtttacggtactgttttggttttggatggtatcgttcaatgtacagaatttgatgaatttgcttaccaagaaatgatcactcatatcgctatgttcgcacattctaacccaaaaagagttttgatcattggtggtggtgacggtggtgttttgagagaagttgcaaagcattcatgtgttgaagatatcactatggttgaaatcgattcttcagttattgaattgtctagaaagttcttaccaacattgtcaaatggtgctttcgatgatgaaagattggatttgaagttgtgtgatggttttaaattcttgcaagatatcggtgcatctgatgttcataagaaattcgatgttatcatcactgattcttcagatccagaaggtccagctgaagctttctttcaagaaagatacttcgaattgttgaaggatgctttgaacccaaacggtgttgttattatgcaatcttcagaaaacttctggttgaatttgaagtatttgcatgatttgaaaaatacagctaagaaagttttcccaaacactgaatactgttacacaatggttccaacttacacatctggtcaattaggtttgatcgtttgttcaaacaacgctaacatcccattgaacatcccacaaagaaaaatttctgaacaagaacagggtaaattgaagtactacaacccacaaatccattcttcagcttttgttttgccaacatgggcagataaagttattaatgaataag

SEQ ID NO: 13 - protein, 359 aa, Arabidopsis thaliana
MEGDVGKGLVCQNTMDGKASNGNGLEKTVPSCCLKAMACVPEDDAKCHSTVVSGWFSEPHPRSGKKGGKAVYFNNPMWPGEAHSLKVEKVLFKDKSDFQEVLVFESATYGKVLVLDGIVQLTEKDECAYQEMIAHLPLCSISSPKNVLVVGGGDGGVLREISRHSSVEVIDICEIDKMVIDVSKKFFPELAVGFDDPRVQLHIGDAAEFLRKSPEGKYDAIIVDSSDPVGPALALVEKPFFETLARALKPGGVLCNMAESMWLHTHLIEDMISICRQTFKSVHYAWSSVPTYPSGVIGFVLCSTEGPAVDFKNPINPIEKLDGAMTHKRELKFYNSDMHRAAFALPTFLRREVASLLAS

SEQ ID NO: 14 - DNA, 1080 bp, Arabidopsis thaliana
atggaaggtgacgttggtaaaggtttggtttgtcaaaatactatggatggtaaagcttcaaatggtaatggtttggaaaagactgttccatcttgttgtttaaaagctatggcatgtgttccagaagatgatgcaaaatgtcattctactgttgtttcaggttggttttctgaaccacatccaagatcaggtaaaaagggtggtaaagctgtttacttcaacaacccaatgtggccaggtgaagcacattctttgaaggttgaaaaggttttgtttaaagacaagtcagattttcaagaagttttggttttcgaatctgctacttacggtaaagttttggttttggatggtatcgttcaattgacagaaaaggatgaatgtgcttaccaagaaatgattgcacatttgccattgtgttctatctcttcacctaaaaatgttttggttgttggtggtggtgacggtggtgttttgagagaaatctcaagacattcttcagttgaagttattgatatctgtgaaatcgataagatggttattgatgtttctaagaaatttttcccagaattagctgttggttttgatgatccaagagttcaattgcatattggtgacgctgcagaatttttgagaaagtcaccagagggtaaatacgatgcaatcatcgttgattcttcagatccagttggtccagctttggcattggttgaaaagccatttttcgaaacattggctagagcattaaaaccaggtggtgttttgtgtaatatggctgaatctatgtggttgcatactcatttgatcgaagatatgatctcaatctgtagacaaacttttaaatctgttcattacgcatggtcttcagttccaacttacccatcaggtgttattggtttcgttttgtgttctacagaaggtccagctgttgatttcaagaacccaattaatccaatcgaaaaattggatggtgcaatgactcataagagagaattgaagttctacaattctgatatgcatagagctgcatttgctttgccaacatttttaagaagagaagttgcttcattgttagcatcttaa

SEQ ID NO: 15 - protein, 339 aa, Arabidopsis thaliana
MGEAVEVMFGNGFPEIHKATSPTQTLHSNQQDCHWYEETIDDDLKWSFALNSVLHQGTSEYQDIALLDTKRFGKVLVIDGKMQSAERDEFIYHECLIHPALLFHPNPKTVFIMGGGEGSAAREILKHTTIEKVVMCDIDQEVVDFCRRFLTVNSDAFCNKKLELVIKDAKAELEKREEKFDIIVGDLADPVEGGPCYQLYTKSFYQNILKPKLSPNGIFVTQAGPAGIFTHKEVFTSIYNTMKQVFKYVKAYTAHVPSFADTWGWVMASDHEFDVEVDEMDRRIEERVNGELMYLNAPSFVSAATLNKTISLALEKETEVYSEENARFIHGHGVAYRHI

SEQ ID NO: 16 - DNA, 1020 bp, Arabidopsis thaliana
atgggtgaagcagtagaagtaatgttcggtaacggtttcccagaaatcttaaaagccacaagtccaactcaaaccttgcactccaatcaacaagattgtcattggtacgaagaaactatcgatgatgatttgaagtggtctttcgctttaaattctgttttgcatcaaggtacttctgaataccaagatatcgcattgttggatacaaagagattcggtaaagttttggttattgatggtaaaatgcaatcagctgaaagagatgagtttatatatcatgaatgtttgatccatccagcattgttgttccatccaaacccaaagactgtttttattatgggtggtggtgaaggttctgctgcaagagaaatcttgaagcatactacaatcgaaaaagttgttatgtgtgatatcgatcaagaagttgttgatttctgtagaagatttttgacagttaattcagatgctttctgtaataagaaattggaattagttattaaagatgctaaggcagaattggaaaagagagaagaaaagttcgatattattgttggtgacttggctgatccagttgaaggtggtccatgttatcaattgtacactaagtctttctaccaaaacattttgaaaccaaaattatcaccaaatggtatttttgttactcaagctggtccagcaggtatttttacacataaagaagtttttacttctatctataacacaatgaagcaagtttttaaatacgttaaagcttacactgcacatgttccatcttttgctgatacatggggttgggttatggcatcagatcatgaatttgatgttgaagttgatgaaatggatagaagaatcgaagaaagagttaacggtgaattgatgtacttaaatgctccatcttttgtttcagctgcaactttgaataagacaatctcattggcattggaaaaggaaacagaagtttactccgaagaaaatgctagattcatccacggtcacggtgttgcctacagacatatctaa

SEQ ID NO: 17 - protein, 370 aa, Senecio vernalis
MAESNKEAIDSARSNVFKESESLEGTCAKIGGYDFNNGIDHSKLLKSMVSTGFQASNLGDAMIITNQMLDWRLSHDEVPENCSEEEKKNRESVKCKIFLGFTSNLISSGVRETICYLTQHRMVDVLVTTTGGIEEDFIKCLASTYKGKFSLPGADLRSKGLNRIGNLIVPNDNYIKFEDWIIPIFDQMLIEQKTQNVLWTPSRMIARLGKEINNETSYLYWAYKNNIPVFCPSITDGSIGDMLYFHSVSNPGPGLVVDIVQDVIAMDNEAVHASPQKTGIIILGGGLPKHHICNANMMRNGADFAVFINTAQEYDGSDSGARPDEAVSWGKISSTGKAVKVHCDATIAFPLLVAETFAVKKEKASKVNGF

SEQ ID NO: 18 - DNA, 1113 bp, Senecio vernalis
atggctgaatctaataaggaagctatcgattctgcaagatcaaacgtttttaaagaatctgaatcattagaaggtacatgtgcaaagatcggtggttacgatttcaacaacggtatcgatcattcaaagttgttgaagtctatggtttcaacaggtttccaagcttctaatttgggtgacgcaatgatcatcactaaccaaatgttggattggagattatctcatgatgaagttccagaaaactgttcagaagaagaaaagaaaaatagagaatctgttaagtgtaagattttcttgggttttacatcaaatttgatctcttcaggtgttagagaaacaatctgttatttgactcaacatagaatggttgatgttttggttactacaactggtggtatcgaagaagatttcatcaagtgtttagcttctacttacaagggtaaattttcattgccaggtgcagatttgagatctaagggtttgaacagaattggtaatttgatcgttccaaacgataactacatcaagttcgaagattggattattccaatttttgatcaaatgttaattgaacaaaagactcaaaatgttttgtggactccatcaagaatgattgctagattgggtaaagaaattaataacgaaacatcttatttgtactgggcatacaagaacaacatcccagttttctgtccatctattactgatggttcaattggtgacatgttgtacttccattctgtttcaaacccaggtccaggtttggttgttgatatcgttcaagatgttatcgctatggataatgaagctgttcatgcatctccacaaaagactggtatcatcatcttgggtggtggtttaccaaagcatcatatctgtaacgctaacatgatgagaaacggtgctgatttcgcagtttttattaacacagcacaagaatacgatggttctgattcaggtgctagaccagatgaagcagtttcatggggtaaaatctcttcaactggtaaagctgttaaagttcattgtgatgctacaattgcatttccattgttagttgctgaaactttcgcagttaagaaagaaaaggcttctaaagttaatggtttttaa

SEQ ID NO: 19 - protein, 280 aa, Blastochloris viridis
MTDWPVYHRIDGPIVMIGFGSIGRGTLPLIERHFAFDRSKLVVIDPSDEARKLAEARGVRFIQQAVTRDNYRDLLVPLLTAGPGQGFCVNLSVDTSSLDIMELARENGALYIDTVVEPWLGFYFDPDLKPEARSNYALRETVLAARRNKPGGTTAVSCCGANPGMVSWFVKQALVNLAADLGVTREEPTTREEWARLAMDLGVKGIHIAERDTQRANFPKPFDVFVNTWSVEGFVSEGLQPAELGWGTFERWMPDNARGHDSGCGAGIYLLQPGANTRVR

SEQ ID NO: 20 - DNA, 1434 bp, Blastochloris viridis
atgacagattggccagtttaccatagaatcgatggtccaatcgttatgattggttttggttctattggtagaggtactttgccattgatcgaaagacatttcgcattcgatagatctaagttggttgttattgatccatcagatgaagctagaaaattggctgaagcaagaggtgttagattcattcaacaagcagttacaagagataactacagagatttgttggttccattgttaactgctggtccaggtcaaggtttctgtgttaatttgtctgttgatacatcttcattggatatcatggaattggctagagaaaatggtgcattgtatattgatactgttgttgaaccatggttgggtttctacttcgatccagatttgaagccagaagctagatcaaactacgcattgagagaaacagttttagctgcaagaagaaataagccaggtggtactacagctgtttcttgttgtggtgcaaatcctggtatggtttcatggttcgttaagcaagctttggttaatttggctgcagatttgggtgttactagagaagaaccaactacaagagaagaatgggctagattggcaatggatttgggtgttaagggtattcatatcgctgaaagagatacacaaagagcaaacttcccaaagccattcgatgttttcgttaacacttggtctgttgaaggttttgtttcagaaggtttgcaaccagctgaattaggttggggtacatttgaaagatggatgccagataatgctagaggtcatgattctggttgtggtgcaggtatatatttgttacaaccaggtgctaatactagagttagatcatggactccaacagctactgcacaatacggtttcttggttacacataacgaatctatctcaatcgcagatttcttgactgttagagatgctgcaggtcaagctgtttatagaccaacatgtcattatgcttaccatccatgtaacgatgcagttttgtctttgcatgaaatgtttggttctggtaaaagacaatcagattggttgatcttggatgaaactgaaatcgttgatggtatcgatgaattgggtgttttgttgtacggtcatggtaaaaatgcttattggtacggttctcaattgtcaatcgaagaaacaagaagaattgctccagatcaaaatgcaactggtttgcaagtttcttcagctgttttagctggtatggtttgggctttggaaaatccaaaagctggtattgttgaagcagatgatttggattacagaagatgtttggaagttcaaacaccatatttgggtccagttgttggtgtttacacagattggactccattggcaggtagaccaggtttatttccagaagatattgatgcttcagatccatggcaattcagaaatgttttggttagagattaa

SEQ ID NO: 21 - DNA, 1365 bp, Saccharomyces cerevisiae
atgtcagagccagaatttcaacaagcttacgaagaagttgtctcctctttggaagactctactcttttcgaacaacacccagaatacagaaaggttttgccaattgtttctgttccagaaagaatcatacaattcagagtcacctgggaaaatgacaagggtgaacaagaagttgctcaaggttacagagtgcaatataactccgccaagggtccatacaagggtggtctacgtttccatccttccgtgaacttgtctatcttgaaattcttgggtttcgaacaaatcttcaagaactccttgaccggcctagacatgggtggtggtaaaggtggtctatgtgtggacttgaagggaagatctaataacgaaatcagaagaatctgttatgctttcatgagagaattgagcagacacattggtcaagacactgacgtgccagctggtgatatcggtgttggtggtcgtgaaattggttacctgttcggtgcttacagatcatacaagaactcctgggaaggtgtcttaaccggtaagggtttgaactggggtggttctttgatcagaccagaagccactggttacggtttagtttactatactcaagctatgatcgactatgccacaaacggtaaggaatctttcgaaggtaagcgcgtcaccatctctggtagtggtaacgttgctcaatacgctgccttgaaggttattgagctaggtggtactgtcgtttccctatctgactccaagggttgtatcatctctgaaactggtatcacctccgaacaagtcgctgatatttccagtgctaaggtcaacttcaagtccttggaacaaatcgtcaacgaatactctactttctccgaaaacaaagtgcaatacattgctggtgctcgtccatggacccacgtccaaaaggtcgacattgctttgccatgtgccacccaaaatgaagtcagcggtgaagaagccaaggccttggttgctcaaggtgtcaagtttattgccgaaggttccaacatgggttccactccagaagctattgccgtctttgaaactgctcgttccaccgccactggaccaagcgaagctgtttggtacggtccaccaaaggctgctaacttgggtggtgttgctgtttctggtttagaaatggcacaaaactctcaaagaatcacatggactagcgaaagagttgaccaagagttgaagagaattatgatcaactgtttcaatgaatgtatcgactatgccaagaagtacactaaggacggtaaggtcttgccatctttggtcaaaggtgctaatatcgcaagtttcatcaaggtctctgatgctatgtttgaccaaggtgatgtattttaa

SEQ ID NO: 22 - DNA, 2709 bp, Saccharomyces cerevisiae
atggagcaaatcaattcgaacagtagaaaaaagaagcaacaattggaagtattcaaatattttgcaagtgtccttacaaaagaggacaagcctattagtatcagtaatggtatgttagatatgccgacagtgaactccagtaaactcacagcaggaaatgggaaacctgacacggagaagcttacaggagaactaattttaacatacgacgatttcattgaactgatatctagctcaaagactatttattcgaagtttacggaccattcgttcaatttgaaccagatacccaagaacgttttcgggtgtattttcttcgctattgatgaacaaaacaagggatatctgacgcttaatgattggttttattttaataatttattagaatatgataattatcatctcattattctatatgagttctttaggaaatttgatgtagagaatttgaaggcaaaacaaaaaaaagagcttggtagttcgtcgtttaatttaaaggctgcagatgatcgaattaagtcaattaattatggtaacagatttctaagctttgatgatcttcttttgaatctgaaccaattcaaagatactatccgcctgttgcacgaatctattgatgataattttgttaaagataacaaattactacttgattggaatgactttcgatttctgaaattttacaaatgttatcatgaaaatgaagagtatttgagtttaaactctctggtcacgattttacaaaatgatcttaagaatgaaaaaatatttataggttttgataggttggcacagatggactcacaagggcatcgtttagccctaagcaaaaatcaactcacctatcttctaaggttattttactctcacagggtgtctgcagatatattttcctccttgaatctatcaaacaccgaattactaaaagcggacaataattccattccgtacaatgtattcaaggatatattttatttatttcaaaattttgacctactgaaccaaatatttcacaagtatgttactgaaaataatttgaatgagcaggatattagggaacaaatagttactaaaaatgactttatgacagttttaaacgcccagtataacaaagtaaacaatatcattgagttctctccttcccaaatcaacctactattttctatcgtcgcaaattcaaaggaaaacagaagattaagaaagagaaatcaagatcgagatgacgagctattaaatgatcaccattatgattcagatattgatttttttatccataatgagtatttgcatggagtaagcagatccagaaaaaatttagaaagttttaatgactattatcatgatctctcggatggatttgaccaagactctggtgttaaaaaagcttcaaaagcgagtactggcttgtttgaatctgtatttggaggtaaaaaagataaagcaacgatgcgttctgacttaacaattgaagatttcatgaaaattttgaacccaaattacctgaacgacttagttcaccaaatggaattgcaaaaaaatcaaaatgagtcattgtatattaattactacttttatccaattttcgattcgttgtacaatttctccttgggttctattgcgggttgtattggtgcaactgtagtatacccaatagactttataaaaacaaggatgcaagcccaaagatctttagcccaatacaaaaactcaattgattgtttgttgaagattatatcccgcgaaggaataaaaggtctctactctggcttagggccacaattaataggagttgctcctgaaaaggcgataaaattgactgtcaatgattttatgagaaacaggttgactgataaaaacggcaagctaagcctttttcctgaaattatttctggcgcttcagctggtgcatgtcaagttatatttactaatccgttagagattgtaaaaattaggctacaggtccaatccgactatgttggtgaaaacatacaacaagccaatgaaactgccactcaaatagtcaaaaaattaggactgaggggcttgtacaatggtgtagccgcatgtttaatgagagatgttccattctctgctatttattttcccacttatgcacatttaaaaaaagatctctttgattttgatccaaatgataaaacaaagaggaatcgattaaaaacatgggagcttttaactgccggtgccattgctggtatgccagctgccttcttgactactccttttgatgttataaaaacaaggctccagatagatcctcgaaaaggtgagacaaagtataacggtatatttcatgctatccgaactatcttaaaggaagagagctttagaagctttttcaaaggtggtggagcccgtgtcctaagaagttctccccaatttgggttcactctggccgcctatgaattattcaagggctttattccctcccccgataacaaattaaaaagcagagagggtaggaagagattttgtatcgatgacgacgcaggcaatgaagagacagtagttcatagtaacggtgaactcccacagcaaaagttttactctgatgatagaaaacatgccaattattactataaaagctgtcaaattgcgaaaacattcattgatttggacaataacttttctaggtttgactcttcagtttataaaaactttcaagagcacctaagaagcattaacgggtga

SEQ ID NO: 23 - DNA, 879 bp, Saccharomyces cerevisiae
atggaggacagtaaaaagaaaggattaatagaaggcgctatactcgatataataaacggttccattgcaggcgcctgtggtaaggtgatcgagtttcctttcgatactgtgaaagtcaggttgcaaacacaagcatccaacgtgttcccaacaacatggtcttgtataaaatttacttaccaaaatgaaggaatagcacgagggttttttcaaggcattgcttcacctttagttggagcatgtctggagaacgcgacattatttgtgtcttataaccaatgttctaaatttttagaaaaacatacaaacgtttccccgttggggcaaatcctgatctctggtggagtagcgggttcatgtgctagtttagttttgacacccgtggagctggtgaagtgtaagttgcaggttgcgaacttacaagttgcatcagctaaaacgaaacatacaaaggtgttgcctacaataaaagcaattataactgagagaggattggcaggattgtggcaagggcaatcgggcacttttattcgagaaagcttcggtggtgttgcctggtttgcaacctacgaaatagttaagaagtcgttgaaagataggcactcccttgatgacccaaaaagagatgaaagtaagatatgggaactacttattagtggagggagcgctggattggcattcaacgccagtatttttcctgcggatactgtgaaatcagtaatgcaaactgaacatataagcctcaccaatgcggtgaagaagatatttggcaaatttggactaaagggtttttatcgaggactgggtataaccctttttagggcagtaccagcaaacgctgcagttttttacatctttgagactctttctgcactttaa

SEQ ID NO: 24 - DNA, 1332 bp, Escherichia coli
atggtaaaggaacgtaaaaccgagttggtcgagggattccgccattcggttccctatatcaatacccaccggggaaaaacgtttgtcatcatgctcggcggtgaagccattgagcatgagaatttctccagtatcgttaatgatatcgggttgttgcacagcctcggcatccgtctggtggtggtctatggcgcacgtccgcagatcgacgcaaatctggctgcgcatcaccacgaaccgctgtatcacaagaatatacgtgtgaccgacgccaaaacactggaactggtgaagcaggctgcgggaacattgcaactggatattactgctcgcctgtcgatgagtctcaataacacgccgctgcagggcgcgcatatcaacgtcgtcagtggcaattttattattgcccagccgctgggcgtcgatgacggcgtggattactgccatagcgggcgtatccggcggattgatgaagacgcgatccatcgtcaactggacagcggtgcaatagtgctaatggggccggtcgctgtttcagtcactggcgagagctttaacctgacctcggaagagattgccactcaactggccatcaaactgaaagctgaaaagatgattggtttttgctcttcccagggcgtcactaatgacgacggtgatattgtctccgaacttttccctaacgaagcgcaagcgcgggtagaagcccaggaagagaaaggcgattacaactccggtacggtgcgctttttgcgtggcgcagtgaaagcctgccgcagcggcgtgcgtcgctgtcatttaatcagttatcaggaagatggcgcgctgttgcaagagttgttctcacgcgacggtatcggtacgcagattgtgatggaaagcgccgagcagattcgtcgcgcaacaatcaacgatattggcggtattctggagttgattcgcccactggagcagcaaggtattctggtacgccgttctcgcgagcagctggagatggaaatcgacaaattcaccattattcagcgcgataacacgactattgcctgcgccgcgctctatccgttcccggaagagaagattggggaaatggcctgtgtggcagttcacccggattaccgcagttcatcaaggggtgaagttctgctggaacgcattgccgctcaggcgaagcagagcggcttaagcaaattgtttgtgctgaccacgcgcagtattcactggttccaggaacgtggatttaccccagtggatattgatttactgcccgagagcaaaaagcagttgtacaactaccagcgtaaatccaaagtgttgatggcggatttagggtaa

SEQ ID NO: 25 - DNA, 777 bp, Escherichia coli
atgatgaatccattaattatcaaactgggcggcgtactgctggatagtgaagaggcgctggaacgtctgtttagcgcactggtgaattatcgtgagtcacatcagcgtccgctggtgattgtgcacggcggcggttgcgtggtggatgagctgatgaaagggctgaatctgccggtgaaaaagaaaaacggcctgcgggtgacgcctgctgatcagatagacattatcaccggagcactggcgggaacggcaaataaaaccctgttggcatgggcgaagaaacatcagattgcggccgtaggtttgtttctcggtgacggcgacagcgtcaaagtgacccagcttgatgaagagttaggtcatgttggactggcgcagccaggttcgcctaagcttatcaactccttgctggagaacggttatctgccggtggtcagctccattggcgtaacagacgaagggcaactgatgaacgtcaatgccgaccaggcggcaacggcgctggcggcaacgctgggcgcggatctgattttgctctccgacgtcagcggcattctcgacggcaaagggcaacgcattgccgaaatgaccgccgcgaaagcagaacaactgattgagcagggcattattactgacggcatgatagtgaaagtgaacgcggcgctggatgcggcccgcacgctgggccgtccggtagatatcgcctcctggcgtcatgcggagcagcttccggcactgtttaacggtatgccgatgggtacgcggattttagcttaa

SEQ ID NO: 26 - DNA, 1074 bp, Corynebacterium glutamicum
atgataatgcacaatgtctatggtgttacaatgactattaaggtcgcaatcgcaggtgcctcaggttacgcaggtggtgaaatcttgagattgttattgggtcatccagcatatgcctctggtgaattagaaataggtgcattgaccgctgcatccactgccggtagtacattgggtgaattgatgccacatattcctcaattagctgatagagttatacaagacactacagctgaaacattggcaggtcatgatgttgtctttttaggtttgccacacggtttctcagcagaaatagccttacaattgggtcctgatgtcacagtaatcgattgtgccgctgactttagattacaaaatgcagccgactgggaaaaattctatggttccgaacatcaaggtacctggccatacggtattccagaaatgcctggtcacagagaagccttgagaggtgctaagagagttgcagtcccaggttgctttcctacaggtgctaccttagcattattgccagccgttcaagctggtttgatcgaacctgatgtatctgtagtttcaattaccggtgtttccggtgcaggtaaaaaggctagtgttgccttattgggttctgaaactatgggttcattgaaggcatacaacacctcaggtaaacatagacacactccagaaatcgctcaaaacttgggtgaagtttctgacaaaccagtaaaggtttcattcacacctgttttagctccattgcctagaggtattttaaccactgctacagcacctttgaaagaaggtgtcaccgccgaacaagccagagctgtttacgaagaattctacgctcaagaaactttcgtccatgtattaccagaaggtgcccaacctcaaacacaagctgttttgggttccaacatgtgtcacgttcaagtcgaaattgatgaagaagctggtaaagtattggttactagtgcaatcgacaatttgactaagggtacagcaggtgctgcagttcaatgcatgaacttatctgtcggttttgatgaagccgctggtttgccacaagtcggtgtagctccttaa

SEQ ID NO: 27 - DNA, 1176 bp, Corynebacterium glutamicum
atgtctacattggaaacctggcctcaagtcatcatcaacacatacggtactcctcctgtcgaattggtctctggtaaaggtgctacagtaaccgatgaccagggtaacgtttacatcgatttgttggctggtatagcagttaacgccttgggtcatgctcacccagcaataatcgaagctgtaactaaccaaataggtcaattgggtcatgtttctaacttatttgcatcaagacctgttgtcgaagttgccgaagaattaattaagagattctctttggatgacgcaacattagctgcacaaaccagagttttcttttgtaattcaggtgcagaagccaacgaagccgcttttaaaatcgctagattgacaggtagatccagaattttagcagccgttcatggtttccacggtagaaccatgggtagtttggcattaactggtcaaccagataagagagaagcatttttgccaatgccttccggtgttgaattctatccttacggtgacactgactatttgagaaaaatggtcgaaaccaatccaactgatgtagctgcaatctttttagaacctattcaaggtgaaacaggtgtagttccagcccctgaaggtttcttgaaggctgttagagaattgtgtgatgaatacggtatcttgatgatcactgacgaagtacaaacaggtgttggtagaaccggtgactttttcgcacatcaacacgatggtgtcgtaccagacgttgtcactatggctaaaggtttgggtggtggtttacctattggtgcctgcttggctacaggtagagccgctgaattaatgaccccaggtaaacatggtactacatttggtggtaaccctgttgcttgtgcagccgctaaagcagtcttgtcagtagttgatgacgcattttgcgccgaagttgctagaaagggtgaattattcaaggaattgttggctaaggttgatggtgtcgtagacgtcagaggtagaggtttgatgttaggtgttgtcttggaaagagatgtcgcaaagcaagccgtattggacggttttaaacacggtgttattttaaatgctccagcagataacatcattagattgactccacctttagtcataacagatgaagaaattgccgacgctgttaaagcaattgccgaaacaatagcttaa

SEQ ID NO: 28 - DNA, 1167 bp, Corynebacterium glutamicum
atggccgaaaaaggtataacagctccaaaaggtttcgttgcctctgctactacagccggtatcaaggcttcaggtaatccagatatggcattggttgtcaaccaaggtcctgaattttctgctgcagccgttttcactagaaatagagtctttgctgcacctgttaaagtctctagagaaaacgttgctgatggtcaaattagagctgtcttgtataatgctggtaatgcaaacgcctgtaacggtttacaaggtgaaaaggatgcaagagaatccgtaagtcatttggcccaaaatttgggtttagaagattccgacatcggtgtttgcagtacaggtttgattggtgaattgttgccaatggataagttgaacgctggtatcgaccaattgaccgccgaaggtgctttaggtgacaacggtgccgctgcagccaaagctatcatgaccactgataccgttgacaaggaaactgtagtttttgcagatggttggacagtaggtggtatgggtaaaggtgttggtatgatggcaccttcattggccaccatgttagtatgtttaacaaccgatgcctccgttactcaagaaatggctcaaattgctttggcaaatgccaccgctgtcactttcgacacattagatatagacggttctacatcaaccaacgatactgttttcttgttagcatctggtgcctcaggtatcactccaacacaagatgaattgaatgacgctgtttacgctgcatgctctgatattgccgctaaattacaagcagacgccgaaggtgttacaaagagagtagcagttaccgtcgtaggtactacaaataacgaacaagctattaatgcagccagaacagttgcaagagataacttgtttaaatgtgccatgttcggttctgacccaaattggggtagagtcttagctgcagttggtatggctgatgcagacatggaacctgaaaagatatccgtctttttcaacggtcaagctgtatgcttggatagtactggtgctcctggtgcaagagaagtcgacttgtctggtgctgatattgacgttagaatagatttgggtacttcaggtgaaggtcaagcaacagttagaaccactgatttgtcctttagttacgtcgaaattaattccgcttactcttcataa

SEQ ID NO: 29 - DNA, 1017 bp, Saccharomyces cerevisiae
atgtcaaccacagcatccacgccttcatctttacgtcatttgatttctataaaagatctttctgatgaagaattcagaatcttagtacaaagagctcaacatttcaagaatgtttttaaagcaaataaaacgaatgatttccaatccaaccatctgaaactattgggtagaactatagccttaatatttactaaaagatcaactagaacgagaatttcgaccgaaggtgcagccaccttctttggtgcccaaccgatgtttttaggtaaagaggatattcagcttggtgtcaatgaatcattttacgataccaccaaggttgtatcatctatggtttcatgtatttttgcccgtgtgaacaaacatgaagacatacttgctttttgcaaggattcctctgtaccgatcatcaactctctatgtgacaaattccaccctttgcaagcaatttgtgatcttttaacaataatcgaaaacttcaatatatctctagatgaagtaaataagggaatcaattcaaaattgaagatggcatggattggtgatgccaataatgtcataaatgatatgtgcatcgcatgtctgaaattcggtataagtgtcagtatttccactccccccggtattgaaatggattccgatattgtcgatgaagcaaagaaagttgctgagagaaacggtgcgacatttgaattaacacacgactctttaaaggcctccaccaatgccaatatattagtaaccgatactttcgtttccatgggtgaagaatttgcgaaacaggccaagctgaaacaattcaaaggttttcaaatcaatcaagaacttgtctctgtggctgatccaaactacaaatttatgcattgtctgccaagacatcaagaagaagttagtgatgatgtcttttatggagagcattccatagtctttgaagaagcagaaaacagattatatgcagctatgtctgccattgatatctttgttaataataaaggtaatttcaaggacttgaaataa

SEQ ID NO: 30 - DNA, 1275 bp, Saccharomyces cerevisiae
atgtccgaagctaccctctcctccaagcaaaccattgaatgggaaaacaaatactccgcccacaactaccaccccttgcccgtcgtttttcacaaggctaagggcgcacatgtgtgggacccggagggtaagctgtacctcgacttcctgagcgcttattctgccgtcaaccagggccattgccatcctcacatcatcaaggctttgacggagcaagcacaaacactaacattgtcctccagagcgttccacaacgatgtttacgcgcaattcgccaagttcgtgaccgaattcttcgggttcgaaaccgttttgcccatgaacaccggtgcagaagccgtggaaactgctttgaagttggccagaagatgggggtacatgaagaagaacatccctcaagataaagccatcattctgggtgccgagggtaacttccacgggagaaccttcggtgctatcagtttgagtaccgactacgaggactccaagttgcatttcgggcctttcgtgcctaacgttgccagtggtcactccgtgcacaagatcagatacggccacgcagaagatttcgtccctatcttggaatctcctgaaggtaagaacgttgccgccatcattctagagccaattcagggtgaagccggtatcgtcgtgccccccgcagactacttcccaaaggtctccgcattatgccgtaagcacaacgtcctattgatcgttgacgaaattcaaaccggtatcggtagaaccggtgagttgctttgctacgaccactacaaggcagaggccaagcctgatattgttttgttaggtaaggctctctcaggtggtgttcttcccgtctcatgtgttctgtcttctcacgacatcatgtcttgctttaccccaggatctcacggttctactttcggcggtaatcctttggcttcccgcgttgccatcgccgccctcgaggtcatccgcgacgagaagctgtgccaaagagccgcccaactgggtagctctttcatcgcccaattgaaagctctccaagccaaatctaacggtataatctctgaggtgcgtggtatgggactgcttaccgccatcgtaatcgacccatccaaggccaatggtaagaccgcttgggacttgtgtctattgatgaaggatcacggcctcttggctaagcccacccacgaccacatcatcagattggctcctcctttggtcatctccgaagaggacttgcaaaccggtgtcgaaaccattgccaagtgtatcgatctgttataa

SEQ ID NO: 31 - DNA, 1401 bp, Saccharomyces cerevisiae
atgtctagtactcaagtaggaaatgctctatctagttccactactactttagtggacttgtctaattctacggttacccaaaagaagcaatattataaagatggcgagacgctgcacaatcttttgcttgaactaaagaataaccaagatttggaacttttaccgcatgaacaagcgcatcctaaaatatttcaagcgctcaaggctcgtattggtagaattaataatgaaacgtgcgaccccggtgaggagaactcgtttttcatatgcgatttgggagaagtcaagagattattcaacaactgggtgaaggagcttcctagaattaagccattttatgccgtcaaatgtaatcctgataccaaggttttgtcattattagcagagttgggcgttaatttcgattgcgcttccaaagtggaaattgacagagtattatcgatgaacatctcgccggatagaattgtttacgctaatccttgtaaagtagcatctttcattagatatgcagcttcaaaaaatgtaatgaagtctacttttgacaatgtagaagaattgcataaaatcaaaaagtttcatcctgagtctcagttgttattaagaatcgctaccgatgactctaccgctcaatgtcgactttccaccaaatatggctgtgaaatggaaaacgtagacgttttattaaaggctataaaggaactaggtttaaacctggctggtgtttctttccacgtcggttcaggcgcttctgattttacaagcttatacaaagccgttagagatgcaagaacggtatttgacaaagctgctaacgaatacgggttgccccctttgaagattttggatgtaggtggtggatttcaatttgaatccttcaaagaatcaactgctgttttgcgtctagcgctagaggaatttttccctgtaggttgtggtgttgatataattgcagagcctggcagatactttgtagctacagcgttcactttggcatctcatgtgattgcgaagagaaaactgtctgagaatgaagcaatgatttacactaacgatggtgtatacgggaacatgaattgtattttattcgatcatcaagagccccatccaagaaccctttatcataatttggaatttcattacgacgattttgaatccactactgcggtcctcgactctatcaacaaaacaagatctgagtatccatataaagtttccatctggggacccacatgtgatggtttggattgtattgccaaagagtattacatgaagcatgatgttatagtcggtgattggttttattttcctgccctgggtgcctacacatcatcggcggctactcaattcaacggctttgagcagactgcggatatagtatacatagactctgaactcgattga

SEQ ID NO: 32 - DNA, 879 bp, Saccharomyces cerevisiae
atgtatgaagtaatacagaaaaggaaaacaaaaataataaacgttttacagagtcctgaactcatgaggctcatagaggacccatcaaatctgggtatttctttacattttccagtaagttcactgctaaaaagtaataagtgcacaccaatgcctaaactttctacgtatagtttggctagtgggggatttaaggattggtgcgcggacatccctctagacgttccaccagagattgatatcatcgatttttactgggatgttattttatgcatggaatctcaattcatattagattacaatgttccgtcaaaaaataaggggaacaatcagaagtctgttgctaagctgttgaaaaataagcttgtaaacgatatgaaaactacgttaaaaagactaatttataatgaaaataccaagcaatataaaaataataatagccacgatggttacaattggagaaaactaggctcgcagtatttcatactgtatcttcccctatttacgcaggaactgatttggtgtaaacttaatgaaaactatttccatgttgtattaccatctttactgaatagtaggaacgttcatgataaccacagtacctatataaataaagattggttacttgcccttttagagctaacttccaacctgaaccaaaacttcaaattcgaatacatgaaattgagattgtatattttaagagatgatttaattaataatggtttggatcttttgaaaaatcttaactgggtcggtgggaaactgattaaaaatgaagatagagaagtcttgttgaactcgaccgatttagctacggattctatttctcatttattaggtgatgaaaactttgttattttagagtttgaatgctaa

SEQ ID NO: 33 - DNA, 1191 bp, Saccharomyces cerevisiae
atgactgtcaccataaaagaattgactaaccacaactacattgaccacgaactatcagccactttagactcaacggatgcgttcgagggtcccgagaagttgctggaaatctggttcttccctcacaagaagtccatcacgaccgaaaagacattaagaaatattggcatggatagatggatcgagattttgaaattagtgaaatgcgaagttctttccatgaagaagactaaagaactggatgcctttttgttgagtgagtcttccctcttcgtcttcgatcacaaattgacgatgaagacgtgcggtactacaaccacattgttctgtctcgaaaagcttttccagatcgttgagcaagagttatcgtgggctttccgcacaacacaagggggcaagtacaaaccatttaaagtgttttattctagacgatgtttccttttcccctgtaagcaagccgctatccatcaaaactgggctgacgaagtcgactatttgaacaaatttttcgacaatggtaaaagttattccgtgggaagaaatgacaagagcaaccactggaacctgtacgtcaccgagacggaccgctccacacctaagggaaaggagtacatcgaggatgacgacgaaactttcgaagtactgatgacggagctggacccagaatgcgctagtaagtttgtttgcgggcctgaggcatccacaaccgctctcgtggagccaaacgaagataagggccacaacctcggctaccaaatgactaaaaatacaaggcttgacgaaatatatgtcaactcggcccaagactccgatttatcatttcaccacgatgcatttgcgttcacgccatgtggatactcatccaatatgattctcgctgaaaaatactattacaccctgcacgtgactccggaaaagggttggtcttacgcctctttcgaaagtaacatacccgtatttgacatttcccaagggaagcaagacaacttggacgttcttctacatattctgaacgtttttcaaccaagagagttctcgatgaccttttttaccaaaaattatcagaaccaatccttccaaaaactactaagcatcaacgagtcactgcccgactacatcaagttagacaaaattgtttatgatctggacgactaccaccttttctatatgaaattgcagaagaaaatatga

SEQ ID NO: 34 - DNA, 882 bp, Saccharomyces cerevisiae
atggcacaagaaatcactcacccaactattgtagacggctggttcagagaaatttctgataccatgtggccaggccaggccatgactttaaaagtggagaaagttttacaccatgagaagtcaaaatatcaagacgttttgatcttcaaatccactacatatggtaatgttctagttttagataatgtaattcaagccaccgaaagggatgaatttgcctaccaagaaatgattgcccatcttgccttgaattcccatccaaatcctaagaaggttcttgttattggtgggggtgatggtggtgttttgagagaggttgtcaagcatgattccgttgaggaagcctggttatgtgacattgatgaagctgttattagactatcaaaggagtacctaccagaaatggctgcctcttattctcacccaaaggttaagacccacattggtgatggtttccaatttttaagagattaccaaaacacatttgacgtaatcattactgactcttctgacccagaaggtccagctgaaacccttttccaaaaggaatatttccaattgttgaacagtgcgttgacagaaaagggtgtaatcactacacaagcagaaagtatgtggattcacttgccaatcattaaggacttaaagaaagcctgttctgaagttttcccagttgcagaatactctttcgttactattccaacttacccaactggtacgattggttttatggtttgctccaaagataaaacttgcaatgtcaagaagccactacgtgaaatctctgatgagaaggaggctgaattatacagatactataacaagaaaattcacgaagcttcctttgttctaccaacctgggcagccaaggaattaaattag

SEQ ID NO: 35 - DNA, 1527 bp, Saccharomyces cerevisiae
atgaatacagtttcaccagccaaaaaaaaggttattataattggtgccggtattgctgggcttaaagctgcatctacgctacaccaaaacggtattcaagattgtcttgttcttgaggccagagatcgggtcggtggtaggttgcaaactgtcacaggctatcaaggtcggaaatatgatataggtgctagctggcaccatgatacgttgacaaaccctttatttttggaagaggctcaactgagtttgaatgatgggagaacgaggtttgtttttgatgacgataattttatttatatcgacgaagaacgtggaagggtagaccatgacaaggaactgcttcttgaaattgtggacaatgaaatgagcaaattcgcagagttagaattccatcaacacttaggagtttcagattgctccttttttcaattagtaatgaaatacttactacaaagacgccaatttctcacaaatgaccaaataagatatttgccacaactctgtcgatatctggaattgtggcacggcttagattggaagcttttgagtgccaaggatacatacttcggtcaccaaggaaggaacgcctttgctttgaactatgattctgtggttcaaagaattgctcaaagctttcctcaaaattggttaaagctaagttgtgaagtgaaatcaattacacgagaaccttcaaaaaatgtgacagtgaactgtgaagatggtactgtgtacaatgctgattatgttattattacagtacctcaaagtgtattgaatttgtctgtacaacctgaaaaaaatttacggggaagaatagaatttcaaccacccttgaaaccagtgattcaagatgcttttgacaagatccattttggagcgctaggtaaagtaatttttgagtttgaagaatgttgttggtcgaacgaaagttcaaaaattgtaactttggctaactctaccaatgaatttgtcgaaatagtacgtaatgcggaaaatttagatgaattagactctatgctagaaagggaagattctcaaaagcatacgagtgttacttgttggagccagcctttatttttcgtaaatttgtcaaaaagcacaggagtagcaagctttatgatgttgatgcaggcaccgcttacaaatcacatagaatccattagagaagataaagagcgtctttttagttttttccaacctgtgctgaacaagattatgaagtgtctagattctgaggatgtcatcgacggaatgaggccgatagaaaacattgcaaacgctaataaaccagtcttaagaaacatcatcgttagcaactggacacgcgatccttactcacgcggtgcttattcggcctgttttccaggagatgatccagttgatatggttgttgcaatgtctaatggtcaagactcccgcataagatttgcaggcgaacatactatcatggacggcgccggctgtgcctatggtgcttgggaaagcggaagacgggaggcgactcgaatctctgacttactgaaatag

SEQ ID NO: 36 - DNA, 1014 bp, Saccharomyces cerevisiae
atgaacaggattaagaatacattttctgttgctaagagattaaaactaagcaaagttatgacgaactcagaattaccgagcatattcgaaggaactgttgatttagggattattggtggtacaggtttatataaccttgactgtctggagcccatcgctttgcttccacccatggtaacaccatggggtaccacatcgtctcctgtcacaatctctcagttcgtaggaactaacagccactttcacgttgcgttcatagccagacacggtattaaccacgaatacccacccactaaagtcccatttagagcaaacatggcggccttaaagaacttaaattgtaaagccgttctttcttttagtgccgtggggtctttacaaccccatataaagcctagagattttgtgttaccacagcaaataatcgacagaactaaaggcataagacattcttcatatttcaacgatgaaggcttggtaggtcacgttggtttcggacagccgttctctcaaaaattcgcagagtatatctatcaattcaagaacgagataacaaatcctgaatccgaagaaccgtgccatttgcattacgacaaggatatgaccgttgtgtgtatggaaggcccacaattctccacgcgcgctgaatccaagatgtacagaatgtttggtggccatgttattaacatgagtgttattccagaagccaaattggcgcgtgagtgtgagctgccttaccagatgatttgtatgtctaccgattacgacgcatggagagatgaggcagaacctgttaccgtagaaaccgttattggtaatttgacgaataatgggcgcaatgcaaatattttagcttctaagatcatcgtctcaatggccaaggaaatcccagagttcatgcatactggcgatgggctgcgcggttccatcaagaaatctatctctaccaaaccagaggctatgtccaaggaaaccttagaaagactaagatacttatttccaaactattggtaa

SEQ ID NO: 37 - DNA, 1131 bp, Saccharomyces cerevisiae
atgaccttggcacccctagacgcctccaaagttaagataactaccacacaacatgcatctaagccaaaaccgaacagtgagttagtgtttggcaagagcttcacggaccacatgttaactgcggaatggacagctgaaaaagggtggggtaccccagagattaaaccttatcaaaatctgtctttagacccttccgcggtggttttccattatgcttttgagctattcgaagggatgaaggcttacagaacggtggacaacaaaattacaatgtttcgtccagatatgaatatgaagcgcatgaataagtctgctcagagaatctgtttgccaacgttcgacccagaagagttgattaccctaattgggaaactgatccagcaagataagtgcttagttcctgaaggaaaaggttactctttatatatcaggcctacattaatcggcactacggccggtttaggggtttccacgcctgatagagccttgctatatgtcatttgctgccctgtgggtccttattacaaaactggatttaaggcggtcagactggaagccactgattatgccacaagagcttggccaggaggctgtggtgacaagaaactaggtgcaaactacgccccctgcgtcctgccacaattgcaagctgcttcaaggggttaccaacaaaatttatggctatttggtccaaataacaacattactgaagtcggcaccatgaatgcttttttcgtgtttaaagatagtaaaacgggcaagaaggaactagttactgctccactagacggtaccattttggaaggtgttactagggattccattttaaatcttgctaaagaaagactcgaaccaagtgaatggaccattagtgaacgctacttcactataggcgaagttactgagagatccaagaacggtgaactacttgaagcctttggttctggtactgctgcgattgtttctcccattaaggaaatcggctggaaaggcgaacaaattaatattccgttgttgcccggcgaacaaaccggtccattggccaaagaagttgcacaatggattaatggaatccaatatggcgagactgagcatggcaattggtcaagggttgttactgatttgaactga

SEQ ID NO: 38 - DNA, 564 bp, Saccharomyces cerevisiae
atgtctatagcaagttatgcccaagagttgaagttggctttacatcaatatccaaacttccctagtgaaggcattctcttcgaagatttcttacccattttcaggaacccaggtcttttccagaagttgatcgatgctttcaaactgcatttagaagaagcttttccagaagttaaaattgattatatcgtcgggttggaatcccgtgggttcttgttcggaccaactttagctttggccctaggtgttggtttcgttccagtcaggaaggcaggtaagctacctggcgaatgttttaaggctacgtacgaaaaggagtacggttctgatctttttgagatacagaaaaacgctattccagcaggttccaacgttatcattgttgatgacattattgccactggtggttctgctgctgcagccggcgaattagttgaacaactcgaagccaaccttttggaatataactttgttatggagttggatttcttgaaaggcaggagtaagttgaatgctccagtgttcactttactgaacgctcaaaaggaagcgttgaaaaaatga

SEQ ID NO: 39 - DNA, 1491 bp, Saccharomyces cerevisiae
atgtcaatgagtaatattgttgtttttggaggggactcgcaccccgagttagttactaagatctgtgaaaatttggacattcacccatcgaaagtagaattagggaagttttctaatggggaaacgaacattgctcttcgcgaatctgttcgtgaaaaggatgtatatatcatccagagtggttgtggccaggtgaacgacacgttcatgcagttgctgattttaatcagtgcctgcaagtccgcttctgcctcgagggttacagccgtaatgccatatctctgctactcgagacagccagatattccatatactgccaagggtgctcccataatttccaagcctaaagaaaactatacttttgaatcgcatccaggcacacccgtgtcatcttctttaatgacgcaaagaccaggtgctgagagctcgttgaagagtttggatagtgcaatacgatcaactatcaacttagaaaatcctcaacctatcagaacaccaaacagcagtgctacggcgaataacaatttcgacatcaagaagacgctttctttttcaagaattcctatgattcccggtggtaagttacaaaatacaagcaatagcacggacgctggtgaattgttcaacgctcaaaatgcaggctataagctatgggtagtacaagccggtactttgattgctcatttgttgagtgctgcaggtgctgaccatgtgatcacaatggatttgcacgatccacagttccctgggttttttgacattccagtggataatctctactgtaaacccattgcacaaaactacatccagcatcgcattccagattatcaggatgctgtgatcgtttctccagatgctggtggtgcaaagagagctacggctattgcagacgccttggaattgtccttcgccctaattcataaagaaagaagatctcagttattgaagggccctccagatgcgacgttaacctctggtggttcgttaccagtatctccaaggccattagttactactttggtttcctcccaaaatactacttcttcaggtgccactggggttgcggcccttgaaatgaagaaaacaacttcaacatcttccacctcgtcgcaatcttctaattcgtccaagttcgttcaaactaccatgcttgttggcgatgttagaaacaaggtgtgtattatagtcgacgacttggtggatacttcatacactattacaagagctgcgaaattgttgaaggatcaaggatctaccaaagtttatgccttaataacgcacggtgttttttccggtgatgcgctagaaagaatcggccaaagtagtatagataagttgatcatttctaacacggttcctcaagatagaacactacagtacctaggtaaggacagagtggatgttattgatgtctcctgcataatcggtgaagcaattagaagaatccataacggtgaatccatttctatgttgttcgagcatggatggtag

SEQ ID NO: 40 - DNA, 1197 bp, Leishmania infantum
gttgtttctagaaaaacaatgtctgttcactctatcttgttctcttctgaacacgttactgaaggtcacccagacaagttgtgtgaccaagtttctgacgctgttttggacgcttgtttggctggtgacccattctctaaggttgcttgtgaatcttgtgctaagactggtatggttatggttttcggtgaaatcactactaaggctgttttggactaccaaaagatcgttagaaacactatcaaggacatcggtttcgactctgctgacaagggtttggactacgaatcttgtaacgttttggttgctatcgaacaacaatctccagacatctgtcaaggtttgggtaacttcgactctgaagacttgggtgctggtgaccaaggtatgatgttcggttacgctactgacgaaactgaaactttgatgccattgacttacgaattggctagaggtttggctaagaagtactctgaattgagaagagacggttctttggaatgggctagaccagacgctaagactcaagttactgttgaatacgactacgacactagagaaggtaagcaagttttgactccaaagagagttgctgttgttttgatctctgctcaacacgacgaacacgttactaacgacaagatctctgttgacttgatggaaaaggttatcaaggctgttatcccagctaacatgttggacgctgaaactaagtactggttgaacccatctggtagattcgttagaggtggtccacacggtgacgctggtttgactggtagaaagatcatcgttgacacttacggtggttggggtgctcacggtggtggtgctttctctggtaaggacccatctaaggttgacagatctgctgcttacgctgctagatggatcgctaagtctatcgttgctggtggtttggctagaagatgtttggttcaattggcttacgctatcggtgttgctgaaccattgtctatgcacgttgaaacttacggtactggtaagtacgacgacgctaagttgttggaaatcgttaagcaaaacttcaagttgagaccatacgacatcatccaagaattgaacttgagaagaccaatctactacgaaacttctcgtttcggtcacttcggtagaaaggacgaattgggtactggtggtttcacttgggaagttccaaagaagatggttgaataa

SEQ ID NO: 41 - DNA, 1044 bp, Saccharomyces cerevisiae
atggtttctgtggagtttttacaggagttaccaaaatgtgagcatcacttgcatttggaaggtactctagaacctgacctattgttcccattagctaaaagaaacgatataattctacctgaaggttttcctaaatcggtcgaggaattaaacgaaaagtataagaagtttcgtgatctgcaggatttcttagattactattatattggtactaatgtcttgattagtgaacaagatttctttgatttggcgtgggcctattttaaaaaagttcacaaacaaggcttggtccatgctgaagtgttttacgaccctcagtcacatacatctaggggcatctccatagaaacagtcactaaaggtttccaaagagcttgtgacaaagccttctctgaatttggtattacatccaagctaattatgtgtctgttaagacacattgaaccagaggaatgtttgaaaactatcgaagaagctaccccatttattaaagatggtactatctctgccttaggattagattctgctgagaaaccatttcccccacatttatttgttgaatgttacggaaaggccgcctcattgaataaagatttaaaactaactgcacacgcaggtgaagaaggccccgctcaattcgtctcggatgctttagacttgttgcaagtaacaagaatcgatcacggtatcaacagtcaatacgacgaggagttattggataggttgtcgcgcgaccagaccatgctaactatttgtcctctctccaacgtgaagctacaagtagtccaatccgtttcagagttaccactacaaaagtttcttgacagagatgttccattttctttaaattctgatgaccccgcctattttggtggttatatcttagatgtctacactcaagtttcgaaagatttcccacactgggaccatgaaacatggggtcgtatcgctaagaacgccattaaaggttcatggtgtgacgataaaagaaagaacggtttgttaagtagagtggacgaagtagtcactaaatattcgcattag

SEQ ID NO: 42 - DNA, 1857 bp, Saccharomyces cerevisiae
atgccagagtatacgctactggctgataatataagggagaatatcgttcatttcgatccgaatggtttgtttgataacttgcacaccattgttcatgaagatgacagtcaagagaacgaggaggccgagcatttcaattatgatcaggtgttggataaatcgttattgtcaagaggttctattgtcggtctcggtttaggactaatgagtcccgttttaggaatgtgcactagtatggccattgggctaattaatggtggtccgttaactataatgctaggttttttaatcagtggagtgtgtatatggttttcgtcgctttctcttggtgagattgtttcaaaatttccgatggaactgcatgttgggagtgccatgttggccccggagaaattgaaattagtatgttcgtggtacactggctggttaatgctcatagggaattggactatgagtaccagtattacttttgcaggcgctcaacttaccatttctttgattctgatgacgaactccaacctaatatccgaggcacacttgattttttacacagtcattgtattttacttagttgtgactgttgtaggcctcgtgaatttgaaatttgcaagatttattgaaacaataaacaaagtctgtgtttattggatcatatatgccattatatttattgatattcttctactagtattccacaaaggtaaatttcgatctttgaagtacgcgctatttcactttgataataatctatcagggtataaaagcgcatttctttccttcatcattggattccaacagtctaatttcacgttacaaggtttcagtatgttacctgctttagctgacgaagtcaaagttcctgagaaggatattccacgtggtatgtcgaatgcggtattgttatccgcgttctctggagtcatttttcttataccaataatgttaatcctgccagataatgatttgctttttaccaatcataaggttctaccaatagtgaacatttttacaaaatcgactgattcggtggtcttgtctttttttttagtgctcctaattttaggaaacttactgttttccggaattggctcgattactacatcttctcgtgcggtatatagttttagtcgtgaccaggctataccatactacgataaatggacctacgtcgaaccggattctcagtcaaaagtccccaagaattctgttgtattgagtatgataatatcatactttttaggtctgctagctttgatttcaacggccgcatttaatgcttttataggcgctgcagtgctctgtctttgttctgcgactttcattccgttagtcttggtgctgtttacgagaagaagagctatccgaagcgcgccagtaaaaatcaggtataagtttggttggttcatcaacattgtttctattgtgtggctcttgttatctatggtttctgtttgcctaccaacgcaagtgcctgtaactttcaaaacaatgaattatgctttaatggtgtacgtattctgcattttagttatcactggtctttatttcaaatgggggaagtataattttagattacccttggcagatgacatcaaggctccaattcccagtgatgcggaagaaactgtttttgaactagaggatagcaatgttgaacatactctaaactcgggaaccacagtgaaagagtctgtagaaaataattctgaagaaggtttcatcaaggtgcatcctaaaagtagtacagaaaatccctttgaggaaaatgaggaaaacgtgataaccgattatggtgatgagcaccatacagcagaacaagaatttgatcttgccgatgatcgtagatatgatatatga

SEQ ID NO: 43 - DNA, 2088 bp, Saccharomyces cerevisiae
atgactgtcaccataaaagaattgactaaccacaactacattgaccacgaactatcagccactttagactcaacggatgcgttcgagggtcccgagaagttgctggaaatctggttcttccctcacaagaagtccatcacgaccgaaaagacattaagaaatattggcatggatagatggatcgagattttgaaattagtgaaatgcgaagttctttccatgaagaagactaaagaactggatgcctttttgttgagtgagtcttccctcttcgtcttcgatcacaaattgacgatgaagacgtgcggtactacaaccacattgttctgtctcgaaaagcttttccagatcgttgagcaagagttatcgtgggctttccgcacaacacaagggggcaagtacaaaccatttaaagtgttttattctagacgatgtttccttttcccctgtaagcaagccgctatccatcaaaactgggctgacgaagtcgactatttgaacaaatttttcgacaatggtaaaagttattccgtgggaagaaatgacaagagcaaccactggaacctgtacgtcaccgagacggaccgctccacacctaagggaaaggagtacatcgaggatgacgacgaaactttcgaagtactgatgacggagctggacccagaatgcgctagtaagtttgtttgcgggcctgaggcatccacaaccgctctcgtggagccaaacgaagataagggccacaacctcggctaccaaatgactaaaaatacaaggcttgacgaaatatatgtcaactcggcccaagactccgatttatcatttcaccacgatgcatttgcgttcacgccatgtggatactcatccaatatgattctcgctgaaaaatactattacaccctgcacgtgactccggaaaagggttggtcttacgcctctttcgaaagtaacatacccgtatttgacatttcccaagggaagcaagacaacttggacgttcttctacatattctgaacgtttttcaaccaagagagttctcgatgaccttttttaccaaaaattatcagaaccaatccttccaaaaactactaagcatcaacgagtcactgcccgactacatcaagttagacaaaattgtttatgatctggacgactaccaccttttctatatgaaattgcagaagaaaataggatctggttctggttctatggcacaagaaatcactcacccaactattgtagacggctggttcagagaaatttctgataccatgtggccaggccaggccatgactttaaaagtggagaaagttttacaccatgagaagtcaaaatatcaagacgttttgatcttcaaatccactacatatggtaatgttctagttttagataatgtaattcaagccaccgaaagggatgaatttgcctaccaagaaatgattgcccatcttgccttgaattcccatccaaatcctaagaaggttcttgttattggtgggggtgatggtggtgttttgagagaggttgtcaagcatgattccgttgaggaagcctggttatgtgacattgatgaagctgttattagactatcaaaggagtacctaccagaaatggctgcctcttattctcacccaaaggttaagacccacattggtgatggtttccaatttttaagagattaccaaaacacatttgacgtaatcattactgactcttctgacccagaaggtccagctgaaacccttttccaaaaggaatatttccaattgttgaacagtgcgttgacagaaaagggtgtaatcactacacaagcagaaagtatgtggattcacttgccaatcattaaggacttaaagaaagcctgttctgaagttttcccagttgcagaatactctttcgttactattccaacttacccaactggtacgattggttttatggtttgctccaaagataaaacttgcaatgtcaagaagccactacgtgaaatctctgatgagaaggaggctgaattatacagatactataacaagaaaattcacgaagcttcctttgttctaccaacctgggcagccaaggaattaaattag

SEQ ID NO: 44 - DNA, 27 bp, Artificial Sequence
  Primer
gtatgaagtaatacagaaaaggaaaac

SEQ ID NO: 45 - DNA, 46 bp, Artificial Sequence
  Primer
gacctgcagcgtacgaagcttcagcaatctctggtggaacgtctag

SEQ ID NO: 46 - DNA, 24 bp, Artificial Sequence
  Primer
ctgaagcttcgtacgctgcaggtc

SEQ ID NO: 47 - DNA, 24 bp, Artificial Sequence
  Primer
ggccactagtggatctgatatcac

SEQ ID NO: 48 - DNA, 52 bp, Artificial Sequence
  Primer
gtgatatcagatccactagtggcccctgaaccaaaacttcaaattcgaatac

SEQ ID NO: 49 - DNA, 25 bp, Artificial Sequence
  Primer
gcattcaaactctaaaataacaaag

SEQ ID NO: 50 - DNA, 48 bp, Artificial Sequence
  Primer
gtgatatcagatccactagtggccatagcttcaaaatgtttctactcc

SEQ ID NO: 51 - DNA, 28 bp, Artificial Sequence
  Primer
tttgtaattaaaacttagattagattgc

SEQ ID NO: 52 - DNA, 54 bp, Artificial Sequence
  Primer
gcaatctaatctaagttttaattacaaaatgtctagtactcaagtaggaaatgc

SEQ ID NO: 53 - DNA, 51 bp, Artificial Sequence
  Primer
gtgctagtgtctcccgtcttctgttcaatcgagttcagagtctatgtatac

SEQ ID NO: 54 - DNA, 24 bp, Artificial Sequence
  Primer
acagaagacgggagacactagcac

SEQ ID NO: 55 - DNA, 26 bp, Artificial Sequence
  Primer
attttcaacatcgtattttccgaagc

SEQ ID NO: 56 - DNA, 54 bp, Artificial Sequence
  Primer
gcttcggaaaatacgatgttgaaaatcctgaaccaaaacttcaaattcgaatac

SEQ ID NO: 57 - DNA, 50 bp, Artificial Sequence
  Primer
gtgatatcagatccactagtggcctgccgtaaaccactaaatcggaaccc

SEQ ID NO: 58 - DNA, 26 bp, Artificial Sequence
  Primer
tagggcccacaagcttacgcgtcgac

SEQ ID NO: 59 - DNA, 51 bp, Artificial Sequence
  Primer
gtcgacgcgtaagcttgtgggccctactaatttaattccttggctgcccag

SEQ ID NO: 60 - DNA, 52 bp, Artificial Sequence
  Primer
ctactttttacaacaaatataacaaaatggcacaagaaatcactcacccaac

SEQ ID NO: 61 - DNA, 26 bp, Artificial Sequence
  Primer
tttgttatatttgttgtaaaaagtag

SEQ ID NO: 62 - DNA, 26 bp, Artificial Sequence
  Primer
acgcacagatattataacatctgcac

SEQ ID NO: 63 - DNA, 50 bp, Artificial Sequence
  Primer
gtgcagatgttataatatctgtgcgtatagcttcaaaatgtttctactcc

SEQ ID NO: 64 - DNA, 51 bp, Artificial Sequence
  Primer
cttcggaaaatacgatgttgaaaatgttactccgcaacgcttttctgaacg

SEQ ID NO: 65 - DNA, 28 bp, Artificial Sequence
  Primer
taaagtaagagcgctacattggtctacc

SEQ ID NO: 66 - DNA, 56 bp, Artificial Sequence
  Primer
ggtagaccaatgtagcgctcttactttatcatattttcttctgcaatttcatatag

SEQ ID NO: 67 - DNA, 56 bp, Artificial Sequence
  Primer
gtttcgaataaacacacataaacaaacaaaatgactgtcaccataaaagaattgac

SEQ ID NO: 68 - DNA, 30 bp, Artificial Sequence
  Primer
tttgtttgtttatgtgtgtttattcgaaac

SEQ ID NO: 69 - DNA, 26 bp, Artificial Sequence
  Primer
tcgagtttatcattatcaatactgcc

SEQ ID NO: 70 - DNA, 54 bp, Artificial Sequence
  Primer
ggcagtattgataatgataaactcgacctgaaccaaaacttcaaattcgaatac

SEQ ID NO: 71 - DNA, 54 bp, Artificial Sequence
  Primer
ggcagtattgataatgataaactcgagtttaaagattacggatatttaacttac

SEQ ID NO: 72 - DNA, 34 bp, Artificial Sequence
  Primer
ttttagtttatgtatgtgttttttgtagttatag

SEQ ID NO: 73 - DNA, 61 bp, Artificial Sequence
  Primer
ctataactacaaaaaacacatacataaactaaaaatggttaataattcacagcatccttac

SEQ ID NO: 74 - DNA, 53 bp, Artificial Sequence
  Primer
gactaataattcttagttaaaagcacttcattcattaatgaccttgtctgccc

SEQ ID NO: 75 - DNA, 27 bp, Artificial Sequence
  Primer
agtgcttttaactaagaattattagtc

SEQ ID NO: 76 - DNA, 26 bp, Artificial Sequence
  Primer
aggtatcatctccatctcccatatgc

SEQ ID NO: 77 - DNA, 54 bp, Artificial Sequence
  Primer
gcatatgggagatggagatgatacctcctgaaccaaaacttcaaattcgaatac

SEQ ID NO: 78 - DNA, 45 bp, Artificial Sequence
  Primer
cgtcctctcgaaaggtggtttaaagattacggatatttaacttac

SEQ ID NO: 79 - DNA, 56 bp, Artificial Sequence
  Primer
ctacaaaaaacacatacataaactaaaaatgaacaggattaagaatacattttctg

SEQ ID NO: 80 - DNA, 53 bp, Artificial Sequence
  Primer
ggtcgacgcgtaagcttgtgggccctattaccaatagtttggaaataagtatc

SEQ ID NO: 81 - DNA, 38 bp, Artificial Sequence
  Primer
caggtggtcatggccctttgccgtaaaccactaaatcg

SEQ ID NO: 82 - DNA, 49 bp, Artificial Sequence
  Primer
ctacaaaaaacacatacataaactaaaaatgaccttggcacccctagac

SEQ ID NO: 83 - DNA, 49 bp, Artificial Sequence
  Primer
ggtcgacgcgtaagcttgtgggccctatcagttcaaatcagtaacaacc

SEQ ID NO: 84 - DNA, 60 bp, Artificial Sequence
  Primer
cattaaaaaactatatcaattaatttgaattaacttaccaatagtttggaaataagtatc

SEQ ID NO: 85 - DNA, 49 bp, Artificial Sequence
  Primer
catgactcgaggtcgacggtatctcagttcaaatcagtaacaacccttg

SEQ ID NO: 86 - DNA, 55 bp, Artificial Sequence
  Primer
gtaattatctactttttacaacaaatataacaaaatgaccttggcacccctagac

SEQ ID NO: 87 - DNA, 46 bp, Artificial Sequence
  Primer
caggtggtcatggccctttttgtaattaaaacttagattagattgc

SEQ ID NO: 88 - DNA, 58 bp, Artificial Sequence
  Primer
gcaatctaatctaagttttaattacaaaatgtccaagagcaaaactttcttatttacc

SEQ ID NO: 89 - DNA, 53 bp, Artificial Sequence
  Primer
ctaaatcattaaagtaacttaaggagttaaatttaaaattccaatttctttgg

SEQ ID NO: 90 - DNA, 32 bp, Artificial Sequence
  Primer
atttaactccttaagttactttaatgatttag

SEQ ID NO: 91 - DNA, 39 bp, Artificial Sequence
  Primer
caggtggtcatggcccttgcgaatttcttatgatttatg

SEQ ID NO: 92 - DNA, 21 bp, Artificial Sequence
  Primer
gcgaatttcttatgatttatg

SEQ ID NO: 93 - DNA, 48 bp, Artificial Sequence
  Primer
cataaatcataagaaattcgctcattttttcaacgcttccttttgagc

SEQ ID NO: 94 - DNA, 51 bp, Artificial Sequence
  Primer
cgaataaacacacataaacaaacaaaatgtctatagcaagttatgcccaag

SEQ ID NO: 95 - DNA, 47 bp, Artificial Sequence
  Primer
caggtggtcatggccctttttttgattaaaattaaaaaaactttttg

SEQ ID NO: 96 - DNA, 29 bp, Artificial Sequence
  Primer
tttttgattaaaattaaaaaaactttttg

SEQ ID NO: 97 - DNA, 58 bp, Artificial Sequence
  Primer
caaaaagtttttttaattttaatcaaaaaatgtcaatgagtaatattgttgtttttgg

SEQ ID NO: 98 - DNA, 51 bp, Artificial Sequence
  Primer
cgtcctctcgaaaggtggttaattcaaattaattgatatagttttttaatg

SEQ ID NO: 99 - DNA, 81 bp, Artificial Sequence
  Primer
aagggccatgaccacctgatgcaccaattaggtaggtctggctatgtctatacctctggcaattcgccctatagtgagtcg

SEQ ID NO: 100 - DNA, 87 bp, Artificial Sequence
  Primer
cacctttcgagaggacgatgcccgtgtctaaatgattcgaccagcctaagaatgttcaacgagctccagcttttgttccctttagtg

SEQ ID NO: 101 - DNA, 47 bp, Artificial Sequence
  Primer
gcatcgtcctctcgaaaggtgtcgagtttatcattatcaatactgcc

SEQ ID NO: 102 - DNA, 32 bp, Artificial Sequence; identical to SEQ ID NO: 90
  Primer

SEQ ID NO: 103 - DNA, 21 bp, Artificial Sequence
  Primer
gcgaatttcttatgatttatg

SEQ ID NO: 104 - DNA, 37 bp, Artificial Sequence
  Primer
gattaatataattatataaaaatattatcttcttttc

SEQ ID NO: 105 - DNA, 23 bp, Artificial Sequence
  Primer
ctggtttgttttacaaccaaaag

SEQ ID NO: 106 - DNA, 47 bp, Artificial Sequence
  Primer
aactctttcataaaatggtatctttaactttttatttaatcgtaatg

SEQ ID NO: 107 - DNA, 19 bp, Artificial Sequence
  Primer
aatgacaagtttcatcatc

SEQ ID NO: 108 - DNA, 54 bp, Artificial Sequence
  Primer
gcaatctaatctaagttttaattacaaagttactccgcaacgcttttctgaacg

SEQ ID NO: 109 - DNA, 56 bp, Artificial Sequence
  Primer
gcaatctaatctaagttttaattacaaaatgaacaggattaagaatacattttctg

SEQ ID NO: 110 - DNA, 47 bp, Artificial Sequence
  Primer
ctagtgtctcccgtcttctgtttaccaatagtttggaaataagtatc

SEQ ID NO: 111 - DNA, 55 bp, Artificial Sequence
  Primer
ctataactacaaaaaacacatacataaactaaaaatgaccttggcacccctagac

SEQ ID NO: 112 - DNA, 49 bp, Artificial Sequence
  Primer
gactaataattcttagttaaaagcacttcagttcaaatcagtaacaacc

SEQ ID NO: 113 - DNA, 50 bp, Artificial Sequence
  Primer
gtcgacgcgtaagcttgtgggccctatcatgatgctgtaatagcagaatc

SEQ ID NO: 114 - DNA, 51 bp, Artificial Sequence
  Primer
ctactttttacaacaaatataacaaaatggaaggtggtggtgctagaaatg

SEQ ID NO: 115 - DNA, 55 bp, Artificial Sequence
  Primer
ctataactacaaaaaacacatacataaactaaaaatgccagagtatacgctactg

SEQ ID NO: 116 - DNA, 52 bp, Artificial Sequence
  Primer
gactaataattcttagttaaaagcacttcatatatcatatctacgatcatcg

SEQ ID NO: 117 - DNA, 22 bp, Artificial Sequence
  Primer
gtgaaggaacaactcgtgtctc

SEQ ID NO: 118 - DNA, 22 bp, Artificial Sequence
  Primer
gaggatacgtacatatgcaagc

SEQ ID NO: 119 - DNA, 25 bp, Artificial Sequence
  Primer
tgccgtaaaccactaaatcggaacc

SEQ ID NO: 120 - DNA, 70 bp, Artificial Sequence
  Primer
tcagacttcttaactcctgtaaaaacaaaaaaaaaaaaaggcatagcaataagctggagctcatagcttc

SEQ ID NO: 121 - DNA, 81 bp, Artificial Sequence
  Primer
gtgcctattgatgatctggcggaatgtctgccgtgccatagccatgccttcacatatagtccgcaaattaaagccttcgag

SEQ ID NO: 122 - DNA, 81 bp, Artificial Sequence
  Primer
actatatgtgaaggcatggctatggcacggcagacattccgccagatcatcaataggcaccttcgtacgctgcaggtcgac

SEQ ID NO: 123 - DNA, 120 bp, Artificial Sequence
  Primer
tgcgcatgtttcggcgttcgaaacttctccgcagtgaaagataaatgatcactcatcaaattccgtacatgttttagagctagaaatagcaagttaaaataaggctagtccgttatcaac

SEQ ID NO: 124 - DNA, 120 bp, Artificial Sequence
  Primer
gttgataacggactagccttattttaacttgctatttctagctctaaaacatgtacggaatttgatgagtgatcatttatctttcactgcggagaagtttcgaacgccgaaacatgcgca

SEQ ID NO: 125 - DNA, 120 bp, Artificial Sequence
  Primer
ataaaatatcaaaacgccgatgagacaggcaggataaagtgacagattcagttatacatttttattagcattgatattattattttaaaaagtctatttacttgtatatttatccgaata

SEQ ID NO: 126 - DNA, 120 bp, Artificial Sequence
  Primer
tattcggataaatatacaagtaaatagactttttaaaataataatatcaatgctaataaaaatgtataactgaatctgtcactttatcctgcctgtctcatcggcgttttgatattttat

SEQ ID NO: 127 - DNA, 20 bp, Artificial Sequence
  Primer
ttttgcaacatccgggcatg

SEQ ID NO: 128 - DNA, 20 bp, Artificial Sequence
  Primer
cggctagctggtatggatcg

SEQ ID NO: 129 - DNA, 24 bp, Artificial Sequence
  Primer
atagcttcaaaatgtttctactcc

SEQ ID NO: 130 - DNA, 20 bp, Artificial Sequence
  Primer
tgcgacagaagaaagggaag

SEQ ID NO: 131 - DNA, 21 bp, Artificial Sequence
  Primer
cgtctatgaggagactgttag

SEQ ID NO: 132 - DNA, 43 bp, Artificial Sequence
  Primer
ggagtagaaacattttgaagctatacgtgaccacttcgagagc

SEQ ID NO: 133 - DNA, 52 bp, Artificial Sequence
  Primer
gcaatctaatctaagttttaattacaaaatgactgtcaccataaaagaattg

SEQ ID NO: 134 - DNA, 52 bp, Artificial Sequence
  Primer
gtgctagtgtctcccgtcttctgttcatattttcttctgcaatttcatatag

SEQ ID NO: 135 - DNA, 45 bp, Artificial Sequence
  Primer
gcttcggaaaatacgatgttgaaaatcctgcataatcggcctcac

SEQ ID NO: 136 - DNA, 20 bp, Artificial Sequence
  Primer
ctcgccaaggcattaccatc

SEQ ID NO: 137 - DNA, 19 bp, Artificial Sequence
  Primer
gagaacgagaggacccaac

SEQ ID NO: 138 - DNA, 44 bp, Artificial Sequence
  Primer
ggttccgatttagtggtttacggcaacgtgaccacttcgagagc

SEQ ID NO: 139 - DNA, 54 bp, Artificial Sequence
  Primer
gtcgacgcgtaagcttgtgggccctatcatattttcttctgcaatttcatatag

SEQ ID NO: 140 - DNA, 50 bp, Artificial Sequence
  Primer
ctactttttacaacaaatataacaaaatgactgtcaccataaaagaattg

SEQ ID NO: 141 - DNA, 52 bp, Artificial Sequence
  Primer
ggtagaccaatgtagcgctcttactttatcattttttcaacgcttccttttg

SEQ ID NO: 142 - DNA, 55 bp, Artificial Sequence
  Primer
gtttcgaataaacacacataaacaaacaaaatgtctatagcaagttatgcccaag

SEQ ID NO: 143 - DNA, 45 bp, Artificial Sequence
  Primer
ggcagtattgataatgataaactcgacctgcataatcggcctcac

SEQ ID NO: 144 - DNA, 49 bp, Artificial Sequence
  Primer
gactaataattcttagttaaaagcactctaccatccatgctcgaacaac

SEQ ID NO: 145 - DNA, 49 bp, Artificial Sequence; identical to SEQ ID NO: 144
  Primer

SEQ ID NO: 146 - DNA, 45 bp, Artificial Sequence
  Primer
gcatatgggagatggagatgatacctcctgcataatcggcctcac

SEQ ID NO: 147 - DNA, 36 bp, Artificial Sequence
  Primer
gggtaccggccgcaaattaaagccttcgagcgtccc

SEQ ID NO: 148 - DNA, 22 bp, Artificial Sequence
  Primer
gtgttcattgtacgtcctagac

SEQ ID NO: 149 - DNA, 19 bp, Artificial Sequence
  Primer
gtgcccaaagctaagagtc

SEQ ID NO: 150 - DNA, 18 bp, Artificial Sequence
  Primer
ctgctcttgaatggcgac

SEQ ID NO: 151 - DNA, 38 bp, Artificial Sequence
  Primer
gtcgccattcaagagcagcatcgtcctctcgaaaggtg

SEQ ID NO: 152 - DNA, 36 bp, Artificial Sequence
  Primer
cgaatcttcccatgcctgcaggtggtcatggccctt

SEQ ID NO: 153 - DNA, 18 bp, Artificial Sequence
  Primer
caggcatgggaagattcg

SEQ ID NO: 154 - DNA, 21 bp, Artificial Sequence
  Primer
ctggtgaggatttacggtatg

SEQ ID NO: 155 - DNA, 20 bp, Artificial Sequence
  Primer
gtgcgttatcgggttcttac

SEQ ID NO: 156 - DNA, 22 bp, Artificial Sequence
  Primer
caggttagttacttgctctatg

SEQ ID NO: 157 - DNA, 41 bp, Artificial Sequence
  Primer
cagtattgataatgataaactcgaaatcagacgcacgcttg

SEQ ID NO: 158 - DNA, 40 bp, Artificial Sequence
  Primer
ctttaatttgcggccggtacccttacgtggattgagccag

SEQ ID NO: 159 - DNA, 24 bp, Artificial Sequence
  Primer
gattgtcataataggagctatttg

SEQ ID NO: 160 - DNA, 25 bp, Artificial Sequence
  Primer
ccatagtattactattggtgttcat

SEQ ID NO: 161 - DNA, 22 bp, Artificial Sequence
  Primer
gttatcggttgtgatattgttc

SEQ ID NO: 162 - DNA, 22 bp, Artificial Sequence
  Primer
ttaagctattgtttcggcaatt

SEQ ID NO: 163 - DNA, 29 bp, Artificial Sequence
  Primer
cgtgcgauctctataaaaaatgtgcgaac

SEQ ID NO: 164 - DNA, 27 bp, Artificial Sequence
  Primer
atgacagautggtgttgtggttctgtg

SEQ ID NO: 165 - DNA, 21 bp, Artificial Sequence
  Primer
gagatctttgtgttcggttac

SEQ ID NO: 166 - DNA, 18 bp, Artificial Sequence
  Primer
agtctcgtatgtcggctc

SEQ ID NO: 167 - DNA, 18 bp, Artificial Sequence
  Primer
tgtgtccgcgtttctaag

SEQ ID NO: 168 - DNA, 21 bp, Artificial Sequence
  Primer
gaggtggttattgatcaccag

SEQ ID NO: 169 - DNA, 18 bp, Artificial Sequence
  Primer
acgaatcgttaggcacag

SEQ ID NO: 170 - DNA, 17 bp, Artificial Sequence
  Primer
gtgcaataccaaaatcg

SEQ ID NO: 171 - DNA, 28 bp, Artificial Sequence
  Primer
gcagttgtttggattaaaaagctgtacg

SEQ ID NO: 172 - DNA, 25 bp, Artificial Sequence
  Primer
ccttgtgtcatcatttactccaggc

SEQ ID NO: 173 - DNA, 26 bp, Artificial Sequence
  Primer
gtagagtcttagctgcagttggtatg

SEQ ID NO: 174 - DNA, 24 bp, Artificial Sequence
  Primer
cagggcattattactgacggcatg

SEQ ID NO: 175 - DNA, 26 bp, Artificial Sequence
  Primer
ctacagcacctttgaaagaaggtgtc

SEQ ID NO: 176 - DNA, 23 bp, Artificial Sequence
  Primer
gttgatggtgtcgtagacgtcag

SEQ ID NO: 177 - DNA, 20 bp, Artificial Sequence
  Primer
ggaacgtggatttaccccag

SEQ ID NO: 178 - DNA, 26 bp, Artificial Sequence
  Primer
gttatcggttgtgatattgttcctgc

SEQ ID NO: 179 - DNA, 20 bp, Artificial Sequence
  Primer
caaagcgatgggctccagac

SEQ ID NO: 180 - DNA, 23 bp, Artificial Sequence
  Primer
cattccgcagttaacatgtggtc

SEQ ID NO: 181 - DNA, 28 bp, Artificial Sequence
  Primer
gtgttcattgtacgtcctagactcaaac

SEQ ID NO: 182 - DNA, 21 bp, Artificial Sequence
  Primer
cgtgcgttatcgggttcttac

SEQ ID NO: 183 - DNA, 59 bp, Artificial Sequence
  Primer
ggaacactggggcaataggctgtcgccattcaagagcagcatcgtcctctcgaaaggtg

SEQ ID NO: 184 - DNA, 60 bp, Artificial Sequence
  Primer
ctattgtaattcaaaaaaaaaaagcgaatcttcccatgcctgcaggtggtcatggccctt

SEQ ID NO: 185 - DNA, 24 bp, Artificial Sequence
  Primer
cttgcataaattggtcaatgcaag

SEQ ID NO: 186 - DNA, 25 bp, Artificial Sequence
  Primer
cgatgacctcccattgatatttaag

SEQ ID NO: 187 - DNA, 24 bp, Artificial Sequence
  Primer
catcgtcaatttgtgatcgaagac

SEQ ID NO: 188 - DNA, 19 bp, Artificial Sequence
  Primer
cattcgccaggtagcttac

SEQ ID NO: 189 - DNA, 21 bp, Artificial Sequence
  Primer
tgcattttgagcgttgaacaa

SEQ ID NO: 190 - DNA, 20 bp, Artificial Sequence
  Primer
gtgccctgttctctgtagtt

SEQ ID NO: 191 - DNA, 60 bp, Artificial Sequence
  Primer
atcgggccctccttactgctctccttccgtgtaacgcgtttgccgtaaaccactaaatcg

SEQ ID NO: 192 - DNA, 57 bp, Artificial Sequence
  Primer
cgttaagaaaaatttcgagagagtcgccgatagtagattttcaacatcgtattttcc

SEQ ID NO: 193 - DNA, 57 bp, Artificial Sequence
  Primer
cctccttactgctctccttccgtgtaacgcgttatagcttcaaaatgtttctactcc

SEQ ID NO: 194 - DNA, 30 bp, Artificial Sequence
  Primer
ttatcgagctaactattttcgacacacatg

SEQ ID NO: 195 - DNA, 22 bp, Artificial Sequence
  Primer
cgtcgcccagtaagtgagacta

SEQ ID NO: 196 - DNA, 59 bp, Artificial Sequence
  Primer
gaaagcatagcaatctaatctaagttttaattacaaaatgtcaatgagtaatattgttg

SEQ ID NO: 197 - DNA, 51 bp, Artificial Sequence
  Primer
aagttgtgtgctagtgtctcccgtcttctgtctaccatccatgctcgaaca

SEQ ID NO: 198 - DNA, 60 bp, Artificial Sequence
  Primer
ctcgcctagtaaataaacgataaacaaatttgaagtagtagatacacgtatctcgacatg

SEQ ID NO: 199 - DNA, 59 bp, Artificial Sequence
  Primer
gaatgcaacaccgtagcatgaatcttgagattgcatctgataatgggttagtagtttat

SEQ ID NO: 200 - DNA, 70 bp, Artificial Sequence
  Primer
tctccgcagtgaaagataaatgatcaatttacgaaaaataaaggcgttttagagctagaaatagcaagtt

SEQ ID NO: 201 - DNA, 70 bp, Artificial Sequence
  Primer
aacttgctatttctagctctaaaacgcctttatttttcgtaaattgatcatttatctttcactgcggaga

SEQ ID NO: 202 - DNA, 60 bp, Artificial Sequence
  Primer
aataaaggcaaaaacagtggtcgtgtgagaaatctattttttcgaaattacttacacttt

SEQ ID NO: 203 - DNA, 60 bp, Artificial Sequence
  Primer
aaagtgtaagtaatttcgaaaaaatagatttctcacacgaccactgtttttgcctttatt

SEQ ID NO: 204 - DNA, 20 bp, Artificial Sequence
  Primer
ggtcacccacccatatacgg

SEQ ID NO: 205 - DNA, 20 bp, Artificial Sequence
  Primer
tgtcctccggataactgcac

SEQ ID NO: 206 - DNA, 120 bp, Artificial Sequence
  Primer
tgcgcatgtttcggcgttcgaaacttctccgcagtgaaagataaatgatcaatttacgaaaaataaaggcgttttagagctagaaatagcaagttaaaataaggctagtccgttatcaac

SEQ ID NO: 207 - DNA, 120 bp, Artificial Sequence
  Primer
gttgataacggactagccttattttaacttgctatttctagctctaaaacgcctttatttttcgtaaattgatcatttatctttcactgcggagaagtttcgaacgccgaaacatgcgca

SEQ ID NO: 208 - DNA, 120 bp, Artificial Sequence
  Primer
tctttttttgttcccaacaagaagtgagttaataaaggcaaaaacagtggtcgtgtgagaaatctattttttcgaaattacttacacttttgacggctagaaaaggatatacatacatat

SEQ ID NO: 209 - DNA, 120 bp, Artificial Sequence
  Primer
atatgtatgtatatccttttctagccgtcaaaagtgtaagtaatttcgaaaaaatagatttctcacacgaccactgtttttgcctttattaactcacttcttgttgggaacaaaaaaaga

SEQ ID NO: 210 - DNA, 120 bp, Artificial Sequence
  Primer
tgcgcatgtttcggcgttcgaaacttctccgcagtgaaagataaatgatcatcttcaaatccactacatagttttagagctagaaatagcaagttaaaataaggctagtccgttatcaac

SEQ ID NO: 211 - DNA, 120 bp, Artificial Sequence
  Primer
gttgataacggactagccttattttaacttgctatttctagctctaaaactatgtagtggatttgaagatgatcatttatctttcactgcggagaagtttcgaacgccgaaacatgcgca

SEQ ID NO: 212 - DNA, 120 bp, Artificial Sequence
  Primer
ttgtacgcttcacatagtagttcagtcaagaagagcaaacactaataagcaataaatctaggagaatatacatatatatgcatatgtttgtttagctaaataattttattgagctttgct

SEQ ID NO: 213 - DNA, 120 bp, Artificial Sequence
  Primer
agcaaagctcaataaaattatttagctaaacaaacatatgcatatatatgtatattctcctagatttattgcttattagtgtttgctcttcttgactgaactactatgtgaagcgtacaa

SEQ ID NO: 214 - DNA, 21 bp, Artificial Sequence
  Primer
acaccaatattctgcacctgc

SEQ ID NO: 215 - DNA, 20 bp, Artificial Sequence
  Primer
tgctggagaagatcgtacgc

SEQ ID NO: 216 - DNA, 120 bp, Artificial Sequence
  Primer
tgcgcatgtttcggcgttcgaaacttctccgcagtgaaagataaatgatcttagtagtttttggaaggatgttttagagctagaaatagcaagttaaaataaggctagtccgttatcaac

SEQ ID NO: 217 - DNA, 120 bp, Artificial Sequence
  Primer
gttgataacggactagccttattttaacttgctatttctagctctaaaacatccttccaaaaactactaagatcatttatctttcactgcggagaagtttcgaacgccgaaacatgcgca

SEQ ID NO: 218 - DNA, 120 bp, Artificial Sequence
  Primer
ttcttttttatattttttaggttttcatatagtgtcttacgcaaataggcggaccatagaaaagccgccatttgtgtctcctcatacttacatagaatagccctcttctattatccttcg

SEQ ID NO: 219 - DNA, 120 bp, Artificial Sequence
  Primer
cgaaggataatagaagagggctattctatgtaagtatgaggagacacaaatggcggcttttctatggtccgcctatttgcgtaagacactatatgaaaacctaaaaaatataaaaaagaa

SEQ ID NO: 220 - DNA, 20 bp, Artificial Sequence
  Primer
tacagctcgctccttgcatc

SEQ ID NO: 221 - DNA, 20 bp, Artificial Sequence
  Primer
gcttgcttggagggcttttc

SEQ ID NO: 222 - DNA, 120 bp, Artificial Sequence
  Primer
tgcgcatgtttcggcgttcgaaacttctccgcagtgaaagataaatgatcaagaaccctttatcataattgttttagagctagaaatagcaagttaaaataaggctagtccgttatcaac

SEQ ID NO: 223 - DNA, 120 bp, Artificial Sequence
  Primer
gttgataacggactagccttattttaacttgctatttctagctctaaaacaattatgataaagggttcttgatcatttatctttcactgcggagaagtttcgaacgccgaaacatgcgca

SEQ ID NO: 224 - DNA, 120 bp, Artificial Sequence
  Primer
ttttttgattgttctacaactttttcatagtaatcaaaacctttgaatttcaaacttactaggatatatttaaccacgactttcgcaagagagacggagggggtgggaaaaggctgaatg

SEQ ID NO: 225 - DNA, 120 bp, Artificial Sequence
  Primer
cattcagccttttcccaccccctccgtctctcttgcgaaagtcgtggttaaatatatcctagtaagtttgaaattcaaaggttttgattactatgaaaaagttgtagaacaatcaaaaaa

SEQ ID NO: 226 - DNA, 20 bp, Artificial Sequence
  Primer
tcatccaggtttcagcacgg

SEQ ID NO: 227 - DNA, 20 bp, Artificial Sequence
  Primer
agctcgaacaaggtgtcagg

SEQ ID NO: 228 - DNA, 60 bp, Artificial Sequence
  Primer
gattacttaccaatgtgccataaactccgtgcaccaatagcttcaaaatgtttctactcc

SEQ ID NO: 229 - DNA, 60 bp, Artificial Sequence
  Primer
cttgggttgtgggcaattgggtgtactatgaagcattttcaacatcgtattttccgaagc

SEQ ID NO: 230 - DNA, 47 bp, Artificial Sequence
  Primer
gcaatctaatctaagttttaattacaaaatggcacaagaaatcactc

SEQ ID NO: 231 - DNA, 45 bp, Artificial Sequence
  Primer
gtgctagtgtctcccgtcttctgtctaatttaattccttggctgc

SEQ ID NO: 232 - DNA, 25 bp, Artificial Sequence
  Primer
gaccatcactaaagcttctctctta

SEQ ID NO: 233 - DNA, 25 bp, Artificial Sequence
  Primer
ttgagcaattcatcgacaacaagag

SEQ ID NO: 234 - DNA, 43 bp, Artificial Sequence
  Primer
agaaccagaaccagatcctattttcttctgcaatttcatatag

SEQ ID NO: 235 - DNA, 37 bp, Artificial Sequence
  Primer
ggatctggttctggttctatggcacaagaaatcactc

SEQ ID NO: 236 - DNA, 36 bp, Artificial Sequence
  Primer
agaaccagaaccagatccatttaattccttggctgc

SEQ ID NO: 237 - DNA, 42 bp, Artificial Sequence
  Primer
ggatctggttctggttctatgactgtcaccataaaagaattg

SEQ ID NO: 238 - DNA, 60 bp, Artificial Sequence
  Primer
gattacttaccaatgtgccataaactccgtgcaccatgccgtaaaccactaaatcggaac

SEQ ID NO: 239 - DNA, 120 bp, Artificial Sequence
  Primer
tgcgcatgtttcggcgttcgaaacttctccgcagtgaaagataaatgatcttcttagattactattatatgttttagagctagaaatagcaagttaaaataaggctagtccgttatcaac

SEQ ID NO: 240 - DNA, 120 bp, Artificial Sequence
  Primer
gttgataacggactagccttattttaacttgctatttctagctctaaaacatataatagtaatctaagaagatcatttatctttcactgcggagaagtttcgaacgccgaaacatgcgca

SEQ ID NO: 241 - DNA, 120 bp, Artificial Sequence
  Primer
atagttattttgaaataataactaccattagaactaacaaaagaaaagaaaaaaaaaatataccatttgcaagacattgtataatatttttgttgaaagtctttttcgattcataagcgc

SEQ ID NO: 242 - DNA, 120 bp, Artificial Sequence
  Primer
gcgcttatgaatcgaaaaagactttcaacaaaaatattatacaatgtcttgcaaatggtatattttttttttcttttcttttgttagttctaatggtagttattatttcaaaataactat

SEQ ID NO: 243 - DNA, 20 bp, Artificial Sequence
  Primer
ctcatcgcatgccaacgaag

SEQ ID NO: 244 - DNA, 20 bp, Artificial Sequence
  Primer
gcagcaaagccaacccttac

SEQ ID NO: 245 - DNA, 49 bp, Artificial Sequence
  Primer
gcaatctaatctaagttttaattacaaaatgtctgttcactctatcttg

SEQ ID NO: 246 - DNA, 45 bp, Artificial Sequence
  Primer
gtgctagtgtctcccgtcttctgtttattcaaccatcttctttgg

SEQ ID NO: 247 - DNA, 45 bp, Artificial Sequence
  Primer
caatgtagcgctcttactttattatttcaagtccttgaaattacc

SEQ ID NO: 248 - DNA, 22 bp, Artificial Sequence
  Primer
ctggagctcagtttatcattat

SEQ ID NO: 249 - DNA, 21 bp, Artificial Sequence
  Primer
actatagggcgaattgggtac

SEQ ID NO: 250 - DNA, 40 bp, Artificial Sequence
  Primer
ataatgataaactgagctccagagtctcgtatgtcggctc

SEQ ID NO: 251 - DNA, 39 bp, Artificial Sequence
  Primer
gtacccaattcgccctatagttgtgtccgcgtttctaag

SEQ ID NO: 252 - DNA, 52 bp, Artificial Sequence
  Primer
ggtagaccaatgtagcgctcttactttattattcaaccatcttctttggaac

SEQ ID NO: 253 - DNA, 52 bp, Artificial Sequence
  Primer
gtttcgaataaacacacataaacaaacaaaatgtctgttcactctatcttgt

SEQ ID NO: 254 - DNA, 57 bp, Artificial Sequence
  Primer
ctataactacaaaaaacacatacataaactaaaaatgccgtttggaatagacaacac

SEQ ID NO: 255 - DNA, 50 bp, Artificial Sequence
  Primer
gactaataattcttagttaaaagcactttaccagacatcttcttggtatc

SEQ ID NO: 256 - DNA, 19 bp, Artificial Sequence
  Primer
tggtcacacaacttgtctg

SEQ ID NO: 257 - DNA, 20 bp, Artificial Sequence
  Primer
ggtactggtggtttcacttg

SEQ ID NO: 258 - DNA, 21 bp, Artificial Sequence
  Primer
gtagtgatcattggcttaacg

SEQ ID NO: 259 - DNA, 46 bp, Artificial Sequence
  Primer
gttccgatttagtggtttacggcagtgacaataaattcaaaccggt

SEQ ID NO: 260 - DNA, 46 bp, Artificial Sequence
  Primer
gcttcggaaaatacgatgttgaaaatcaactcagaagtttgacagc

SEQ ID NO: 261 - DNA, 21 bp, Artificial Sequence
  Primer
tcgttagattctgtatcccta

SEQ ID NO: 262 - DNA, 46 bp, Artificial Sequence
  Primer
ggagtagaaacattttgaagctatgtgacaataaattcaaaccggt

SEQ ID NO: 263 - DNA, 22 bp, Artificial Sequence
  Primer
atttgtgatcgaagacgaagag

SEQ ID NO: 264 - DNA, 18 bp, Artificial Sequence
  Primer
tcaagaagccactacgtg

SEQ ID NO: 265 - DNA, 24 bp, Artificial Sequence
  Primer
actagaacattaccatatgtagtg

SEQ ID NO: 266 - DNA, 20 bp, Artificial Sequence
  Primer
caacttggacgttcttctac